(19) United States
(12) Patent Application Publication
(10) Pub. No.: US 2025/0263496 A1
(43) Pub. Date: Aug. 21, 2025

(54) Neutrophil-Activating Therapy for the Treatment of Cancer

(71) Applicant: The Board of Trustees of the Leland Stanford Junior University, Stanford, CA (US)

(72) Inventors: Edgar G. Engleman, Atherton, CA (US); Ian Linde, Redwood City, CA (US); Tyler Prestwood, Redwood City, CA (US); Markus Isfeld Diehl, Redwood City, CA (US)

(21) Appl. No.: 19/033,347

(22) Filed: Jan. 21, 2025

Related U.S. Application Data
(60) Provisional application No. 63/625,448, filed on Jan. 26, 2024.

(51) Int. Cl. C07K 16/28; A61K 38/19; A61K 39/00; A61K 45/06; A61P 35/00; A61P 35/04

(52) U.S. Cl. CPC C07K 16/2878; A61K 38/191; A61K 45/06; A61P 35/00; A61P 35/04; A61K 2039/507

(57) ABSTRACT

Provided herein are methods of treating cancer by novel treatments that induce neutrophil recruitment and activation. The treatments encompass a combination of TNF, a CD40 signaling agonist, and an antibody against a tumor antigen. Treatment with this combination surprisingly result in the infiltration and activation of tumor-killing neutrophils that drive T cell-independent tumor clearance. By this treatment, the tumor microenvironment is modulated to promote neutrophil-mediated inflammation and a tumor-eradicating immune response.

### CROSS-REFERENCE TO RELATED APPLICATIONS

This application claims the benefit of U.S. Provisional Patent Application No. 63/625,448, filed Jan. 26, 2024, which application is incorporated herein by reference in its entirety.

### STATEMENT REGARDING FEDERALLY SPONSORED RESEARCH

This invention was made with Government support under contracts CA196029, CA222969, and CA244114 awarded by the National Institutes of Health. The Government has certain rights in the invention.

### BACKGROUND

Although initially appreciated for their role in defense against microbial pathogens, neutrophils are now recognized to promote the growth and spread of many cancers.¹⁻³ Cancers are frequently accompanied by neutrophil recruitment to the tumor and expansion in the blood, which is associated with poor prognosis in most cases.^{1,2,4,5} Studies of neutrophils from cancer patients and mouse models have established that neutrophils promote tumor growth,⁶ angiogenesis,^{7,8} and metastasis⁹⁻¹¹ and inhibit anti-cancer T cell responses.^{9,12,13} Furthermore, myeloid-derived suppressor cells (MDSCs), a heterogeneous group of cells that overlap phenotypically with neutrophils,^{1,2,14} are well appreciated to induce T cell suppression and promote tumor growth and metastasis.^{2,14-16} Nonetheless, neutrophils have the potential to exert anti-tumor activity. Early studies demonstrated the ability of neutrophils to kill tumor cells in vitro,¹⁷ and although neutrophils exert pro-tumor activity in most settings,^{1,2} a growing number of studies support the potential for neutrophils to perform anti-tumor functions in certain contexts. Neutrophils naturally can inhibit some tumors during the early stages of tumor development¹⁸⁻²¹ or early metastasis,^{22,23} and they are capable of promoting anti-tumor responses by other immune cells, including natural killer (NK) cells and multiple subsets of T cells.^{19-21,24-26} The apparently contradictory roles of neutrophils in cancer are likely the result of differences in the tumor milieu affecting neutrophil maturation, activation, and functional states.^{2,24,27} Despite the natural capacity of neutrophils to inhibit cancer in certain contexts, little effort has been made to harness neutrophils as anti-tumor effector cells, and it is still not clear whether neutrophils can be harnessed therapeutically to drive regression of established tumors.

### SUMMARY

Provided are combination therapies which promote the recruitment of cancer-killing neutrophils to tumors in great numbers. These treatment methods and compositions, unexpectedly, modulate the tumor microenvironment to optimally recruit neutrophils and activate their cytotoxic function.

The provided methods and compositions include the use of three agents, TNF, a CD40 agonist, and a tumor-binding antibody. Administration of the three agents to a subject having cancer results in the modulation of the tumor microenvironment to facilitate neutrophil activation and clearance of tumors, as well as other therapeutic effects described herein.

It is noted that previous cancer research has contemplated a combination treatment comprising TNF, a CD40 receptor agonist, and an anti-tumor allogeneic Ig, for example as described in Carmi et al., 2015. Allogeneic IgG combined with dendritic cell stimuli induces anti-tumor T cell immunity, Nature. 2015 May 7; 521(7550): 99-104. doi:10.1038/nature14424.

That work describes the administration of lower doses of TNF than contemplated in the present disclosure, and while certain tumor immunity therapeutic effects were observed, no neutrophil recruitment, activation, or cell-killing effects were observed. In contrast, the methods and compositions of the present disclosure utilize higher doses of TNF or TNF agent and result in the previously unobserved infiltration of tumors with activated neutrophils.

In some cases, the route of administration is local, e.g., intratumoral. However, in some cases, the route of administration will be systemic, e.g., subcutaneous (e.g., in some cases for the purpose of preventing metastasis). In some such cases, the primary tumor is inaccessible or has already been removed by surgery or radiation, and yet administration, e.g., systemic administration such as subcutaneous, can still function to prevent metastasis. For example, Example 2 of the experimental section below (“Examples”), demonstrate that, surprisingly, Neutrophil-activating therapy (NAT) when administered subcutaneously, still works for purposes of preventing metastasis. In addition, surprisingly, the results of Example 2 show that NAT is effective even if the induction of metastasis is delayed for two weeks following NAT. These results show that direct injection into a tumor is not required (i.e., systemic administration such as subcutaneous administration is effective) for the metastasis benefit, indicating that the treatment can be administered in a manner that protects patients from metastasis at sites distant from the site of NAT administration, and even in situations where the primary tumor is inaccessible or has already been removed by surgery or radiation.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1K: Neutrophil-activating therapy recruits activated neutrophils to the tumor. (FIG. 1A) Left: tumor growth in B16-bearing mice following treatment with the indicated components, indicating mice with undetectable tumors at the conclusion of the study in parentheses. Right: survival of the mice shown in the left panel. Mice were euthanized when tumors exceeded 100 mm². (FIG. 1B and FIG. 1C) Neutrophil frequency (FIG. 1B) and numbers (FIG. 1C) in B16 tumors following treatment with TNF+anti-CD40+anti-gp75 (n=5). (FIG. 1D) Neutrophil frequency in peripheral blood following treatment with this neutrophil-activating therapy (n=5). (FIG. 1E) Immunofluorescence of neutrophil infiltration in B16 tumors following treatment with neutrophil-activating therapy. Scale bars, 500 mm. (FIG. 1F-1H) Neutrophil frequency in the tumor (F) and blood (G and H) 4 h (G) or 24 h (F and H) after treatment with the indicated components (n=4). (FIG. 1I) Frequencies of HSCs and progenitors in the bone marrow 24 h after treatment with neutrophil-activating therapy (anti-gp75, n=4; other groups, n=5). (FIG. 1J) Representative histograms (top) and median fluorescence intensity (MFI) (bottom) of surface markers on neutrophils infiltrating B16 tumors 4 h after treatment with the indicated components (n=4). (FIG. 1K) Surface marker expression on B16 tumor-infiltrating neutrophils following treatment with the full neutrophil-activating therapy (n=4). Statistics: log rank test with Bonferroni correction (A), one-way ANOVA with Tukey's multiple-comparisons test (B-D and F-K). For all dot plots, the line indicates the mean. Data are representative of 2 (B-E) or 3 (F-H) independent experiments or pooled from 2 experiments (A).

FIG. 2A-2Q: Therapeutically activated neutrophils eradicate multiple tumor types and reduce metastatic seeding. (FIG. 2A) Lysis of B16 cells co-cultured with neutrophils isolated from treated tumors and stimulated in vitro with the indicated components (n=4). (FIG. 2B) Lysis of B16 cells co-cultured with neutrophils isolated from treated tumors or tumor-naive bone marrow (BM), stimulated in vitro with TNF+anti-CD40+anti-gp75 (n=4). (FIG. 2C) Lysis of B16 cells co-cultured with neutrophils isolated from treated tumors in WT or Fcerig mice, stimulated in vitro with TNF+anti-CD40+anti-gp75 or isotype control, with or without anti-CD16/CD32 to block FcgRs (n=4). (FIG. 2D) Signal from anti-gp75-Alexa Fluor 647 in neutrophils isolated from treated tumors or naive bone marrow and cultured in vitro with B16 cells together with TNF+anti-CD40+anti-gp75-Alexa Fluor 647 or no stimulation (BM, n=3; tumor, n=4). (FIG. 2E and FIG. 2F) Percentage DiD⁺ neutrophils (E) and DiD MFI in DiD⁺ neutrophils (FIG. 2F) following co-culture of treated tumor neutrophils with DiD-labeled B16 and stimulation in vitro with the indicated components (unstained/triple, n=4; unstimulated/double, n=3). (FIG. 2G) Survival of B16-bearing WT or Fcerlg mice following treatment with neutrophil-activating therapy (n=10). (FIG. 2H) Regimen for neutrophil depletion and therapy. Treatment was performed 4 h after administration of anti-Ly6G or isotype control on days 0 and 2. (FIG. 2I and FIG. 2J) Representative TUNEL immunofluorescence (I) and quantification (J) in B16 tumors 24 h after treatment with neutrophil-activating therapy, following neutrophil depletion with anti-Ly6G or isotype control. Scale bars, 500 mm. (isotype, n=3; others, n=4). (FIG. 2K) Survival of B16-bearing mice administered anti-Ly6G or isotype control prior to neutrophil-activating therapy (n=10). (FIG. 2L-2N) Survival of mice bearing LL/2 (L) (mock, n=8; others, n=10), 4T1 (M) (n=10), and Sparkl.4640 (N) (mock, n=8; isotype, n=10; anti-Ly6G, n=9) tumors administered anti-Ly6G or isotype control prior to neutrophil-activating therapy. (FIG. 20) Percentage of MMTV-PyMT mice with treated tumors below the threshold of 100 mm² following treatment of one tumor per mouse in the context of anti-Ly6G or isotype control (mock, n=8; others, n=6). (FIG. 2P) Representative images of B16-tdTomato fluorescence in the lung (left) and quantification of the number and average area of tdTomato⁺ lung metastases (right) in mice bearing subcutaneous (s.c.) B16 tumors that were injected intravenously through the tail vein with B16-tdTomato one week after tumor implantation. Ten hours after tail vein injection, s.c. tumors were treated with mock or neutrophil-activating therapy, and the lungs were harvested and imaged 9 days after the tail vein injection. Lung borders are outlined in white. Scale bars, 1 mm (mock, n=8; treated, n=9). (FIG. 2Q) Representative image of India ink-stained lungs (left) and number of lung metastases (right) 30 days after orthotopic implantation of 4T1, in mice receiving neutrophil-activating therapy or mock treatment (mock, n=8; treated, n=9). Statistics: two-way ANOVA with Tukey's multiple-comparisons test (A-D), one-way ANOVA with Tukey's multiple-comparisons test (E, F, and J), log rank test (F and K), log rank test with Bonferroni correction (L-O), unpaired two-tailed t test (P and Q). For all dot plots, the line indicates the mean. Data are representative of 2 (A-J and P) or 3 (K) independent experiments or pooled from 2 (Q), 3 (L-N), or 6 (0) experiments.

FIG. 3A-3S: Therapy activates antigen-presenting cells and primes T cell memory. (FIG. 3A) Frequency of immune cell subsets in B16 tumors 24 h after treatment (n=4). (FIG. 3B) Percentage of cDC2s out of total DCs in B16 tumors 24 h after treatment (n=4). (FIG. 3C) Percentage of T cell subsets out of total T cells in B16 tumors 24 h after treatment (n=4). (FIG. 3D) Representative histograms and MFIs for markers expressed on APC populations in B16 tumors 24 h after treatment (n=4). (FIG. 3E and FIG. 3F) Frequencies (E) and numbers (F) of T cells in the blood 7 days after treatment of B16 (anti-gp75, n=4; others, n=5). (FIG. 3G) Percentage of T cell subsets out of total T cells in the blood 7 days after treatment of B16 (anti-gp75, n=4; others, n=5). (FIG. 3H) Memory and effector phenotypes of T cell subsets in the blood 7 days after treatment of B16 (anti-gp75, n=4; others, n=5). (FIG. 3I) Representative histograms and MFIs for markers expressed on T cell subsets in the blood 7 days after treatment (anti-gp75, n=4; others, n=5). (FIG. 3J and FIG. 3K) Frequencies (FIG. 3J) and numbers (FIG. 3K) of T cells in the dLN 7 days after treatment of B16 (anti-gp75, n=4; others, n=5). (FIG. 3L) Percentage of T cell subsets out of total T cells in the dLN 7 days after treatment of B16 (anti-gp75, n=4; others, n=5). (FIG. 3M) Memory and effector phenotypes of T cell subsets in the dLN 7 days after treatment of B16 (anti-gp75, n=4; others, n=5). (N) Representative histograms and MFIs for markers expressed on T cell subsets in the dLN 7 days after treatment (anti-gp75, n=4; others, n=5). (FIG. 3O and FIG. 3P) Frequencies (FIG. 3O) and numbers (FIG. 3P) of T cells in the tumor 7 days after treatment of B16 (anti-gp75, n=4; others, n=5). (FIG. 3Q) Percentage of T cell subsets out of total T cells in the tumor 7 days after treatment of B16 (anti-gp75, n=4; others, n=5). (FIG. 3R) Survival of B16-bearing WT or Rag2^{/} mice treated with neutrophil-activating therapy (n=15). (FIG. 3S) Survival of WT or Rag2^{/} mice following implantation of B16 in tumor-naive or B16-cleared mice 50 days after initial treatment with neutrophil-activating therapy (WT cleared, n=14; Rag2 cleared, n=18; WT naive, n=10; Rag2^{/} naive, n=15). Statistics: two-way ANOVA with Tukey's multiple-comparisons test (FIG. 3A, FIGS. 3E-3I, FIGS. 3L-3N, and FIG. 3Q), one-way ANOVA with Tukey's multiple-comparisons test (FIG. 3B-3D, FIG. 3J, FIG. 3K, FIG. 3O, and FIG. 3P), log rank test (R), log rank test with Bonferroni correction (FIG. 3S). For all dot plots, the line indicates the mean. Data are representative of 2 (FIGS. 3A-3Q and FIG. 3S) or 3 (FIG. 3R) independent experiments.

FIGS. 4A-4L: Complement activates tumor-infiltrating neutrophils through C5AR1. (FIG. 4A) Deposition of C3 on B16 tumor-infiltrating neutrophils following treatment with neutrophil-activating therapy (n=5). (FIG. 4B and FIG. 4C) Representative immunofluorescence (FIG. 4B) and quantification (FIG. 4C) of C3 staining in B16 tumors after treatment. Scale bars, 500 mm (0 h, n=3; others, n=4). (FIG. 4D and FIG. 4E) Representative immunofluorescence (FIG. 4D) and quantification (FIG. 4E) of TUNEL staining in B16 tumors 24 h after treatment of mice that had received CVF or vehicle prior to treatment. Scale bars, 500 mm (mock, n=4; vehicle, n=5; CVF, n=6). (FIG. 4F) Survival of B16-bearing mice administered CVF prior to treatment with neutrophil-activating therapy (n=5). (FIG. 4G-4I) Survival of B16-bearing mice administered anti-Factor B (FIG. 4G) (n=7), anti-C5 (FIG. 4H) (isotype, n=12; anti-C5, n=5), and anti-C5AR1 (I) (n=5) blocking antibodies prior to treatment. (FIG. 4J) Expression of CD11b on B16 tumor-infiltrating neutrophils 4 h after treatment following CVF or vehicle administration (vehicle, n=4; others, n=5). (FIG. 4K) Expression of CD11b on naive neutrophils following stimulation in vitro with the indicated factors (n=8). (FIG. 4L) Lysis of B16 cells co-cultured with neutrophils isolated from treated tumors and stimulated in vitro with the indicated factors (n=4). Statistics: two-way ANOVA with Tukey's multiple-comparisons test (FIG. 4A and FIG. 4L), one-way ANOVA with Tukey's multiple-comparisons test (FIG. 4C, FIG. 4E, FIG. 4J, and FIG. 4K), log rank test (FIG. 4F-4I). For all dot plots, the line indicates the mean. Data are representative of 2 (FIG. 4A-4C, FIG. 4F, FIG. 4G, and FIG. 4I-4L) or 3 (FIG. 4D and FIG. 4E) independent experiments or pooled from 2 experiments (FIG. 4H).

FIG. 5A-5H: Secretion of leukotriene B4 by C5a-activated neutrophils drives tumor clearance. (FIG. 5A) LTB₄ levels in B16 tumors 24 h after treatment with neutrophil-activating therapy following neutrophil depletion by anti-Ly6G (mock, n=7; others, n=6). (FIG. 5B) LTB₄ produced ex vivo by cells harvested from B16 tumors 12 h after treatment (n=11). (FIG. 5C) LTB₄ levels in B16 tumors 24 h after treatment following administration of CVF (n=6). (FIG. 5D) LTB₄ production by naive neutrophils following stimulation in vitro with the indicated factors (n=8). (FIG. 5E) Quantification of TUNEL staining in B16 tumors 24 h after treatment with neutrophil-activating therapy following administration of SC57461A (vehicle, n=3; others, n=4). (FIG. 5F and FIG. 5G) Survival of B16-bearing mice after treatment following administration of SC57461A (FIG. 5F) (vehicle, n=9; SC57461A, n=8) or CP-105696 (FIG. 5G) (vehicle, n=9; CP-105696, n=10). (FIG. 5H) Lysis of B16 cells co-cultured with neutrophils isolated from treated tumors and stimulated in vitro with neutrophil-activating therapy together with the indicated inhibitors (n=4).

Statistics: one-way ANOVA with Tukey's multiple-comparisons test (FIG. 5A and FIG. 5C-5E), repeated-measures one-way ANOVA with Tukey's multiple-comparisons test (FIG. 5B), log rank test (FIG. 5F and FIG. 5G), two way ANOVA with Tukey's multiple-comparisons test (FIG. 5H). For all dot plots, the line indicates the mean. Data are representative of 2 (FIG. 5A, FIG. 5C, FIG. 5D, and FIG. 5H) or 1 (FIG. 5E) independent experiment or pooled from 2 experiments (FIG. 5B, FIG. 5F, and FIG. 5G).

FIG. 6A-6N: LTB₄-dependent induction of xanthine oxidase induces oxidative damage and tumor clearance. (FIG. 6A and FIG. 6B) Representative immunofluorescence (FIG. 6A) and quantification (FIG. 6B) of DNA/RNA oxidative damage in B16 tumors 24 h post-treatment with neutrophil-activating therapy. Scale bars, 500 mm (n=4). (FIG. 6C-6E) Percentage of oxidized glutathione in B16 lysates 24 h after treatment with neutrophil-activating therapy following administration of anti-Ly6G (C) (mock, n=5; others, n=6), CVF (FIG. 6D) (mock, n=4; vehicle, n=7; CVF, n=6), or SC57461A (E) (mock, n=5; vehicle, n=8; SC57461A, n=7). (FIG. 6F) Lysis of B16 cells co-cultured with neutrophils isolated from treated tumors and stimulated in vitro with neutrophil-activating therapy together with the indicated inhibitors (n=4). (FIG. 6G) Survival of B16-bearing mice treated with neutrophil-activating therapy following administration of catalase (n=9). (FIG. 6H-6J) XO activity in the tumor 24 h after treatment of B16 with neutrophil-activating therapy following administration of anti-Ly6G (FIG. 6H) (isotype, n=7; others, n=8), CVF (I) (n=7), or SC57461A (J) (n=7). (FIG. 6K) Percentage of oxidized glutathione in B16 lysates 24 h after treatment following administration of topiroxostat (n=5). (FIG. 6L) Quantification of TUNEL staining in B16 tumors 24 h after treatment with neutrophil-activating therapy following administration of topiroxostat (n=3). (FIG. 6M) Survival of B16-bearing mice treated with neutrophil-activating therapy following administration of topiroxostat (vehicle, n=9; topiroxostat, n=8). (FIG. 6N) Lysis of B16 cells co-cultured with neutrophils isolated from treated tumors and stimulated in vitro with neutrophil-activating therapy in the presence of topiroxostat (n=4). Statistics: one-way ANOVA with Tukey's multiple-comparisons test (FIGS. 6B-6E and FIGS. 6H-6L), two-way ANOVA with Tukey's multiple-comparisons test (FIG. 6F and FIG. 6N), log rank test (FIG. 6G and FIG. 6M). For all dot plots, the line indicates the mean. Data are representative of 2 (FIG. 6F, FIGS. 6I-6K, FIG. 6M, and FIG. 6N) or 1 (FIG. 6A, FIG. 6B, and FIG. 6L) independent experiment or pooled from 2 experiments (FIGS. 6C-6E, FIG. 6G, and FIG. 6H).

FIG. 7A-7E Neutrophil-activating therapy activates human neutrophils to kill tumors. (FIG. 7A and FIG. 7B) Survival of Rag2^{/} Il2rg^{/} mice bearing subcutaneous A549 (FIG. 7A) (anti-Ly6G, n=7; others, n=6) or orthotopic MDA-MB-231 (FIG. 7B) (anti-Ly6G, n=7; others, n=6) treated with neutrophil-activating therapy following anti-Ly6G administration. (FIG. 7C) Cell surface markers on neutrophils from the peripheral blood of healthy human donors following stimulation with the indicated components for 30 min (ICAM-1, n=6; others, n=8). (FIG. 7D and FIG. 7E) Lysis of A549 cells co-cultured with neutrophils isolated from healthy donor peripheral blood and stimulated in vitro with the indicated components (n=4).

Statistics: log-rank test with Bonferroni correction (FIG. 7A and FIG. 7B), one-way ANOVA with Tukey's multiple-comparisons test (FIG. 7C), two-way ANOVA with Tukey's multiple-comparisons test (FIG. 7D and FIG. 7E). For all dot plots, the line indicates the mean. Data are representative of 2 independent experiments (FIG. 7D and FIG. 7E) or pooled from 2 (FIG. 7B and FIG. 7C) or 3 (FIG. 7A) experiments.

FIG. 8. Proposed mechanism of neutrophil-dependent tumor eradication. Treatment with neutrophil-activating therapy recruits neutrophils to the tumor through TNFR1 signaling and activates the complement AP, generating C5a. C5a signals through C5AR1 in neutrophils and induces neutrophil activation and production of LTB₄, which drives XO activity in the tumor environment. ROS produced by XO induce oxidative damage and death in tumor cells, driving tumor clearance. Tumor-binding antibody contributes to neutrophil killing of tumor cells by inducing ADCC, possibly involving trogocytosis. Although not dependent on adaptive immunity, this process is capable of priming protective immune memory.

FIG. 9A-9E. Neutrophil-activating therapy (NAT) prevents metastases across conditions. (FIG. 9A-9E) 2.5×10⁵ B16F0 tdTomato (B16tdT) cells were injected subcutaneously into the left flank of B6 mice (SQ implantation). One week later, 2.5×10⁵ B16tdT cells were injected into the tail vein of the mice (seeding). (FIG. 9A-9D) Lungs were harvested and imaged 10 days post seeding. Metastases were quantified and compared using 2-way ANOVA with multiple hypothesis correction. (FIG. 9A) Treated mice received intratumoral NAT injection on same day and two days post seeding. (FIG. 9B) Treated mice received intratumoral NAT injection three and five days post seeding. (FIG. 9C) Treated mice received intratumoral NAT injection five and three days prior to seeding. (FIG. 9D) Mice received NAT injection or mock treatment intratumorally three and five days post seeding, or received one dose of NAT intraperitoneally or subcutaneously on the right flank 3 days post seeding. (FIG. 9E) Mice received NAT one and three days post seeding, and lungs were harvested 16 days post seeding. Counts (left) and percent of lung covered by metastasis (right) were quantified and compared using the non-parametric Mann-Whitney U test.

### DETAILED DESCRIPTION

In one aspect, the disclosure provides a combination treatment comprising three agents: a TNF agent; a CD40 agonist; and a tumor-binding antibody. In one aspect, the disclosure provides methods of using the combination therapy in the treatment of cancer. The combination therapy agents and uses thereof are described in more detail below.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of cancer in a mammal, particularly a human, and includes: preventing the cancer and/or symptom(s) from occurring in a subject who may be predisposed to the disease or symptom(s) but has not yet been diagnosed as having it; inhibiting the growth of tumors; inhibiting or arresting progression of cancer; reducing tumor size; slowing or arresting metastatic spread of a tumor; or any other therapeutic effect benefiting a subject with cancer. Those in need of treatment can include those already inflicted (e.g., those with cancer, e.g. those having tumors) as well as those in which prevention is desired (e.g., those with increased susceptibility to cancer; those with pre-cancerous tumors, lesions; those suspected of having cancer, those having genetic predisposition to cancer and/or a family history indicating increased risk of cancer; etc.).

The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, pet animals, and test animals such as dogs, horses, cats, cows, sheep, goats, pigs, rats, mice, monkeys, and others. In a primary embodiment, the mammal is human.

In some embodiments, the individual to be treated is an individual with cancer. As used herein “cancer” includes any form of cancer (e.g., leukemia; acute myeloid leukemia (AML); acute lymphoblastic leukemia (ALL); lymphomas; mesothelioma (MSTO); minimal residual disease; solid tumor cancers, e.g., lung, prostate, breast, bladder, colon, ovarian, pancreas, kidney, glioblastoma, medulloblastoma, leiomyosarcoma, and head & neck squamous cell carcinomas, melanomas; etc.), including both primary and metastatic tumors; and the like. In some cases, the individual has recently undergone treatment for cancer (e.g., radiation therapy, chemotherapy, surgical resection, etc.) and are therefore at risk for recurrence. Any and all cancers are suitable cancers to be treated by the subject methods, compositions, and kits.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, and/or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. The term also includes a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, myelomas, etc., and circulating cancers such as leukemias.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

Certain agents utilized in the practice of the invention comprise antibodies or like antigen-binding fragments. As used herein, the term “antibody” encompasses antibodies, such as a monoclonal antibody, a human antibody, a humanized antibody, a non-human antibody, and includes any other antigen-binding moiety, such as an antibody fragment, chimeric antibody, bispecific antibody, and antigen-binding fragment (e.g. Fab fragment, a Fab′2 fragment, a CDR, or a ScFv), and other antibody fragments that retain specificity for the specified antigen. A full length antibody, as contemplated herein, comprises a heavy chain comprising a Fc domain, and adopts the prototypic tetrameric structure.

### Components of the Combination Therapy

TNF Agent. The combination therapy of the invention encompasses one or more TNF agents. As used herein, a TNF agent comprises is Tumor Necrosis Factor (TNF) or a functional equivalent thereof, i.e., a composition which mimics or functionally recapitulates one or more TNF physiological activities, for example activation of the TNFR1 or TNFR2 receptors. TNF, as used herein encompasses human TNF, engineered variants thereof, and other forms thereof. Examples of TNF agents include but are not limited to a TNF mimetic, a TNF analog, a recombinant TNF, a TNF receptor 1 (TNFR1) agonist, a TNFR1 ligand, an anti-TNFR1 antibody, and a conjugated molecule of one or more of the previously mentioned examples.

In some embodiments, the TNF agent, e.g. a recombinant TNF. In some embodiments, the dose of the TNF agent is, or is physiologically equivalent to at least or about 500 ng, at least or about 600 ng, at least or about 700 ng, at least or about 800 ng, at least or about 900 ng, at least or about 1000 ng, at least or about 1100 ng, at least or about 1200 ng, at least or about 1300 ng, at least or about 1400 ng, at least or about 1500 ng, at least or about 1600 ng, at least or about 1700 ng, at least or about 1800 ng, at least or about 1900 ng, at least or about 2000 ng, at least or about 2500 ng, at least or about 3000 ng, at least about 3500 ng, at least about 4000 ng, at least about 4500 ng, at least about 5000 ng, at least about 5500 ng, at least about 6000 ng, at least about 6500 ng, at least about 7000 ng, at least about 7500 ng, at least about 8000 ng, at least about 8500 ng, at least about 9000 ng, at least about 9500 ng, at least about 10000 ng, or greater of TNF. In some cases, the TNF used with the above dose(s) has an ED₅₀ that is in a range of from 0.5-3 pg/ml (e.g., in an L929 cytotoxicity assay).

In some cases, the above doses are per 50 μl (e.g., 1000 ng (which is 1 μg) is in some cases per 50 μl, which would be equivalent to 20 μg/ml, also 20 ng/μl). As such, in some cases, the dose of the TNF agent is at least or about 10 μg/ml, at least or about 20 μg/ml, at least or about 30 μg/ml, at least or about 40 μg/ml, at least or about 50 μg/ml. In some cases, the dose of the TNF agent is at least 10 μg/ml. In some cases, the dose of the TNF agent is at least 20 μg/ml. In some cases, the dose of the TNF agent is in a range of from 10-100 μg/ml (e.g., 10-90, 10-80, 10-70, 10-60, 10-50, 10-40, 15-100, 15-90, 15-80, 15-70, 15-60, 15-50, 15-40, 20-100, 20-90, 20-80, 20-70, 20-60, 20-50, or 20-40 μg/ml). In some cases, the dose of the TNF agent is in a range of from 15-30 μg/ml. In some cases, the dose of the TNF agent is in a range of from 20-30 μg/ml. In some cases, the dose of the TNF agent is in a range of from 15-25 μg/ml.

CD40 Agent. The combination therapy of the invention encompasses one or more CD40 agents. A CD40 agent, or Cluster of Differentiation 40 agent, is any agent that induces CD40 receptor activation. Examples of CD40 agents include but are not limited to an anti-CD40 agnostic antibody, a CD40 receptor ligand, a recombinant CD40 receptor ligand, a CD40 receptor ligand analog, and a CD40 receptor ligand mimetic.

In some embodiments, the CD40 agent is a CD40 agnostic antibody, for example, an antibody, that, by its binding to CD40, replicates the activity of the native CD40 ligand (CD40L) and activates CD40 signaling pathways. Exemplary CD40 agonist antibodies include selicrelmab, APX005M, ChiLob 7/4, ADC-1013, Sea-CD40, and CDX1140. An example CD40 agonist antibody that activates mouse CD40 is clone FGK4.5 (Bio X Cell, Lebanon, NH).

Tumor-binding Antibody. The treatment of the invention further comprises one or more tumor-binding antibodies. A “tumor-binding antibody,” as used herein, means an antibody, as defined herein, wherein the antibody has binding affinity for a tumor antigen. In some embodiments, the antigen is a tumor-specific antigen, a cancer-specific antigen, a tumor-enriched antigen, a cancer enriched antigen, etc. The antigen can be any antigen expressed by a cancer cell for example, an antigen that is enriched in a cancer cell relative to other cells, or antigen that is unique to a cancer cell, a neoantigen, etc.

Exemplary antigens include one or more of the following proteins (or their orthologs, e.g., human orthologs) (accession identifier is in parentheses): tyrosinase-related protein 1 (TYRP1; also known as glycoprotein 75 (gp75) and MEL-5)(P17643), ATP5I (Q06185), OAT (P29758), AIFM1 (Q9Z0X1), AOFA (Q64133), MTDC (P18155), CMC1 (Q8BH59), PREP (Q8K411), YMEL1 (088967), LPPRC (Q6PB66), LONM (Q8CGK3), ACON (Q99KI0), ODO1(Q60597), IDHP (P54071), ALDH2 (P47738), ATPB (P56480), AATM (P05202), TMM93 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (035887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (055143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (055222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433), and other cancer antigens known in the art.

Exemplary cancer antigen binding antibodies include, for example: clone TA99, Amivantamab, Atezolizumab, Avelumab, Belantamab mafodotin, Bevacizumab, Blinatumomab, Brentuximab vedotin, Capromab, CatumaxomabW, Cemiplimab, Cetuximab, Daratumumab, Dinutuximab, Dostarlimab, Durvalumab, Elotuzumab, Ertumaxomab, Etaracizumab, Glofitamab, Inebilizumab, Inotuzumab ozogamicin, Ipilimumab, Isatuximab, Margetuximab, Mogamulizumab, Moxetumomab pasudotox, Necitumumab, Nimotuzumab, Nivolumab, Olaratumab, Panitumumab, Pembrolizumab, Pertuzumab, Polatuzumab vedotin, Racotumomab, Ramucirumab, Retifanlimab, Sacituzumab govitecan, Siltuximab, Talquetamab, Teclistamab, Trastuzumab, Trastuzumab duocarmazine, Trastuzumab emtansine, and Tremelimumab.

In some implementations, the cancer-antigen binding antibody comprises a polyclonal antibody composition, comprising two or more antibodies that have different binding specificities (i.e., bind to different epitopes of the same target, bind to different target antigens, etc.),

### Combination Therapy Compositions and Methods

In one aspect, the scope of the invention comprises a composition comprising: one or more TNF Agents; one or more CD40 Agents; and one or more tumor-binding antibodies. In one implementation, the scope of the invention comprises a kit, wherein the kit comprises one or more TNF Agents; one or more CD40 Agents; and one or more tumor-binding antibodies.

The agents of the combination therapy may be formulated in one or more pharmaceutical compositions. As used herein, a pharmaceutical composition comprises one or more agents of the combination therapy and may further comprise any number of additional compositions of matter, including excipients, carriers, diluents, release formulations, drug delivery or drug targeting vehicles, as well as additional active therapeutic agents. The pharmaceutical compositions of the invention may be formulated to be compatible with the selected route of administration, for example, intratumoral injection.

The pharmaceutical compositions of the invention may comprise one or more drug delivery compositions. Drug delivery compositions encompass any moieties, materials, or other compositions of matter that facilitate the delivery, retention, and release of the combination therapy agents. Such pharmaceutical compositions may encompass any form of combination, including functionalization of the agents with the delivery composition, conjugation of the agents to the delivery composition; admixture of agents with the delivery composition; encapsulation or infusion of agents within the delivery composition, or any other combination. Exemplary carriers include: liposomes; extracellular vesicles or synthetic mimetics thereof, such as exosomes, microspheres, such as poly(lactic-co-glycolic acid) (PLGA) microspheres; and other drug delivery nanoparticles such as PLGA-PEG nanoparticles, alginate or chitosan nanoparticles, silica nanoparticles, and iron oxide nanoparticles. In one embodiment the pharmaceutical composition comprises or is incorporated within an implant, for example, a drug-eluting implant placed within the target region, for example, polymeric drug-eluting wafers, injectable hydrogels, implantable hydrogel scaffolds, and other drug-eluting implants known in the art.

The pharmaceutical compositions of the invention may be formulated in any number of dosage forms. Exemplary dosage forms include: liquid solutions; sachets, capsules, or tablets, each containing a predetermined amount of the active ingredient, as solids or granules; suspensions in a liquid; emulsions; aqueous and non-aqueous solutions; isotonic sterile injection solutions; compositions stored in a freeze-dried, lyophilized condition; and other dosage forms known in the art.

In various embodiments, the combination therapy comprises one or more TNF agents, one or more CD40 Agonists, and one or more tumor-binding antibodies. In one implementation, the three components are included in a single formulation. In one implementation, two of the components are included in a single formulation and the third component is formulated and packaged separately. In one implementation, each of the three components are formulated and packaged separately.

Administration. The agents of the combination therapy may be administered by any route, including systemic routes, such as intravenously. In the cancer treatments of the invention, the function of the combination therapy is to modulate the tumor microenvironment to induce neutrophil recruitment and activation. Accordingly, in a primary implementation, the administration will be by intratumoral or peritumoral injection, as known in the art. Methods of intratumoral or peritumoral administration have been developed for cancers at different sites in the body, for example, as reviewed in Aznar et al., 2017. Intratumoral Delivery of Immunotherapy Act Locally, Think Globally, J Immunol (2017) 198 (1): 31-39; and De Lombaerde et al., 2021, Delivery routes matter: Safety and efficacy of intratumoral immunotherapy, Biochimica et Biophysica Acta (BBA)—Reviews on Cancer, Volume 1875, Issue 2.

However, in some cases, the route of administration will be subcutaneous (e.g., in some cases for the purpose of preventing metastasis). In some such cases, the primary tumor is inaccessible or has already been removed by surgery or radiation, and yet administration, e.g., systemic administration such as subcutaneous, can still function to prevent metastasis.

The disclosure provides the co-administration of a TNF agent, a CD40 agent, and a tumor-binding antibody, to a subject in need of treatment. The terms “co-administration” and “in combination” include the administration of two or more therapeutic agents either simultaneously, concurrently or sequentially within no specific time limitations. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, and the second agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a third therapeutic agent.

In one implementation, the three components of the combination therapy are present in a single pharmaceutical composition for simultaneous administration. In one implementation, the three components are present in separate pharmaceutical compositions that are mixed at the time of administration and then administered together. In one implementation, the components are present in separate pharmaceutical compositions and re administered separately. In one implementation, the three components are administered substantially simultaneously. In one implementation, the one or more of the three components are administered in a particular order, at different times.

### Exemplary Embodiments

In one embodiment, the disclosure provides a composition comprising: a TNF agent; a CD40 agent; and a tumor-binding antibody.

In one embodiment the scope of the invention encompasses a kit comprising: a TNF agent; a CD40 agent; and a tumor-binding antibody.

In one embodiment, the disclosure provides a composition comprising: a TNF agent; a CD40 agent; and a tumor-binding antibody, for use in a method of treating cancer in a subject.

In one embodiment, the disclosure provides a method of manufacturing a medicament comprising: a TNF agent; a CD40 agent; and a tumor-binding antibody, for use in a method of treating cancer in a subject.

In one embodiment, the disclosure provides a method of treating cancer in a subject, by co-administering to the subject a TNF agent; a CD40 agent; and a tumor-binding antibody. In one embodiment, the agents are substantially simultaneously.

In various embodiments of the foregoing, the TNF agent is TNF, for example, recombinant human TNF. In various embodiments, the TNF is administered in a dose of at least 500 ng, at least 750 ng, at least 1000 ng, at least 1500 ng, or at least 2000 ng.

In various embodiments of the foregoing, the CD40 agent is a CD40 agonist antibody. In various embodiments, the CD40 agent may be selected from the group consisting of selicrelmab, APX005M, ChiLob 7/4, ADC-1013, Sea-CD40, and CDX1140.

In various embodiments of the foregoing, the tumor-binding antibody is selected from the group consisting of Amivantamab, Atezolizumab, Avelumab, Belantamab mafodotin, Bevacizumab, Blinatumomab, Brentuximab vedotin, Capromab, Catumaxomab^{I}, Cemiplimab, Cetuximab, Daratumumab, Dinutuximab, Dostarlimab, Durvalumab, Elotuzumab, Ertumaxomab, Etaracizumab, Glofitamab, Inebilizumab, Inotuzumab ozogamicin, Ipilimumab, Isatuximab, Margetuximab, Mogamulizumab, Moxetumomab pasudotox, Necitumumab, Nimotuzumab, Nivolumab, Olaratumab, Panitumumab, Pembrolizumab, Pertuzumab, Polatuzumab vedotin, Racotumomab, Ramucirumab, Retifanlimab, Sacituzumab govitecan, Siltuximab, Talquetamab, Teclistamab, Trastuzumab, Trastuzumab duocarmazine, Trastuzumab emtansine, and Tremelimumab.

In various embodiments of the foregoing, the cancer is selected from the group consisting of lung, prostate, breast, bladder, colon, ovarian, pancreas, kidney, liver, glioblastoma, medulloblastoma, leiomyosarcoma, head & neck squamous cell carcinomas, melanomas, carcinomas; soft tissue tumors; sarcomas; teratomas; melanomas; leukemias; lymphomas; and brain cancers, including minimal residual disease, and including both primary and metastatic tumors; cancers of the breast, pancreas, lung, prostate, and colon comprising adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; Carcinomas of the prostrate, pancreas, colon, brain lung, breast, skin, and metastases of the foregoing.

Examples of embodiments include, but are not limited to:
1. A composition comprising: a TNF agent; a CD40 agent; and a tumor-binding antibody.
2. A kit comprising: a TNF agent; a CD40 agent; and a tumor-binding antibody.
3. The composition of 1 or the kit of 2, wherein the TNF agent is TNF.
4. The composition of 1 or the kit of 2, wherein the CD40 agent is a CD40 agonist antibody.
5. The composition or kit of 4, wherein the CD40 agent is selected from the group consisting of selicrelmab, APX005M, ChiLob 7/4, ADC-1013, Sea-CD40, and CDX1140.
6. The composition of 1 or the kit of 2, wherein the tumor-binding antibody is selected from the group consisting of Amivantamab, Atezolizumab, Avelumab, Belantamab mafodotin, Bevacizumab, Blinatumomab, Brentuximab vedotin, Capromab, Catumaxomab^{I}, Cemiplimab, Cetuximab, Daratumumab, Dinutuximab, Dostarlimab Durvalumab, Elotuzumab, Ertumaxomab, Etaracizumab, Glofitamab, Inebilizumab, Inotuzumab ozogamicin, Ipilimumab, Isatuximab, Margetuximab, Mogamulizumab, Moxetumomab pasudotox, Necitumumab, Nimotuzumab, Nivolumab, Olaratumab, Panitumumab, Pembrolizumab, Pertuzumab, Polatuzumab vedotin, Racotumomab, Ramucirumab, Retifanlimab, Sacituzumab govitecan, Siltuximab, Talquetamab, Teclistamab, Trastuzumab, Trastuzumab duocarmazine, Trastuzumab emtansine, and Tremelimumab.
7. A method of treating cancer in a subject in need of treatment therefor, comprising administering to the subject: a TNF agent, a CD40 agent, and a tumor-binding antibody.
8. The method of 7, wherein the cancer is selected from the group consisting of lung, prostate, breast, bladder, colon, ovarian, pancreas, kidney, liver, glioblastoma, medulloblastoma, leiomyosarcoma, head & neck squamous cell carcinomas, melanomas, carcinomas; soft tissue tumors; sarcomas; teratomas; melanomas; leukemias; lymphomas; and brain cancers, including minimal residual disease, and including both primary and metastatic tumors; cancers of the breast, pancreas, lung, prostate, and colon comprising adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; Carcinomas of the prostrate, pancreas, colon, brain lung, breast, skin, and metastases of the foregoing.
9. The method of 7, wherein the TNF agent is TNF.
10. The method of 7, wherein the CD40 agent is a CD40 agonist antibody.
11. The method of 10, wherein the CD40 agnostic antibody is selected from the group consisting of selicrelmab, APX005M, ChiLob 7/4, ADC-1013, Sea-CD40, and CDX1140.
12. The method of 7, wherein the tumor-binding antibody is selected from the group consisting of Amivantamab, Atezolizumab, Avelumab, Belantamab mafodotin, Bevacizumab, Blinatumomab, Brentuximab vedotin, Capromab, Catumaxomab^{I}, Cemiplimab, Cetuximab, Daratumumab, Dinutuximab, Dostarlimab, Durvalumab, Elotuzumab, Ertumaxomab, Etaracizumab, Glofitamab, Inebilizumab, Inotuzumab ozogamicin, Ipilimumab, Isatuximab, Margetuximab, Mogamulizumab, Moxetumomab pasudotox, Necitumumab, Nimotuzumab, Nivolumab, Olaratumab, Panitumumab, Pembrolizumab, Pertuzumab, Polatuzumab vedotin, Racotumomab, Ramucirumab, Retifanlimab, Sacituzumab govitecan, Siltuximab, Talquetamab, Teclistamab, Trastuzumab, Trastuzumab duocarmazine, Trastuzumab emtansine, and Tremelimumab.
13. The method of 7, wherein the TNF agent; the CD40 agent; and the tumor-binding antibody are co-administered substantially simultaneously.
14. The method of 7, wherein the TNF agent; the CD40 agent; and the tumor-binding antibody are administered intratumorally or peritumorally.
15. The method of any one of 7-13, wherein said administering comprises systemic administration.
16. The method of any one of 7-13, wherein said administering comprises subcutaneous administration.

### EXAMPLES

### Example 1. Neutrophil-Activating Therapy for the Treatment of Cancer

Results. Neutrophil-activating therapy recruits activated neutrophils to the tumor. To determine if neutrophils might be harnessed therapeutically, we investigated how the tumor microenvironment could be modulated to optimally recruit neutrophils and activate their cytotoxic function. When we assessed the impact of various cytokines injected intratumorally in B16 melanoma, tumor necrosis factor (TNF) notably induced robust recruitment of neutrophils and upregulated neutrophil surface molecules consistent with activation³⁴⁻³⁶ in a dose-dependent manner. However, TNF monotherapy failed to clear tumors in most mice.

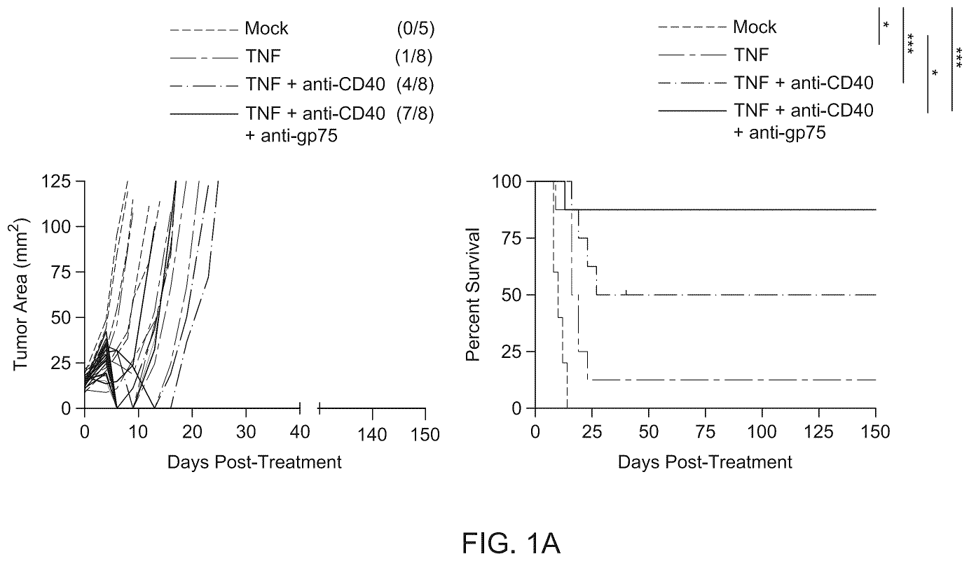

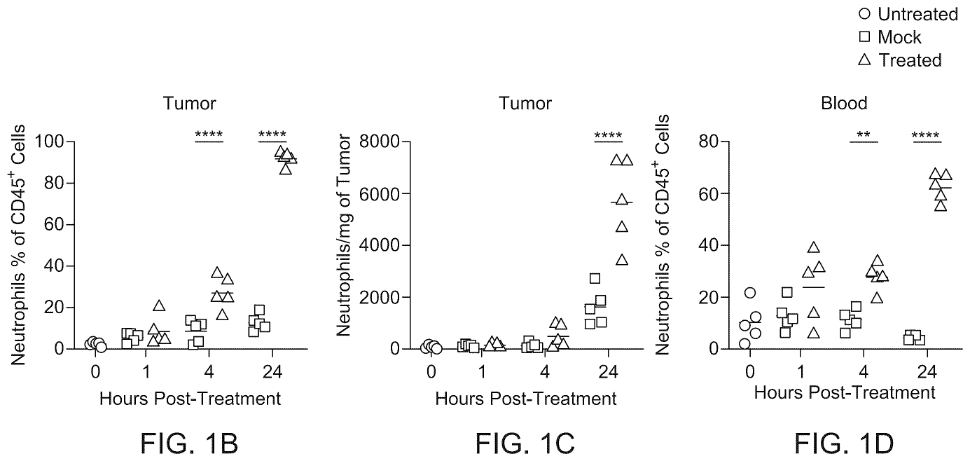

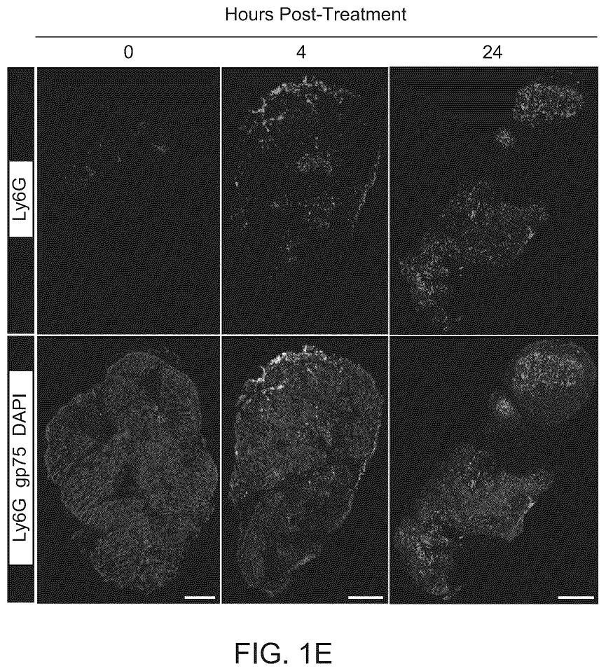

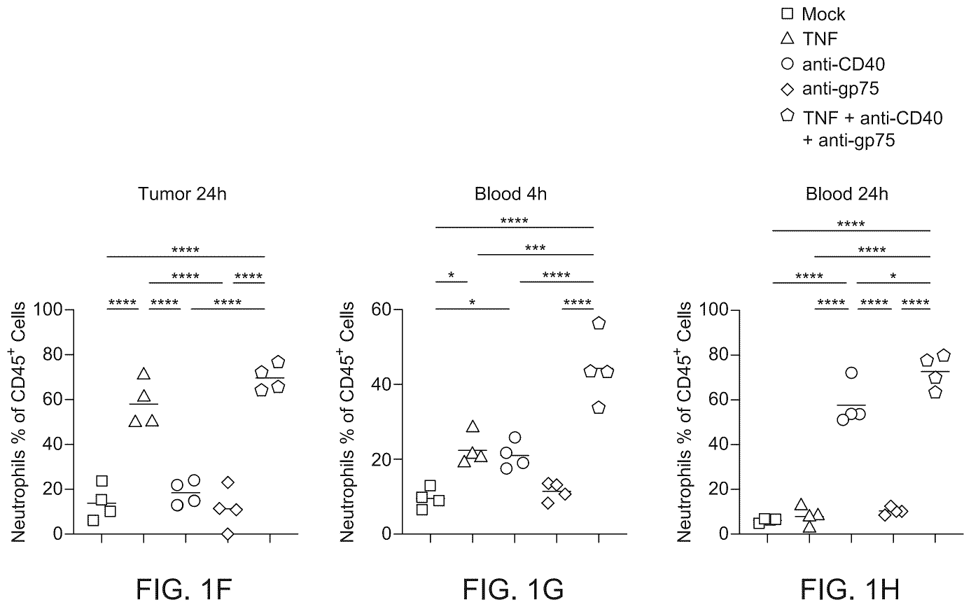

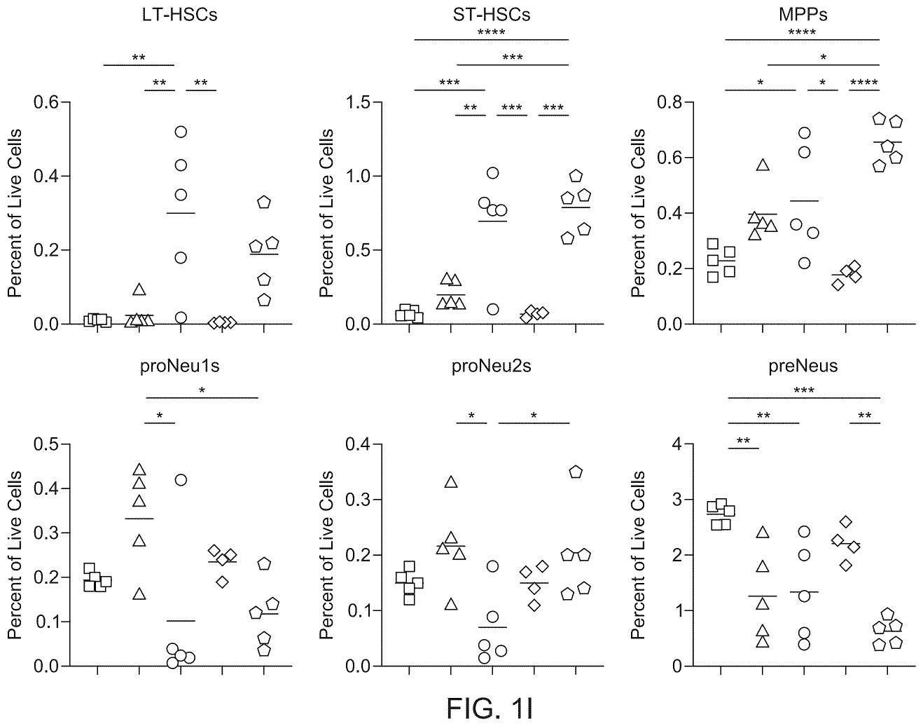

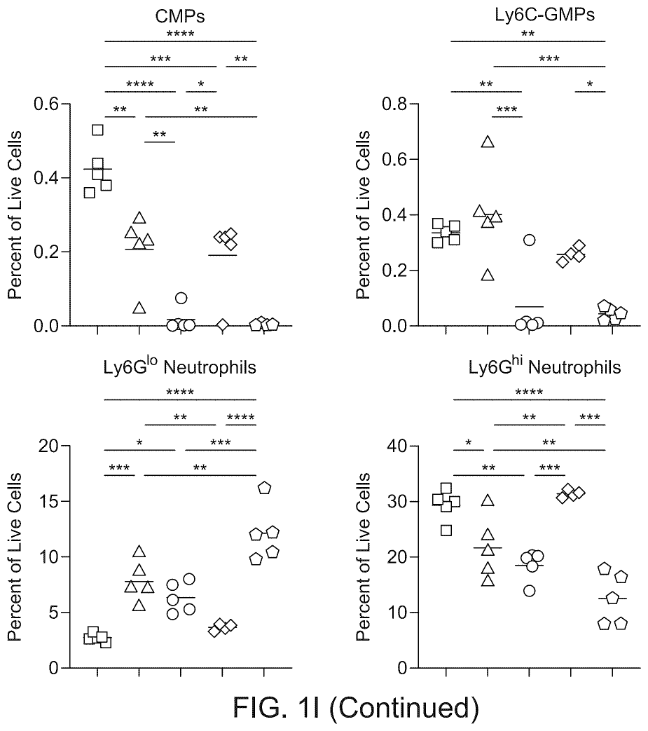

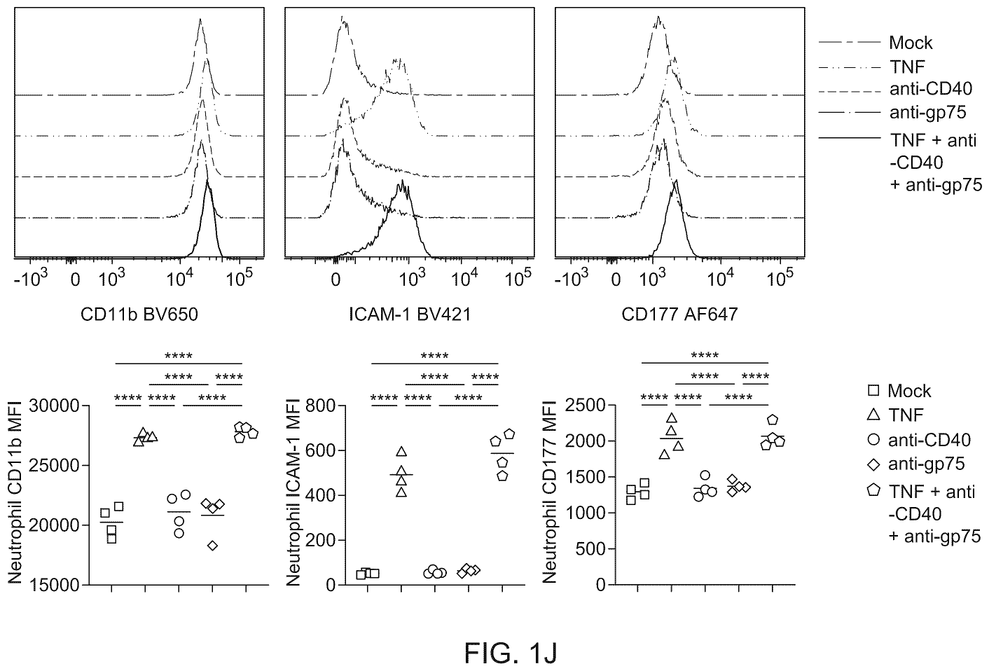

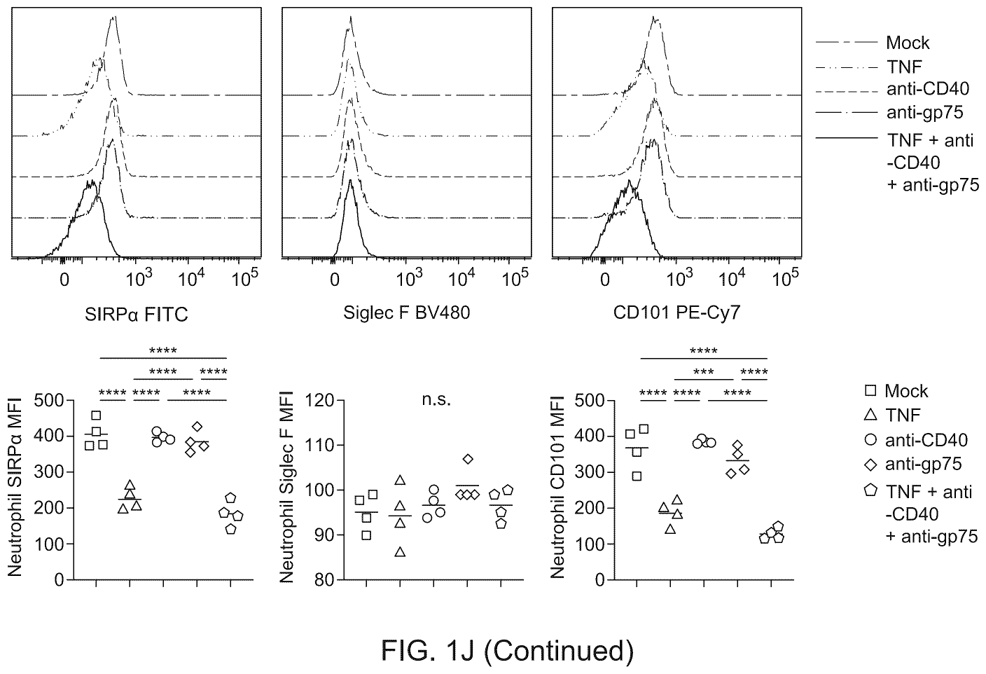

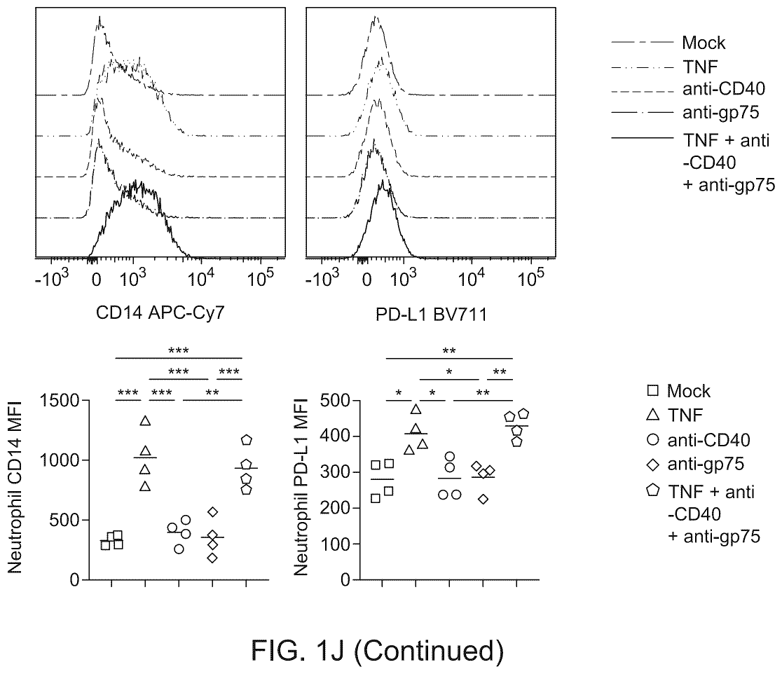

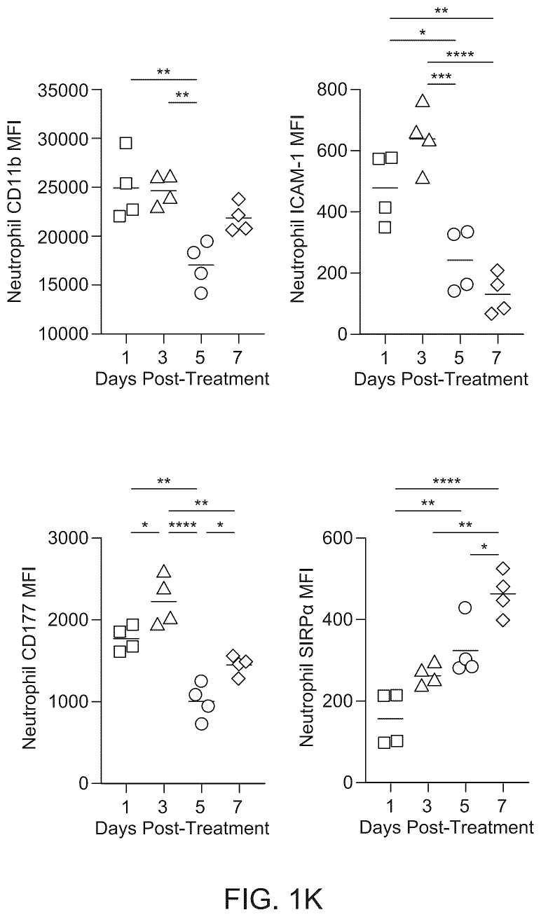

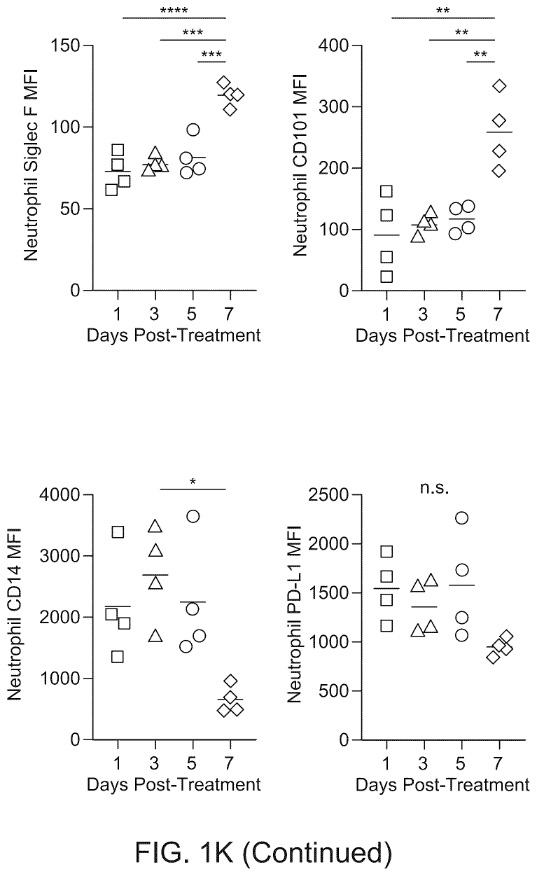

Given the promising neutrophil infiltration and activation induced by TNF but the failure to clear tumors, we sought complementary agents capable of enabling neutrophil-mediated tumor clearance in combination with TNF. As cluster of differentiation 40 (CD40) agonists can activate neutrophils and promote neutrophil cytotoxicity,³⁷ we evaluated the effect of an agonistic anti-CD40 monoclonal antibody. As neutrophils are also potent mediators of antibody-dependent cellular cytotoxicity (ADCC) through ligation of their Fc receptors,^{2,32} we also tested a mAb targeting the melanoma-associated antigen gp75. Strikingly, intratumoral treatment of tumors with two doses of 1 μg TNF+100 μg anti-CD40+100 μg anti-gp75 two days apart induced durable clearance of B16 melanoma tumors (FIG. 1A). In contrast, treatment with only one or two of these components failed to achieve the same frequency of tumor clearance. Mice treated with TNF+anti-CD40+anti-gp75 transiently lost a small amount of weight but rapidly recovered within two days of the second treatment. Additionally, blood chemistry analysis one week after treatment completion and 60 days post-treatment revealed essentially normal liver and renal function.

Mice treated with the full three-component therapy consisting of TNF, anti-CD40, and tumor-binding antibody, hereafter referred to as neutrophil-activating therapy, exhibited rapid recruitment of neutrophils to tumors (FIGS. 1B and 1C), and neutrophils expanded in the blood with similar kinetics (FIG. 1D). Treated neutrophils infiltrated throughout B16 tumors and were not merely confined to the periphery (FIG. 1E). Evaluation of the impact of individual treatment components revealed that neutrophil recruitment to tumors was primarily induced by TNF (FIG. 1F). Although TNF induced a transient increase in neutrophil frequency in the blood (FIG. 1G), anti-CD40 induced a large and sustained expansion of neutrophils in the blood (FIG. 1H).

Flow cytometry analysis to identify hematopoietic stem cells (HSCs) and progenitors38-40 in the bone marrow 24 h after neutrophil-activating therapy revealed an increase in the frequencies of HSCs and multipotent progenitors (MPPs) (FIG. 1I). Although the full neutrophil-activating therapy had a minimal effect on the frequency of common lymphoid progenitors (CLPs), it induced a drastic reduction in common myeloid progenitors (CMPs), granulocyte-monocyte progenitors (GMPs), megakaryocyte-erythrocyte progenitors (MEPs), and common monocyte progenitors (cMoPs), whereas committed neutrophil progenitors (proNeuIs and proNeu2s) were not significantly altered (FIG. 1I). These data indicate the induction of granulopoiesis. In addition, although the frequency in the bone marrow of late neutrophil precursors (preNeus) decreased with therapy, Ly6^{lo} immature neutrophils increased, and Ly6G^{hi} mature neutrophils decreased (FIG. 1I), consistent with an increased differentiation of neutrophils in the bone marrow and a mobilization of mature neutrophils into the blood. Furthermore, the spleen displayed an expansion of neutrophils and similar alterations in the frequencies of HSCs and progenitors, indicating extramedullary granulopoiesis.

In mice lacking TNF receptors (TNFR knockout [KO] mice), treatment with neutrophil-activating therapy failed to recruit or activate neutrophils. Although treatment induced cell death throughout tumors in wild-type (WT) mice, TNFR KO mice had reduced levels of cell death and failed to clear their tumors following treatment. Tumor clearance was mediated through TNF receptor 1 (TNFR1) and was independent of TNFR1 expression on tumor cells. These data demonstrate that TNF signaling in non-tumor cells is essential for neutrophil recruitment, tumor cell killing, and tumor clearance.

Neutrophil-activating therapy induced multiple alterations in the surface phenotype of tumor-infiltrating neutrophils, primarily in response to TNF (FIG. 1J). These neutrophils upregulated CD11b and intercellular adhesion molecule (ICAM)-1, indicating activation or priming,³⁴⁻³⁶ and increased expression of CD177, which has been associated with anti-tumor neutrophils in colon cancer.⁴¹ Therapeutically activated neutrophils also had lower levels of signal regulatory protein-a (SIRPa), a myeloid check-point that inhibits neutrophil ADCC,^{2,32} demonstrating the capacity for enhanced tumor cell killing. Neutrophil-activating therapy did not alter the expression of Siglec F, a marker associated with pro-tumor neutrophils,⁴² which remained low in all treatment conditions. In contrast, treated neutrophils downregulated, but did not lose, expression of CD101 (Figure S2K). CD101-negative neutrophils are more immature and have been reported to correlate with tumor burden.³⁸ Neutrophils in tumors treated with neutrophil-activating therapy also upregulated CD14 and programmed cell death-ligand 1 (PD-L1), which have been reported to identify neutrophils with increased suppressive and reduced tumoricidal activity.^{2,43,44} Neutrophils in the blood exhibited a very similar, but less extreme, pattern of changes in expression of these markers following neutrophil-activating therapy. These alterations in neutrophil phenotype were transient, as neutrophils in both the tumor and blood increasingly reverted toward the phenotype of neutrophils in mock treated mice over the first week post-treatment (FIG. 1K). Neutrophil-activating therapy of other tumors, including Sparkl.4640, a colon carcinoma cell line isolated from a genetically engineered mouse model, and 4T1 mammary carcinoma induced similar neutrophil expansion, recruitment, and activation.

Furthermore, treated tumor-infiltrating neutrophils possessed a mature morphology with hypersegmented nuclei. Consistent with their activated status, they exhibited enhanced production of reactive oxygen species (ROS). Although activated neutrophils can extrude neutrophil extracellular traps (NETs), neutrophils in the tumor and blood of treated mice exhibited no detectable increase in NETotic neutrophils⁴⁵. Altogether, these data indicate that neutrophil-activating therapy induces an acute activation of tumor-infiltrating and circulating neutrophils, which exhibit a unique surface phenotype including markers associated with both anti-tumor and pro-tumor function.

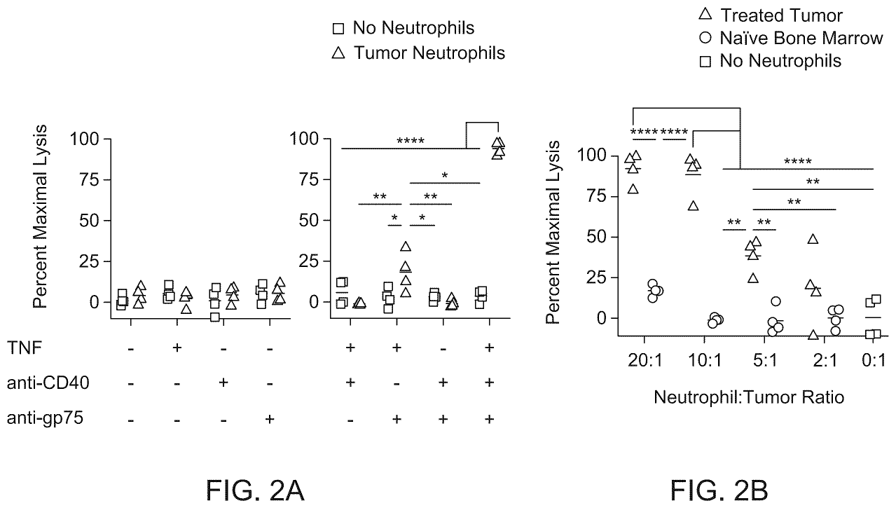

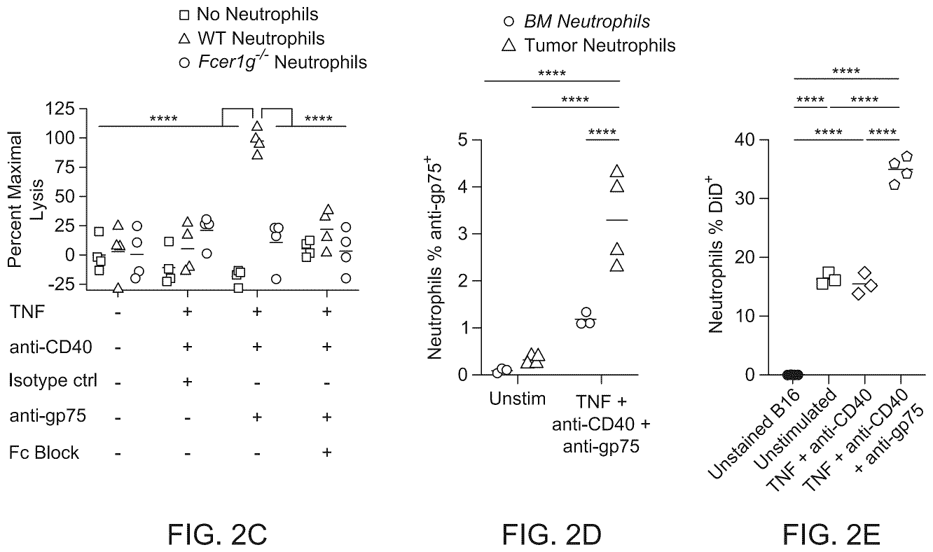

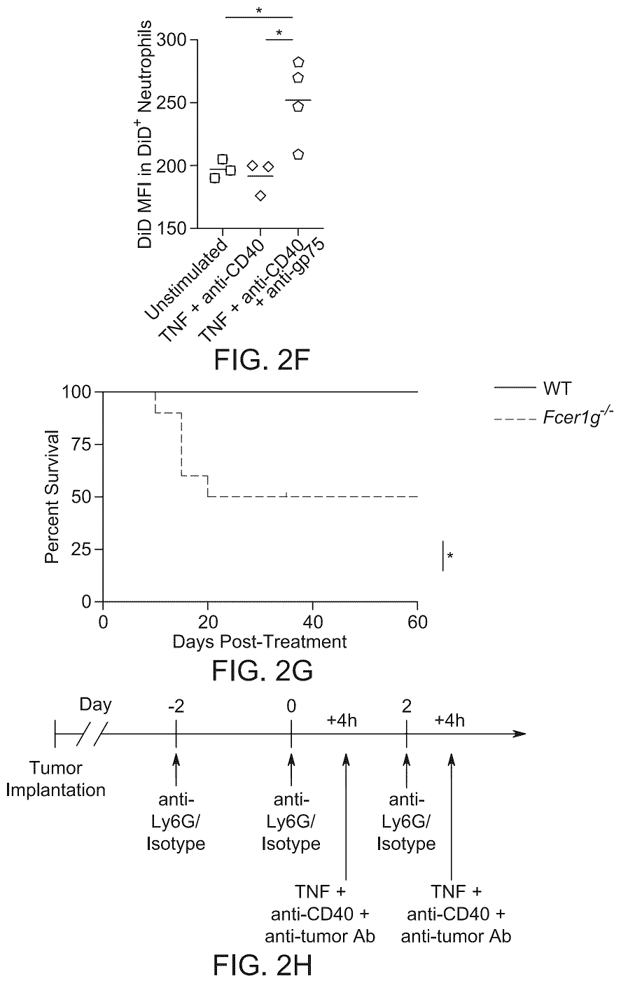

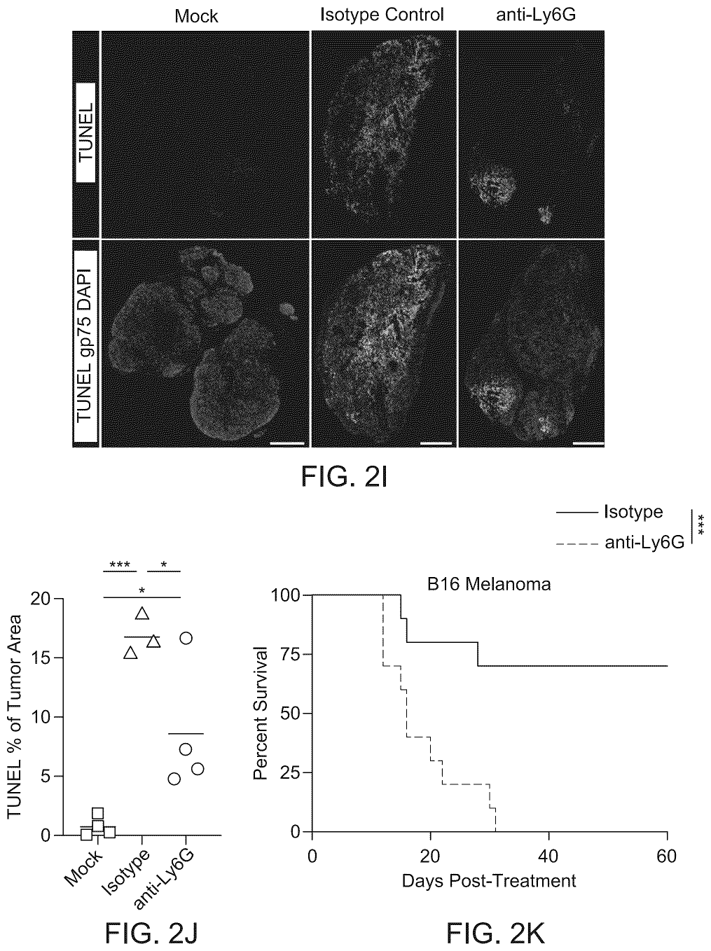

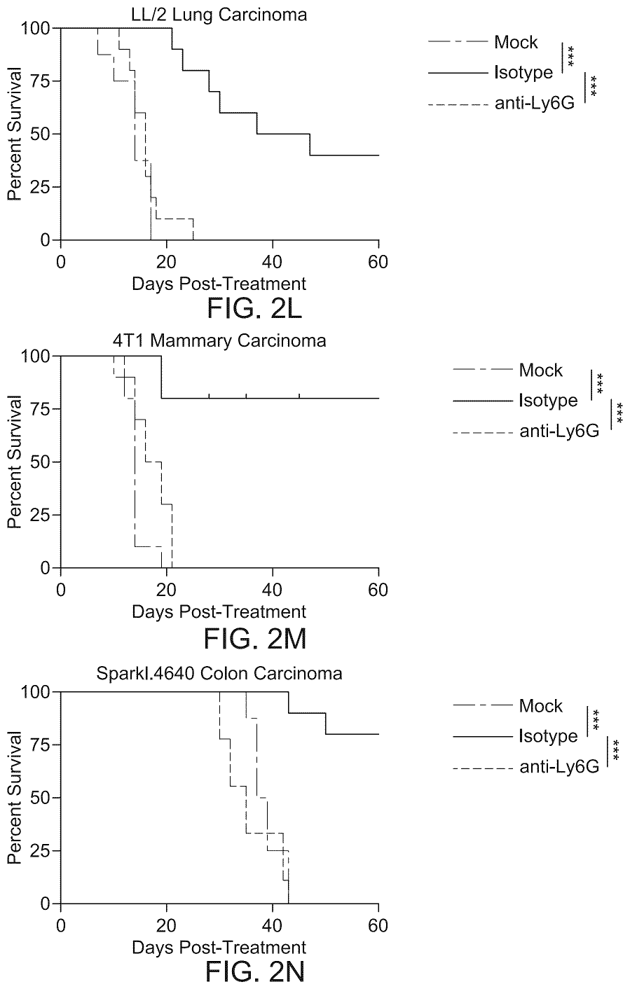

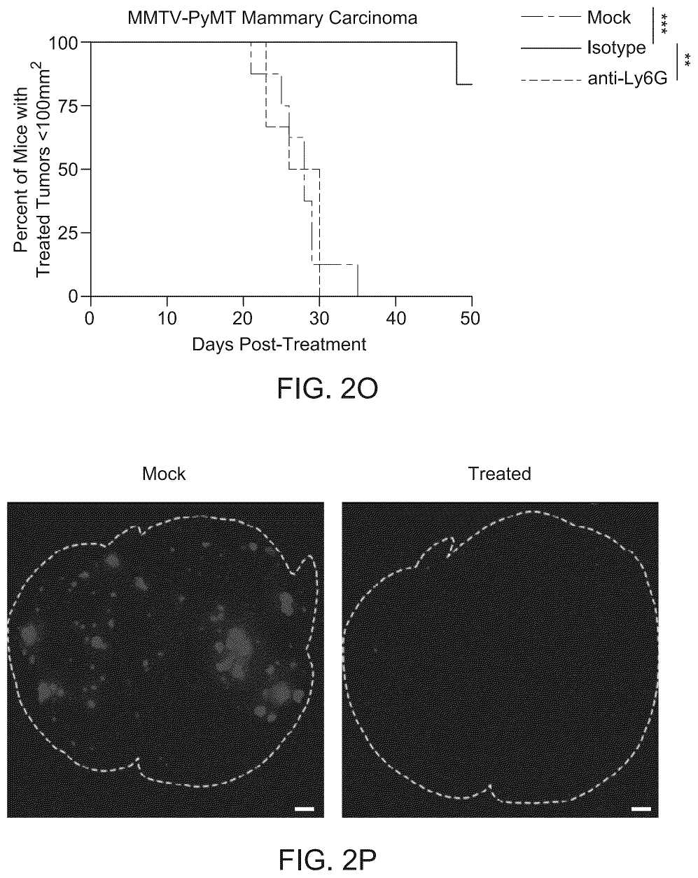

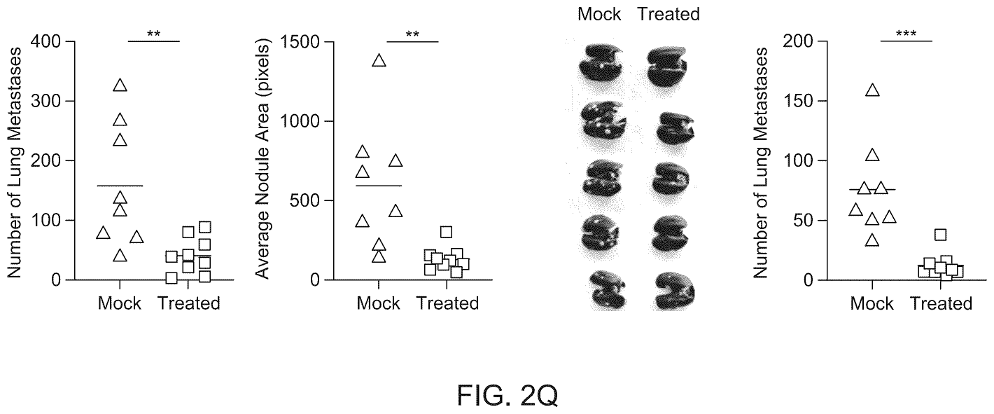

Therapeutically activated neutrophils eradicate multiple tumor types and reduce metastatic seeding. To directly evaluate the anti-tumor activity of neutrophils following neutrophil-activating therapy, neutrophils were isolated from treated B16 tumors and co-cultured with B16 tumor cells ex vivo. Following stimulation ex vivo with neutrophil-activating therapy, these neutrophils mediated potent tumor cell killing (FIG. 2A). In contrast, neutrophils isolated from the bone marrow of naive mice failed to mediate significant tumor cell killing, even after ex vivo stimulation with neutrophil-activating therapy (FIG. 2B). Notably, stimulation with all three components of the therapy was required for maximal cytotoxic function (FIG. 2A). Tumor-infiltrating neutrophils required both anti-gp75 and Fc gamma receptors (FcgRs) to kill B16 tumor cells ex vivo (FIG. 2C), and tumor-infiltrating neutrophils stimulated with neutrophil-activating therapy demonstrated enhanced uptake of anti-gp75 mAb (FIG. 2D) and B16 cell membrane (FIGS. 2E and 2F), showing that antibody-mediated trogocytosis³² may mediate ADCC by these activated neutrophils. Mice lacking functional activating FcgRs (Fcer1g^{/}) exhibited reduced tumor clearance in vivo (FIG. 2G), while neutrophil recruitment and CD11b upregulation were still maintained. B16 tumors that recurred following therapy exhibited reduced levels of the antibody target antigen gp75, suggesting that ADCC may exert a selection pressure against the antibody target antigen. Treatment of mice bearing B16 tumors with heterogeneous expression of cell-surface enhanced green fluorescent protein (EGFP) with TNF+anti-CD40+anti-EGFP induced tumor clearance at a comparable rate as tumors with homogeneous EGFP expression. Altogether, these data suggest that tumor-binding antibody-FcgR interactions enhance in vivo neutrophil killing of tumors through ADCC, but this is not absolutely required for tumor clearance.

To determine the role of therapeutically activated neutrophils in tumor clearance in vivo, neutrophils were depleted using anti-Ly6G antibodies (FIG. 2H). Although neutrophil depletion with anti-Ly6G did not completely remove neutrophils, this protocol blocked treatment-induced neutrophil tumor infiltration and reduced neutrophil expansion in the blood. Similar results were obtained across multiple tumor models.

Depletion of neutrophils with anti-Ly6G mAb prior to treatment markedly reduced cell death within tumors (FIGS. 2I and 2J). Although neutrophil depletion did not alter tumor growth in untreated mice, it completely prevented tumor clearance in response to neutrophil-activating therapy (FIG. 2K), demonstrating a crucial role for neutrophils in tumor eradication.

Treatment with neutrophil-activating therapy enabled neutrophils to clear multiple additional tumor types, including LL/2 lung carcinoma, 4T1 mammary carcinoma, and Sparkl.4640 colon carcinoma (FIGS. 2L-2N). We next tested neutrophil-activating therapy in the MMTV-PyMT model of mammary carcinoma, in which tumors spontaneously develop in multiple breasts nearly simultaneously. Treatment of one tumor per mouse resulted in neutrophil-dependent regression of the treated tumors (FIG. 2O), whereas tumors in untreated breasts did not regress. Tumors later grew out in treated breasts, which could represent either recurrences of the treated tumor or development of additional tumors in the same breast.

To determine whether neutrophil-activating therapy could restrict the growth of distant tumors in the context of metastasis, we injected B16 expressing tdTomato into the tail vein one week after subcutaneous implantation of B16. Treatment of the subcutaneous tumor ten hours after this seeding of the lungs resulted in a substantial reduction in the number and size of lung metastases (FIG. 2P). Furthermore, treatment of orthotopically implanted 4T1 mammary carcinoma with neutrophil-activating therapy reduced the number of spontaneous lung metastases (FIG. 2Q). These data indicate that neutrophil-activating therapy can reduce metastatic seeding, restrict metastatic growth, and/or eliminate metastases.

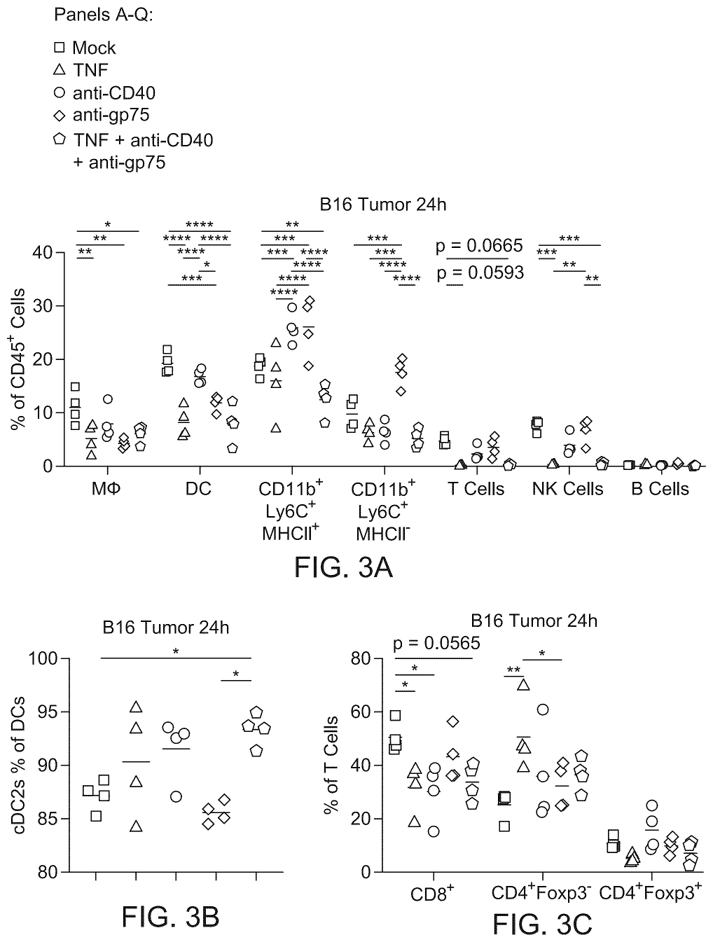

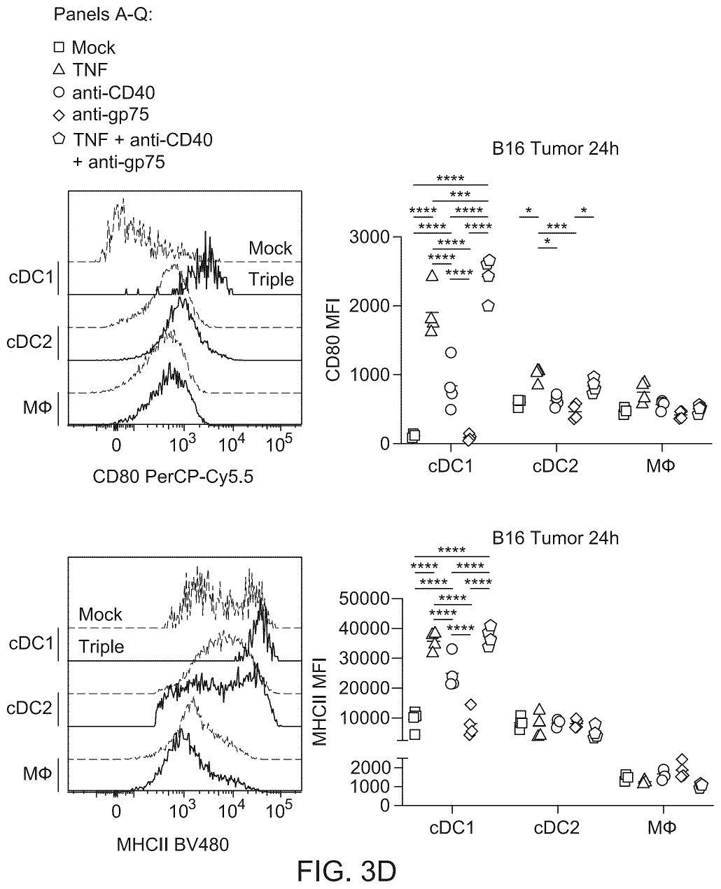

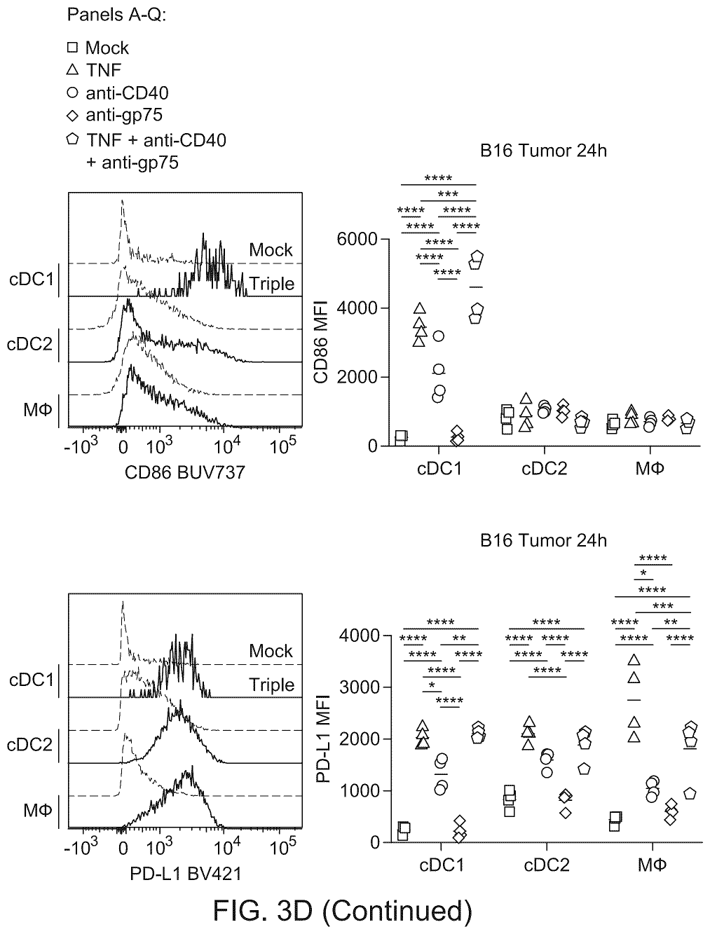

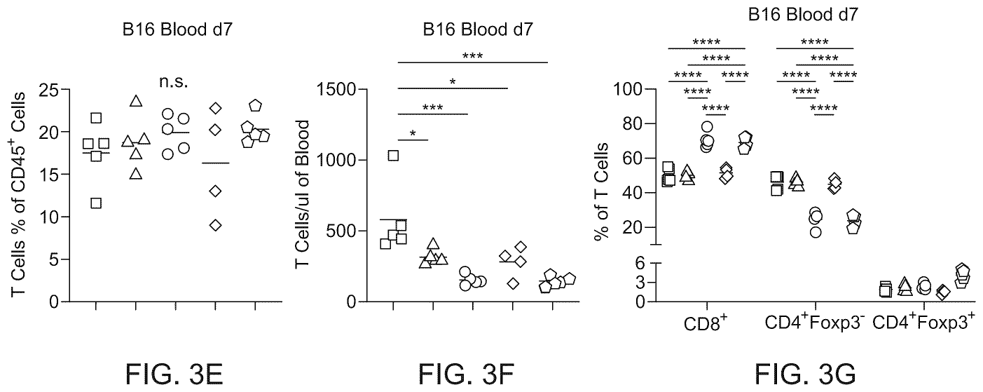

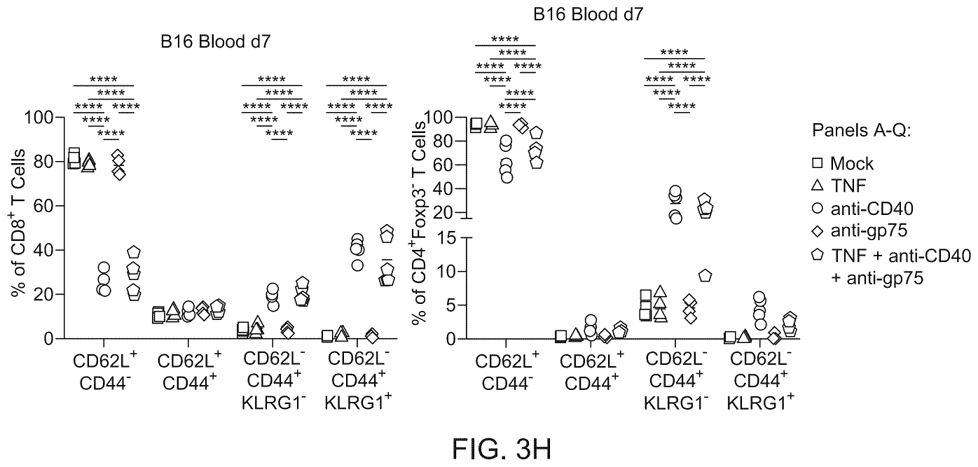

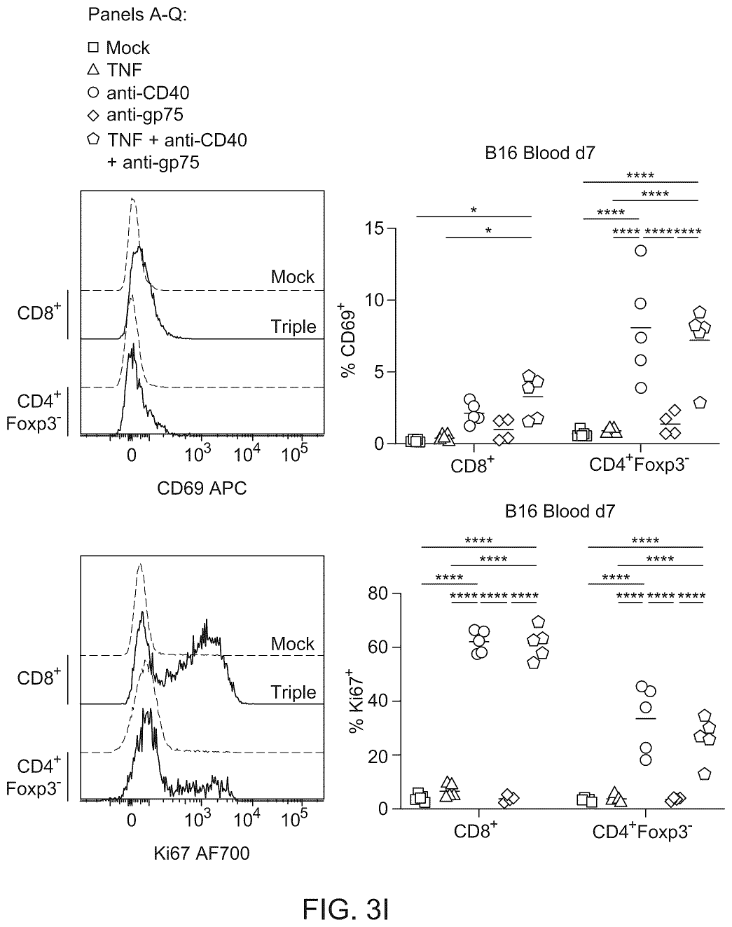

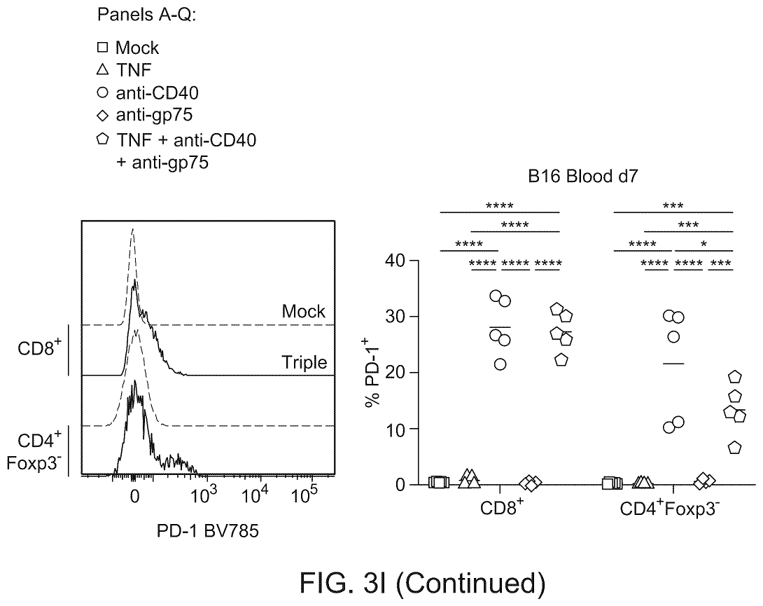

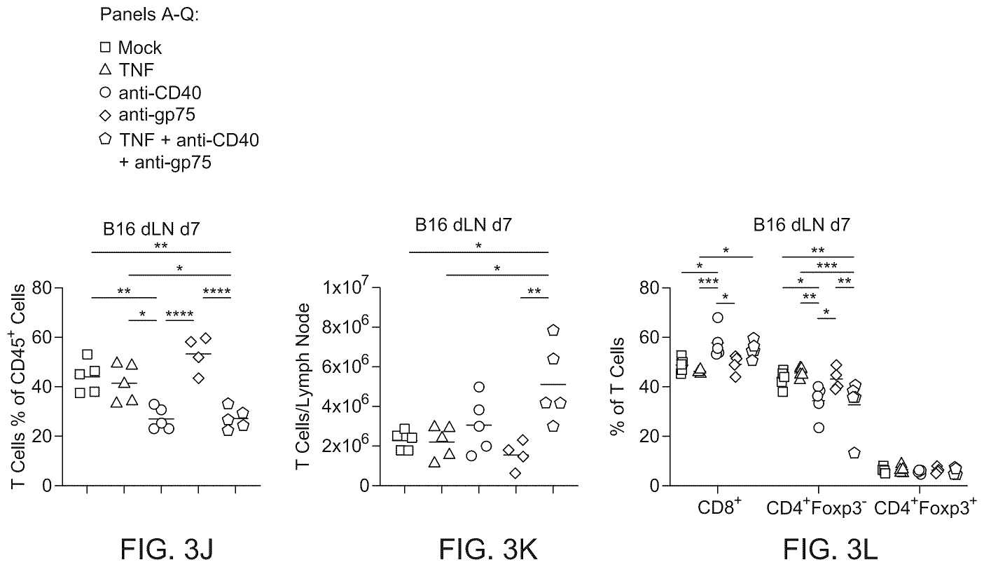

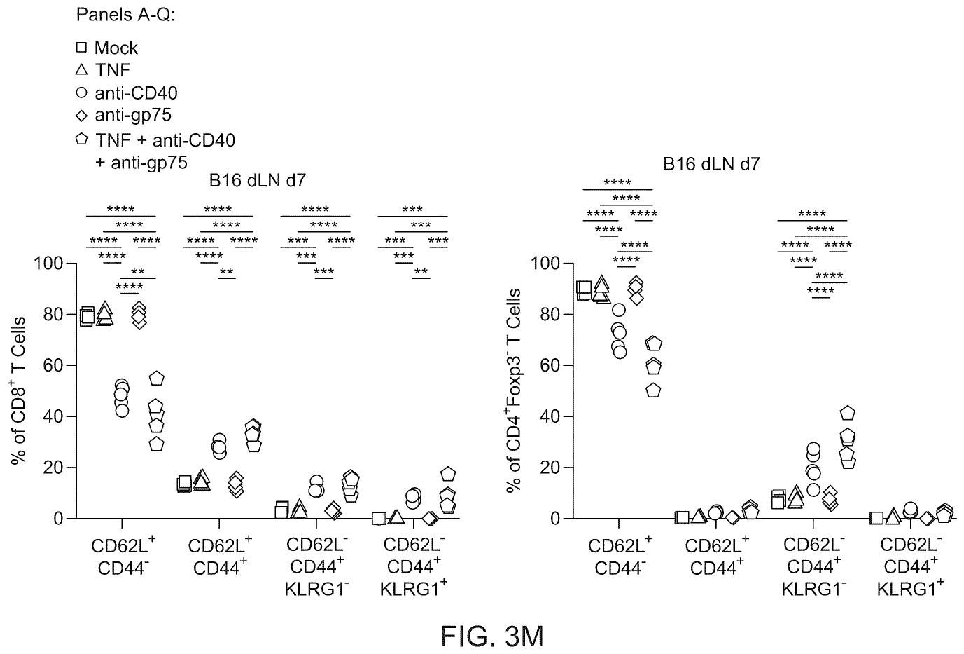

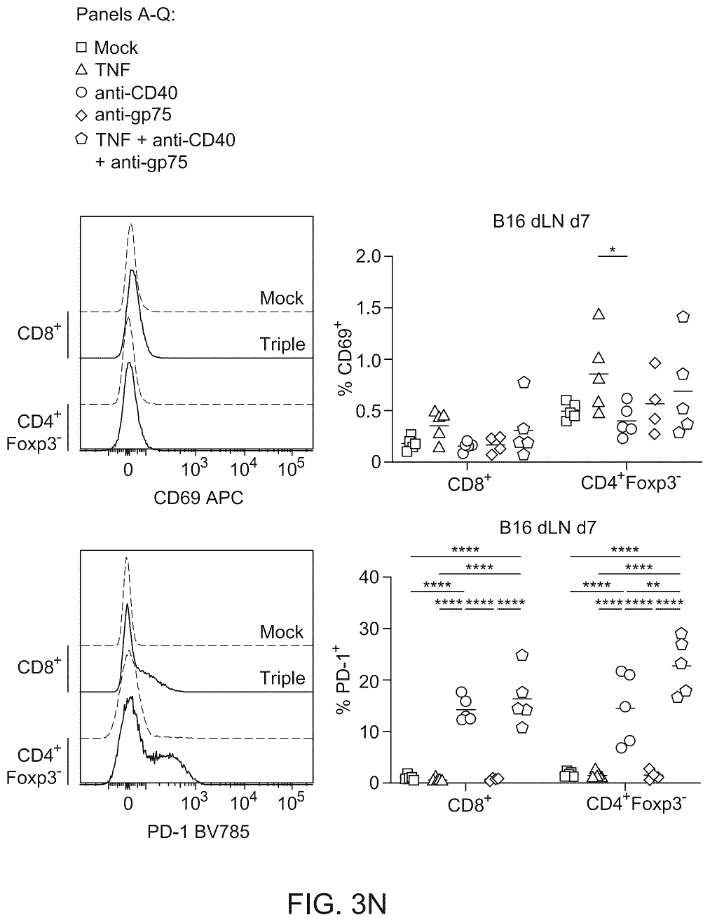

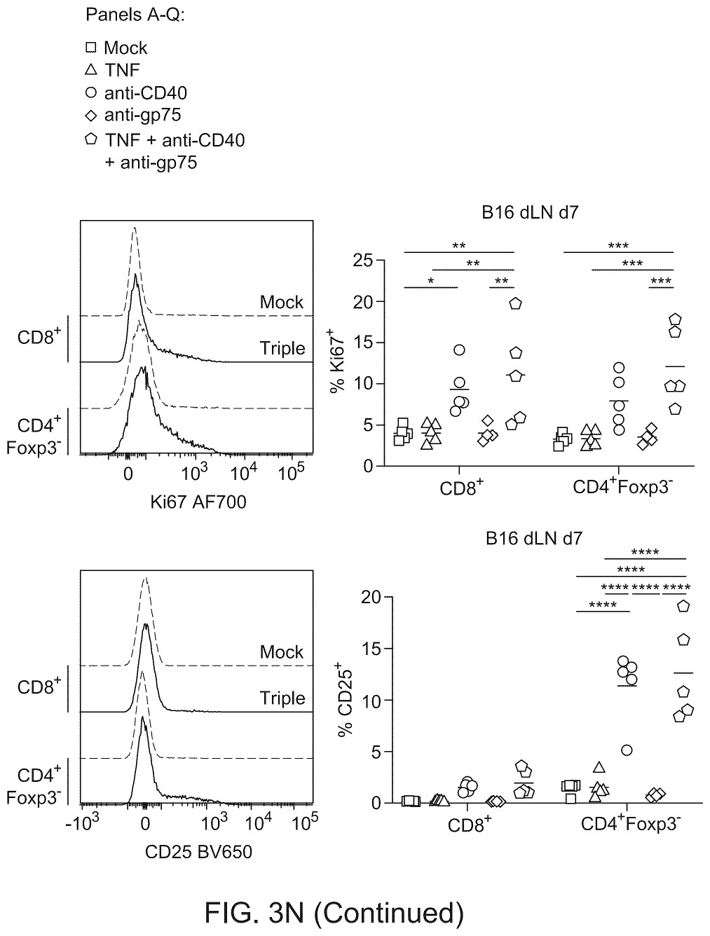

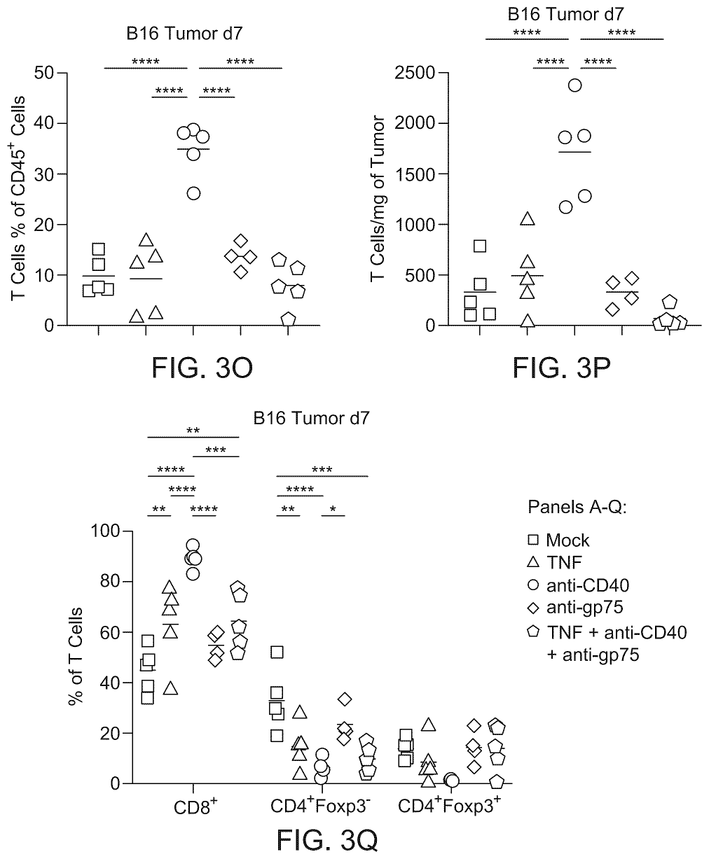

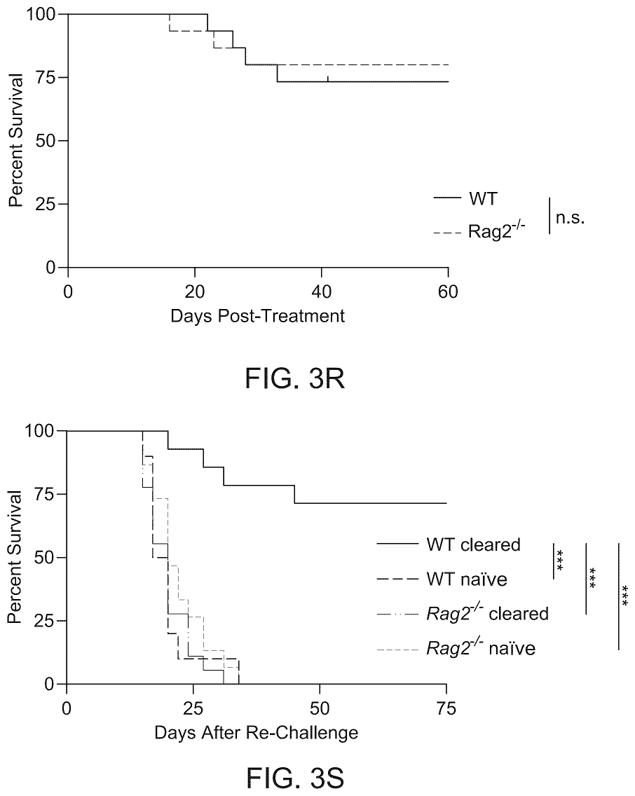

Therapy activates antigen-presenting cells and primesT cell memory. Neutrophil infiltration following treatment was accompanied by a TNF-dependent decrease in multiple other immune cell populations within the tumor (FIG. 3A). Additionally, conventional dendritic cell (cDC) populations were skewed further toward cDC2s (FIG. 3B), and the relative frequency of CD8⁺ T cells decreased slightly after treatment (FIG. 3C). Treatment of TNFR KO mice with neutrophil-activating therapy did not induce the large alterations to immune cell populations seen in treated WT mice. In contrast, treated Fcer1g^{/} mice had alterations in immune subsets closely resembling treated WT mice. The anti-CD40-dependent expansion of neutrophils in the blood (FIG. 1H) was counterbalanced by a decrease in blood B cell frequency, and anti-CD40 increased the proportion of circulating CD4⁺ Forkhead box P3⁻ (Foxp3⁻) T cells at the expense of CD8⁺ T cells. Treatment of 4T1 and Sparkl.4640 tumors with neutrophil-activating therapy elicited similar changes in immune cell populations.

TNF and CD40 agonists are capable of activating antigen-presenting cells (APCs); indeed, both agents induced upregulation of major histocompatibility complex class II (MHCII), CD80, and CD86 on cDC1s in B16 tumors (FIG. 3D), indicating activation. In contrast, there were minimal changes in activation markers in the more abundant cDC2s and macrophages, other than CD80 upregulation in cDC2s (FIG. 3D). Opposing this activation, all three APC subsets upregulated PD-L1 with treatment (FIG. 3D). Macrophage polarization, assessed by the ratio of CD206^{hi} to MHCII^{hi} macrophages, did not change significantly following therapy. Sparkl.4640 and 4T1 tumors treated with neutrophil-activating therapy exhibited mostly similar patterns of APC activation. Altogether, these data indicate that neutrophil-activating therapy induces activation of APCs in the tumor, although this activation is mainly confined to the relatively rare cDC1 subset.

To determine the effects of neutrophil-activating therapy on T cells, we examined blood, tumor-draining lymph nodes (dLNs), and tumors one week after treatment of B16 tumors. Although T cells in the blood did not expand (FIGS. 3E and 3F), there was an anti-CD40-dependent increase in the proportion of CD8⁺ T cells (FIG. 3G), as well as a large shift in both CD8⁺ T cells and CD4⁺ Foxp3⁻ T cells from a naive CD62L⁺ CD44⁻ phenotype to a CD62L⁻ CD44⁺ Killer cell lectin-like receptor G1⁻ (KLRG1⁻) effector memory/memory precursor effector phenotype and a CD62L⁻ CD44⁺ KLRG1⁺ phenotype that could encompass both short-lived effectors and future memory cells⁴⁶ (FIG. 3H). In addition, treatment activated CD8⁺ and CD4⁺ Foxp3⁻ T cells, indicated by expression of CD69, programmed cell death protein-1 (PD-1), and the proliferation marker Ki67 (FIG. 3I). In the dLN, treatment induced an expansion of T cells and a similar bias toward CD8⁺ T cells (FIGS. 3J-3L), in addition to increased memory differentiation (FIG. 3M) and T cell activation (FIG. 3N). The full neutrophil-activating therapy did not increase the numbers of tumor T cells, although the proportion of CD8⁺ T cells increased (FIGS. 3O-3Q). Examination of T cells in the 4T1 model revealed a more blunted response, although certain commonalities were preserved, including T cell proliferation in the blood and dLN, activation and memory differentiation in the dLN, and elevated proportions of CD8⁺ T cells in the tumor.

To investigate the role of adaptive immunity in tumor clearance, we treated Rag2^{/} mice, which lack mature T cells and B cells. Neutrophil-activating therapy cleared B16 tumors in both WT and Rag2^{/} mice (FIG. 3R). Nonetheless, WT mice that had previously cleared B16 were protected from re-challenge with the same tumor, in contrast to Rag2^{/} mice or naive WT mice (FIG. 3S), indicating that neutrophil-activating therapy is capable of priming anti-tumor adaptive immune memory, even though this is not required for initial tumor clearance. These data are consistent with the low number of T cells in the tumor and the large-scale T cell activation, proliferation, and memory differentiation in the blood and dLN observed post-treatment.

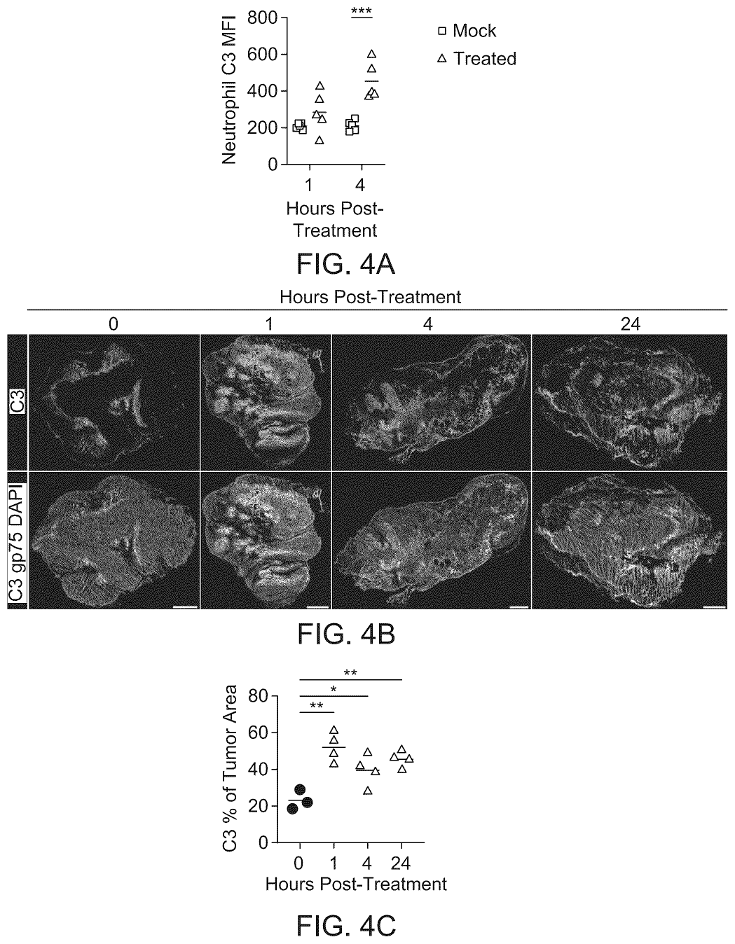

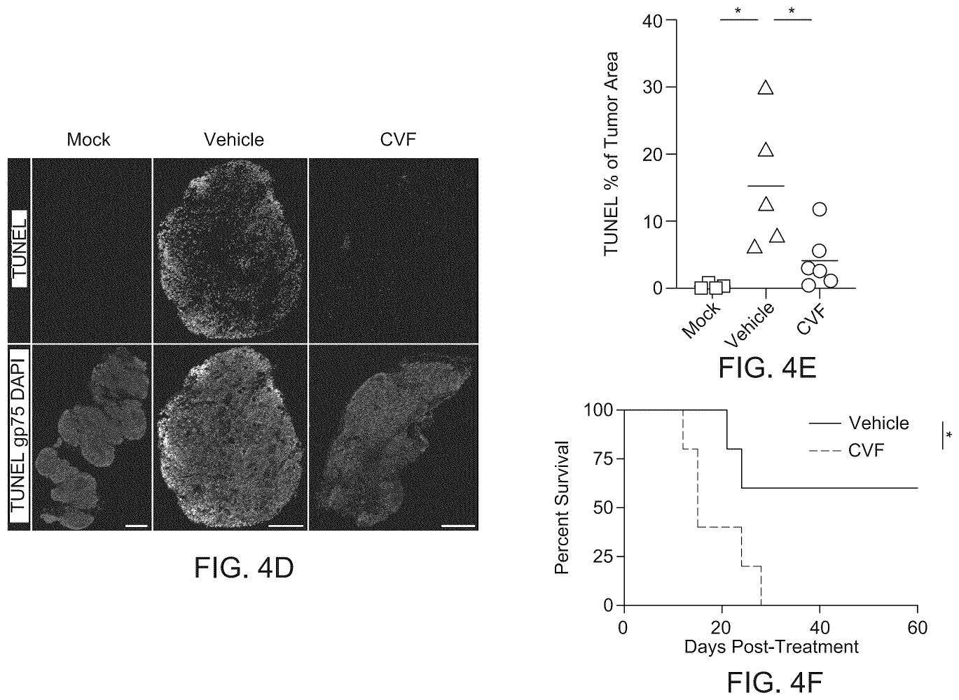

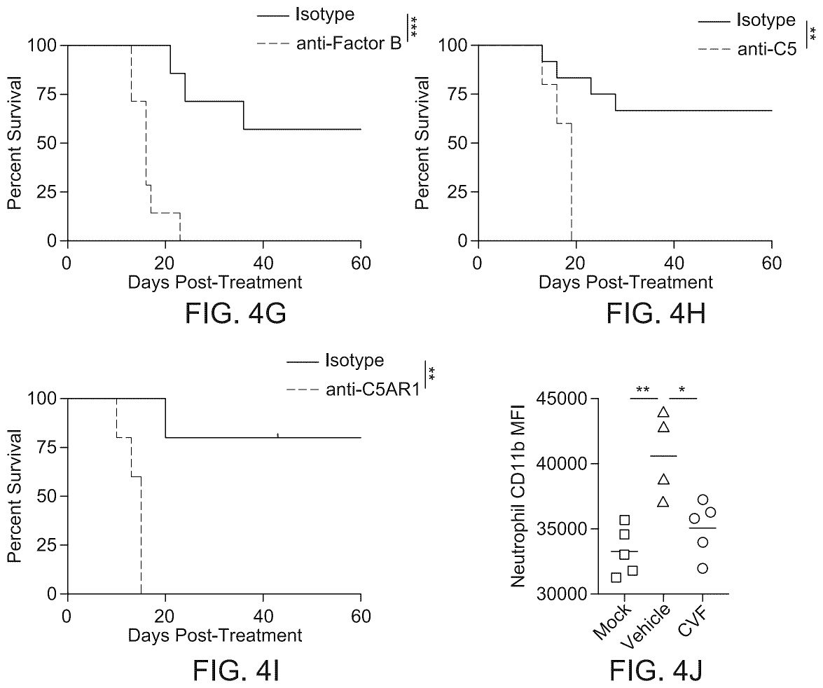

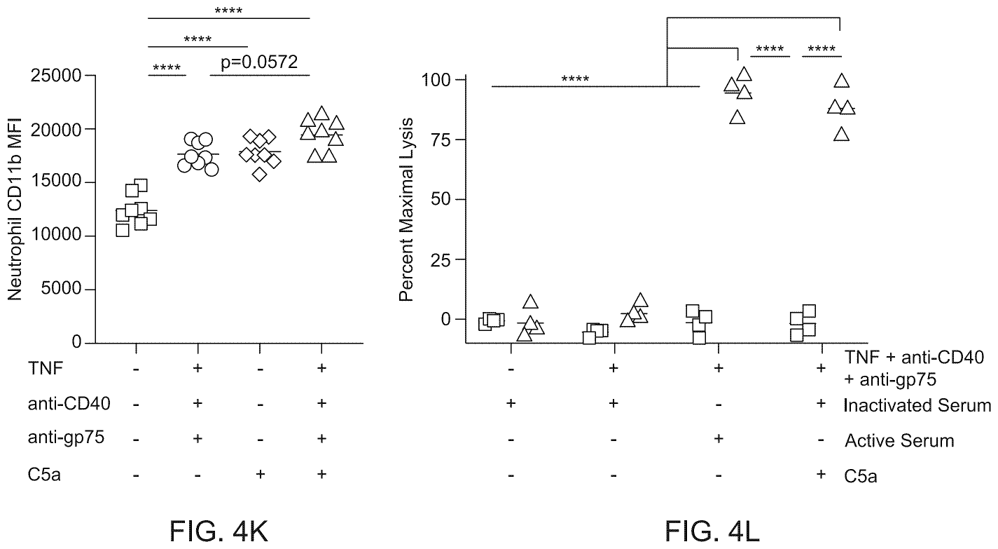

Complement activates tumor-infiltrating neutrophils through C5AR1. To identify the mechanism by which neutrophil-activating therapy stimulates neutrophils, we investigated the complement system, which generates products that are well known to stimulate neutrophil activation and recruitment.⁴⁷ Within four hours of treatment, complement component C3 was deposited on the surface of tumor-infiltrating neutrophils (FIG. 4A) and throughout the tumor (FIGS. 4B and 4C), indicating local complement activation. Complement deposition throughout the tumor was dependent on TNF signaling with potential contributions from anti-CD40 and anti-gp75. Neutrophil depletion did not alter complement deposition, suggesting that complement activation occurs upstream of neutrophil recruitment and activation.

Administration of cobra venom factor (CVF) to deplete complement prior to treatment prevented complement activation throughout tumors, which mirrored the results seen in C3 mice. CVF administration or C3 deficiency reduced tumor cell death following treatment (FIGS. 4D, 4E) and prevented tumor eradication (FIG. 4F). Depletion of Factor B, which is required for complement activation through the alternative pathway (AP), as well as depletion or deficiency of the AP positive regulator properdin, blocked tumor clearance (FIG. 4G), implicating AP complement activation as a crucial mediator of the treatment response.

The effect of complement activation was mediated through complement component C5, as C5-depleted mice failed to clear tumors (FIG. 4H). Although cleavage of C5 both generates the anaphylatoxin C5a and catalyzes the formation of the membrane attack complex (MAC) through C5b,⁴⁷ C6^{/} mice, which are incapable of forming the MAC, showed no deficit in tumor clearance. In contrast, blocking complement C5a receptor 1 (C5AR1) prevented tumor eradication (FIG. 4I), implicating C5a as the relevant complement effector. Although complement deficiency did not induce a major deficit in neutrophil recruitment, it reduced neutrophil activation in response to treatment (FIG. 4J). In contrast, complement depletion did not reduce activation of other myeloid populations in the tumor, suggesting that neutrophils are the main cell type activated by complement. Additionally, recombinant C5a activated neutrophils in vitro (FIG. 4K), and neutrophils isolated from treated tumors required C5a or serum containing functional complement to lyse tumor cells (FIG. 4L). Altogether, these data demonstrate that neutrophil-activating therapy induces complement activation through the AP, which in turn activates neutrophils through C5a-C5AR1 signaling to kill tumors.

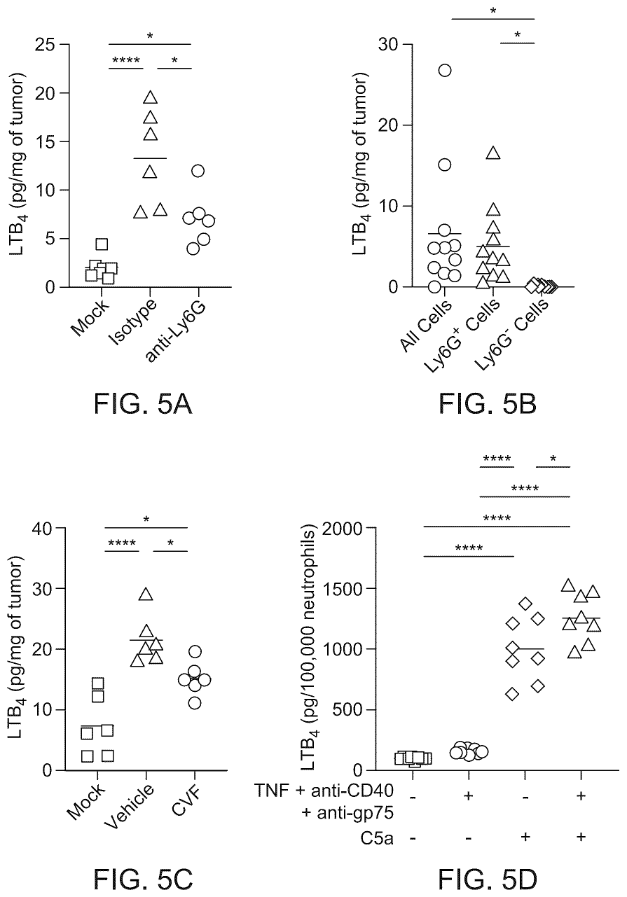

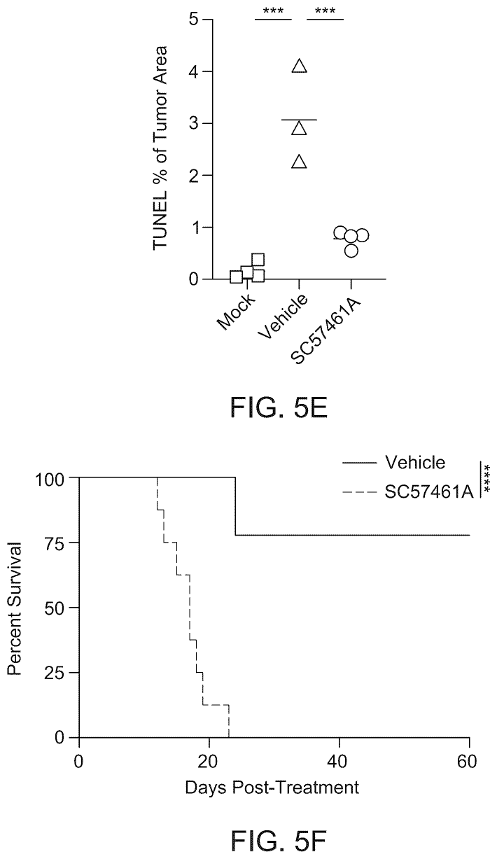

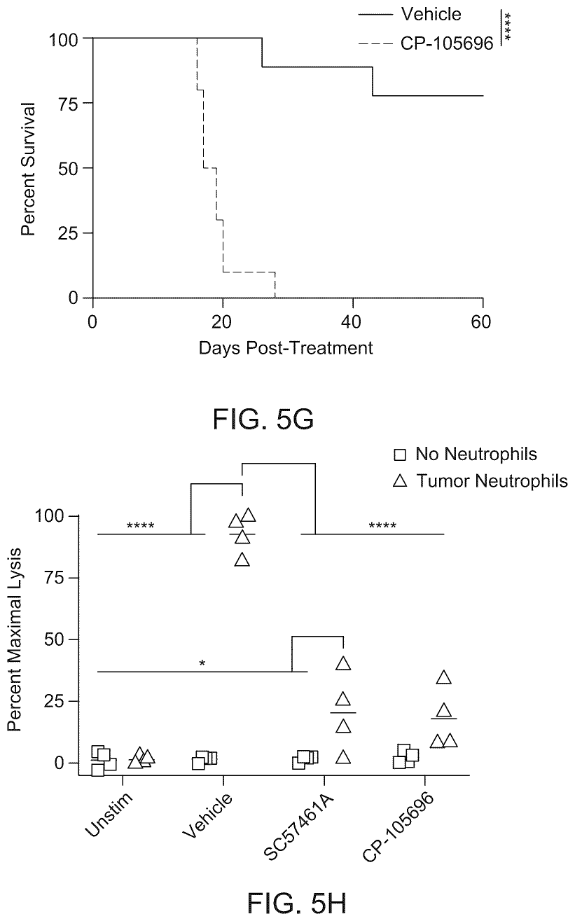

Secretion of leukotriene B4 by C5a-activated neutrophils drives tumor clearance. To identify neutrophil-derived mediators that might contribute to tumor clearance, we considered the potent pro-inflammatory lipid mediator leukotriene B4 (LTB₄).⁴⁸ Neutrophil-activating therapy induced a neutrophil-dependent increase in LTB₄ in the tumor (FIG. 5A), with neutrophils responsible for the majority of LTB₄ production (FIG. 5B). Furthermore, LTB₄ production was dependent on TNF signaling and complement (FIG. 5C), and stimulation of neutrophils with C5a in vitro induced LTB₄ (FIG. 5D). Inhibition of LTB₄ production through the leukotriene A₄ hydrolase (LTA₄H) inhibitor SC57461A prevented treatment-induced tumor cell death (FIG. 5E) and tumor clearance (FIG. 5F). The effects of LTB₄ were mediated through LTB₄ receptor 1 (BLT1), as the BLT1 antagonist CP-105696 also prevented tumor clearance (FIG. 5G), and SC57461A and CP-105696 both prevented ex vivo killing of tumor cells by neutrophils (FIG. 5H). These data demonstrate that C5a-activated neutrophils mediate tumor eradication through LTB₄ in response to neutrophil-activating therapy.

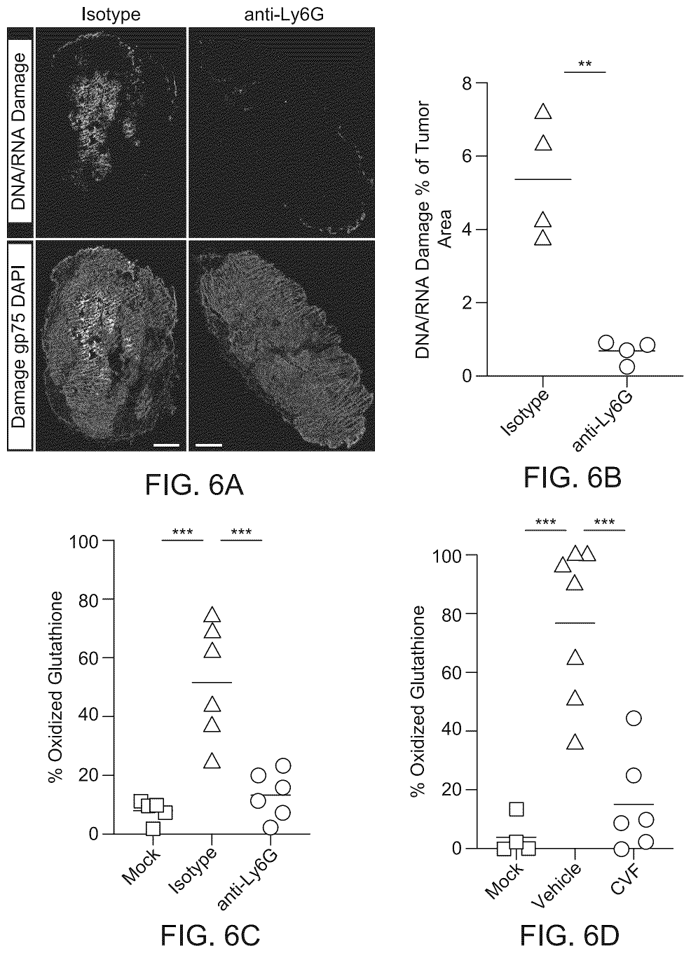

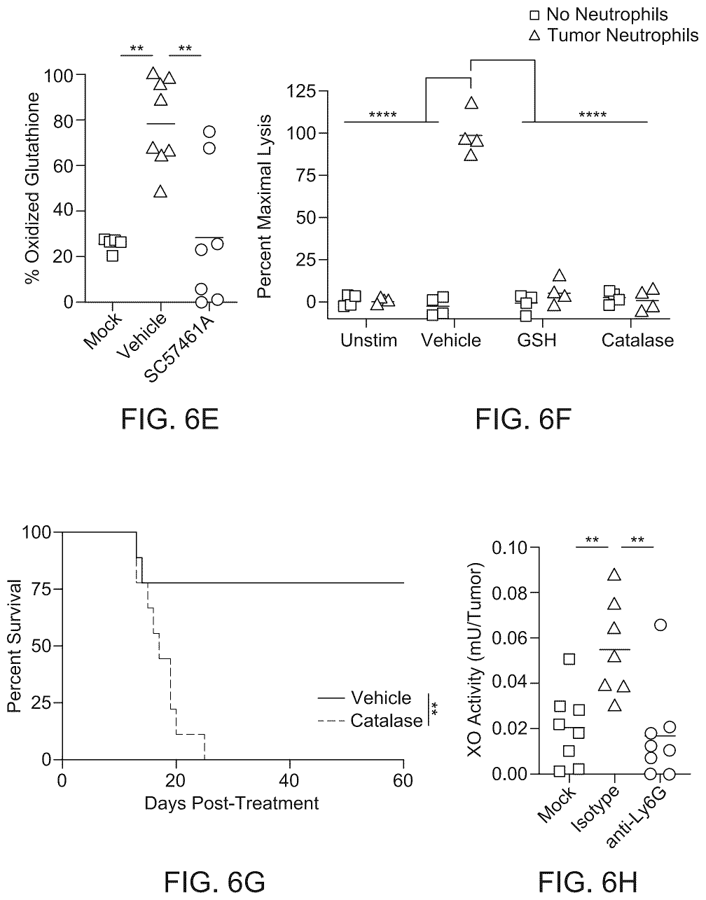

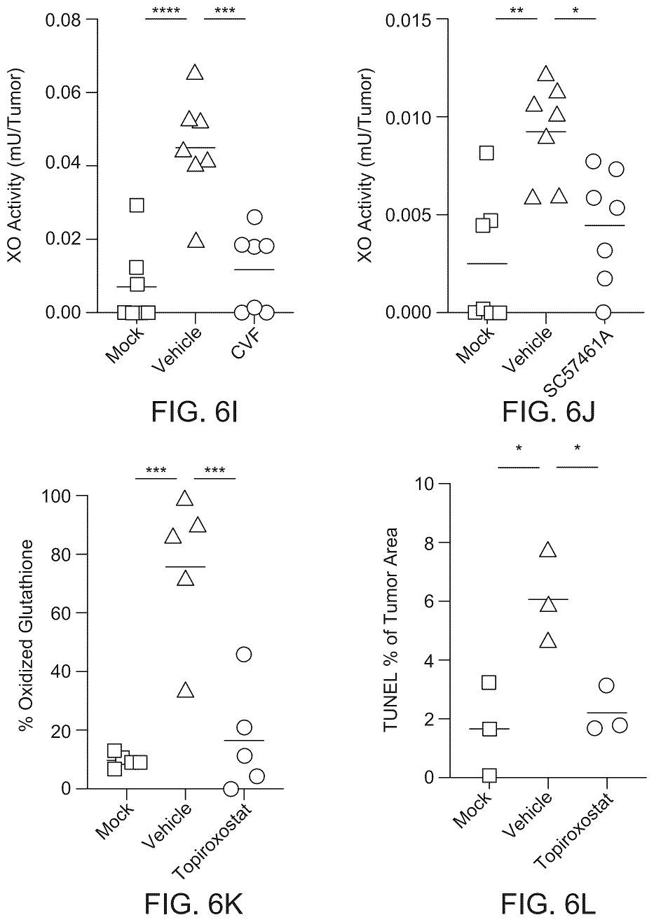

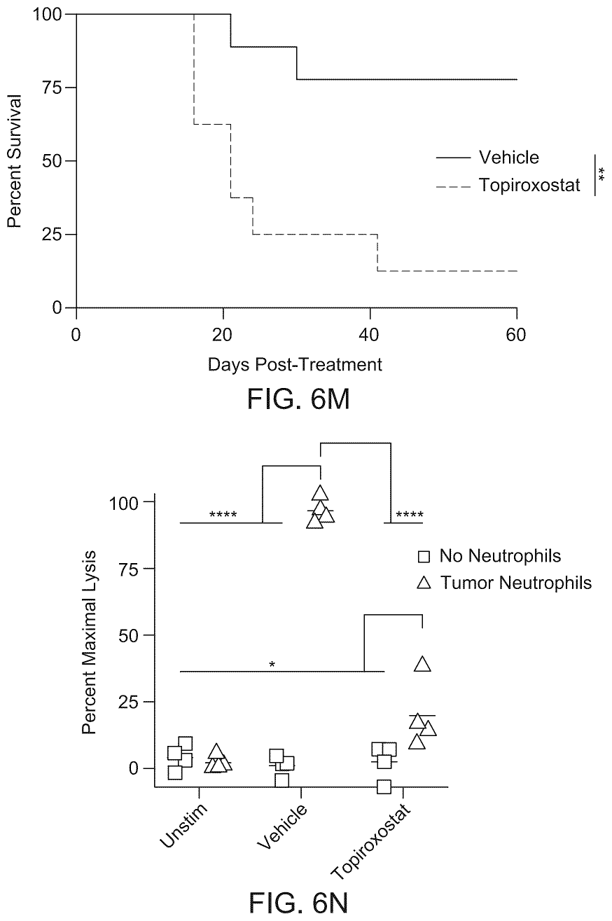

LT_B₄-dependent induction of xanthine oxidase induces oxidative damage and tumor clearance. As LTB₄⁴⁸ and C5a⁴⁹ can induce production of ROS by neutrophils, and as ROS are potent effectors of neutrophil-mediated cytotoxicity, we investigated the role of ROS in tumor clearance. Neutrophil-activating therapy induced neutrophil-dependent oxidative damage, as evidenced by oxidation of nucleic acids (FIGS. 6A and 6B) and endogenous glutathione (FIG. 6C) within the tumor. This oxidation was dependent on TNF signaling, complement, and LTB₄ (FIGS. 6D, 6E), suggesting that production of LTB₄ by C5a-activated neutrophils drives the production of ROS and resulting oxidative damage in the tumor. Scavenging of ROS with reduced glutathione (GSH) or neutralization of hydrogen peroxide with catalase blocked ex vivo killing of tumor cells by tumor-infiltrating neutrophils (FIG. 6F), demonstrating that ROS mediate neutrophil killing of tumor cells. Administration of GSH decreased tumor cell death in vivo and prevented tumor clearance following treatment. Furthermore, administration of catalase blocked tumor eradication in treated mice (FIG. 6G). These data demonstrate a critical role for ROS, and specifically hydrogen peroxide, in neutrophil-dependent tumor clearance following neutrophil-activating therapy.

Despite the requirement for neutrophils and ROS, Ncf1^{/} mice, which lack the p47 subunit of the phagocyte nicotinamide adenine dinucleotide phosphate oxidase complex (NOX) and fail to produce ROS through phagocyte NOX, were still able to clear tumors, albeit less efficiently than WT mice. Given these results, we sought an additional source of ROS in the context of this treatment. Xanthine oxidoreductase is a bidirectional enzyme capable of both xanthine dehydrogenase (XDH) and xanthine oxidase (XO) activities, and XO can produce super-oxide, serving as a source of ROS.⁵⁰ Neutrophil-activating therapy induced a neutrophil-dependent elevation in XO activity within the tumor (FIG. 6H), and depletion of complement and inhibition of LTB₄ prevented this increase in XO activity (FIGS. 6I and 6J). XO inhibition with topiroxostat did not prevent neutrophil infiltration or LTB₄ production, indicating that XO activation occurs downstream of neutrophil LTB₄ production. Importantly, inhibition of XO by topiroxostat prevented oxidation in tumors (FIG. 6K), demonstrating XO to be responsible for the ROS-mediated damage of the tumor induced by treatment. Inhibition of XO also reduced cell death (FIG. 6L), prevented tumor clearance (FIG. 6M), and inhibited ex vivo tumor cell killing (FIG. 6N). Thus, following neutrophil-activating therapy, LTB₄ production by complement-activated neutrophils induces ROS production through XO, leading to oxidative damage of tumor cells and subsequent tumor clearance.

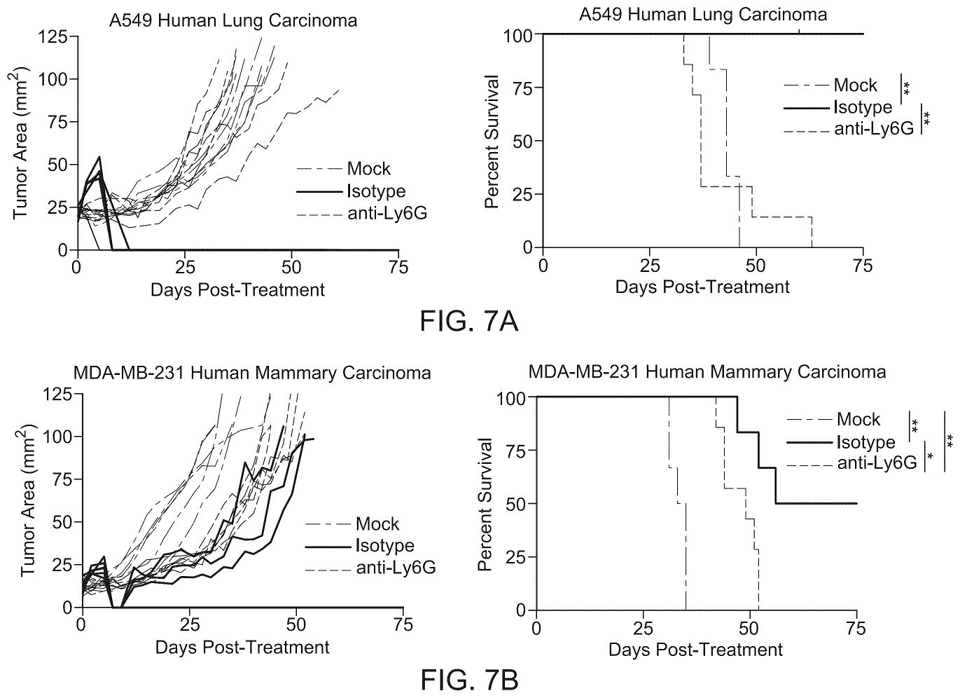

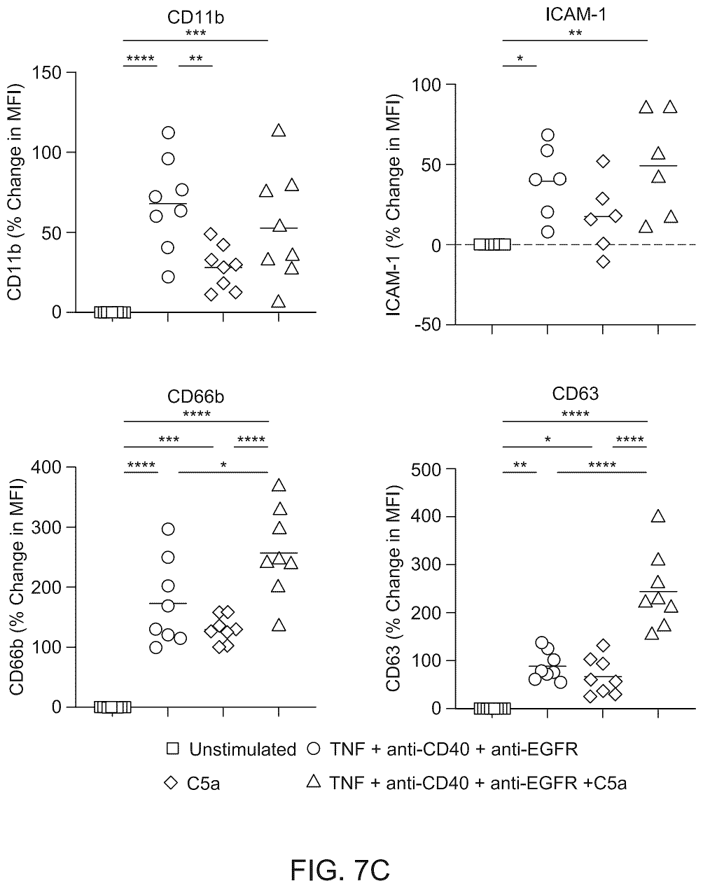

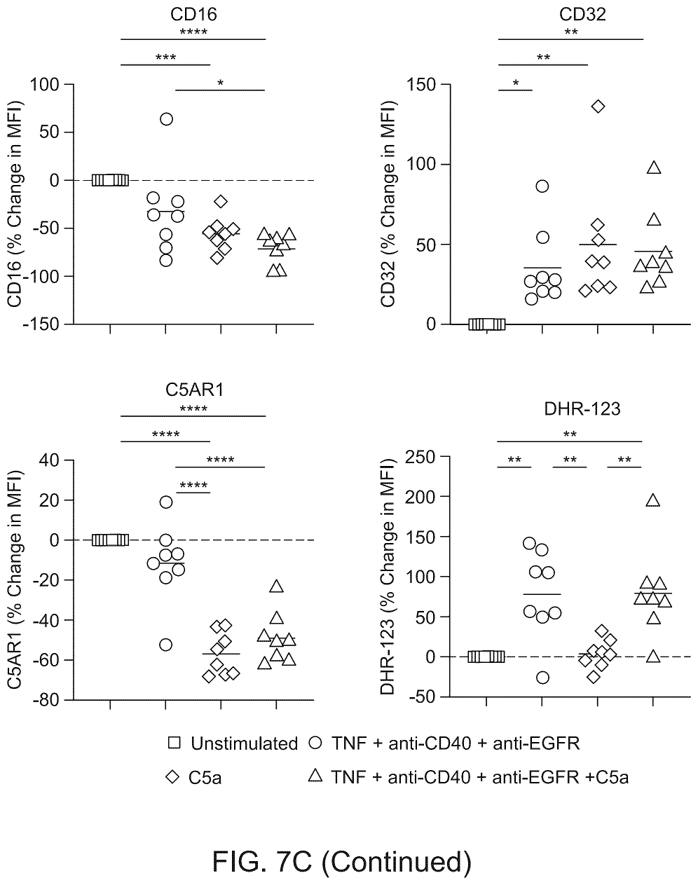

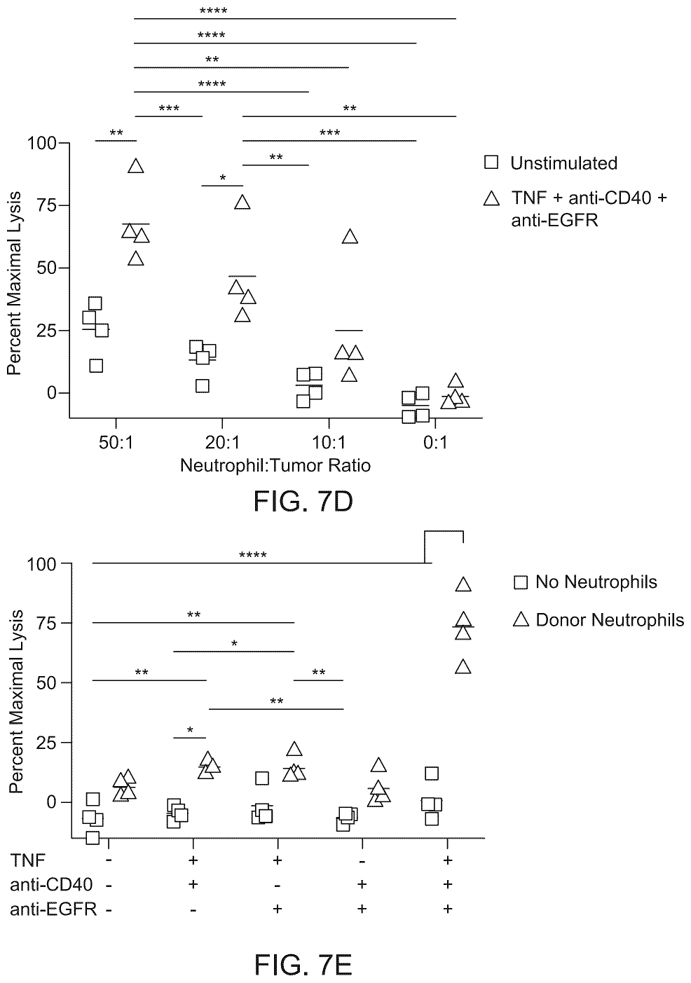

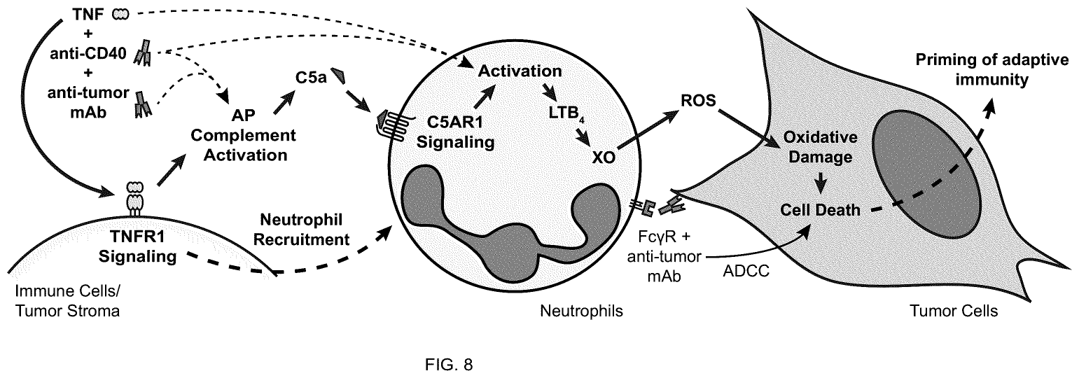

Neutrophil-activating therapy activates human neutrophils to kill tumor cells. To determine whether neutrophils could clear human tumors, we treated Rag2^{/} Il2rg^{/} mice bearing A₅₄₉ human lung carcinoma or orthotopic MDA-MB-231 human mammary carcinoma with neutrophil-activating therapy. Whereas neutrophil-sufficient mice cleared the tumors, neutrophil-depleted mice were uniformly unable to do so (FIGS. 7A and 7B). We next examined tumor cells, ultimately leading to tumor clearance. In contrast to most previous reports of neutrophil anti-tumor activity,^{1,2} this mechanism relies on activation of neutrophils rather than inhibition of their suppressive effects. Moreover, it is capable of inducing eradication of multiple tumor types in immunocompetent mice, it is effective when therapy is initiated after tumors are already established, and it can reduce metastasis. Importantly, the same combination of therapeutic components that eradicates tumors in mice activates human neutrophils to kill tumors in vitro, demonstrating that the therapy will prove effective in patients.

The identification of C5a and LTB₄ as crucial mediators of tumor clearance in this mechanism is noteworthy, as previous studies have shown that these molecules generally promote cancer. Signaling through the C5a-C5AR1 axis recruits granulocytes and MDSCs to the tumor, stimulates secretion of immunosuppressive factors by these and other tumor-resident myeloid cells, and results in inhibition of T cell responses.^{49,52-56} Similarly, LTB₄ can promote tumor growth by recruiting suppressive neutrophils and MDSCs to the tumor,⁵⁷⁻⁵⁹ and neutrophil-derived leukotrienes can support metastasis.¹⁰

However, in non-cancer contexts, C5a and LTB₄ are potent inflammatory mediators that can induce neutrophil activation and ROS-mediated tissue damage. AP activation on the neutrophil surface can produce high local concentrations of C5a, inducing neutrophil activation and extravasation into inflamed tissue.^{60,61} LTB₄ promotes neutrophil “swarming” in the tissue and vasculature, amplifying neutrophil activation, inflammation, and tissue damage.⁶²⁻⁶⁴ Elements of the mechanism we describe, such as AP-mediated complement activation, C5a-induced LTB₄ production by neutrophils, and C5a-dependent production of ROS by neutrophils and XO, can contribute to tissue damage in pathologies as diverse as inflammatory arthritis,^{60,65-67} fungal sepsis,⁶³ and acute lung injury.⁶⁸

The immunotherapeutic strategy used in this study likely taps into the neutrophil's capacity for potent cytotoxic activation that results in tissue damage in the context of these inflammatory pathologies. Although dysregulated inflammation in the tumor can induce pathological activation of neutrophils through chronic exposure to factors such as C5a and LTB₄, our work demonstrates that these inflammatory mediators have the capacity to drive tumor-eradicating neutrophil responses if applied with the appropriate threshold and context. Although inhibitors of C5AR1, LTB₄ production, and BLT1 have been evaluated as cancer treatments,^{49,59} our work here demonstrates an alternative approach by which these pathways can be exploited to direct neutrophil cytotoxic responses against the tumor.

Even though this mechanism of neutrophil-mediated tumor clearance is not dependent on adaptive immunity, it can still prime immune memory. Neutrophil-activating therapy induced activation of DC populations, and we have shown previously that treatment with tumor-binding antibody in an immunostimulatory context induces tumor antigen uptake by APCs and priming of T cells.^{69,70} We observed activation, proliferation, and memory differentiation of T cells in the blood and dLN following neutrophil-activating therapy, and mice were protected from re-challenge with the same tumor. As such, combination with T cell-targeted treatments such as immune checkpoint blockade represents an attractive avenue for future study with the potential to further enhance therapeutic efficacy. Moreover, the ability of our neutrophil-activating approach to induce an inflammatory cascade resulting in large-scale neutrophil infiltration and cytotoxicity within the tumor may help overcome barriers to the efficacy of immune checkpoint blockade, such as “cold” tumors with poor T cell infiltration.⁷¹ Additionally, although tumors could downregulate the antibody target antigen in response to therapy, as ADCC contributes to, but is not required for, tumor clearance, neutrophil-activating therapy is likely to be robust against acquired resistance that can limit the efficacy of other monoclonal antibody therapies.

This study establishes a neutrophil-activating approach to cancer therapy that can both reverse neutrophil-mediated immunosuppression and activate anti-cancer immunity. As neutrophils are numerous, plastic, and can amplify their own activation and recruitment, strategies to harness their potential to function as anti-cancer effector cells are an attractive option, and attempts to deplete or inhibit suppressive neutrophil populations may squander this powerful anti-cancer capacity. The present study defines therapeutic conditions and an in vivo mechanism by which neutrophils can be exploited to induce potent tumor eradication.

### EXPERIMENTAL MODEL AND SUBJECT DETAILS

### Animals

C57BL6/J (Jackson 000664), BALB/cJ (Jackson 000651), MMTV-PyMT (FVB/N-Tg(MMTV-PyVT)634Mu1/J, Jackson 002374), Rag2^{-/-} (B6(Cg)-Rag2^{tm1.1Cgn}/J Jackson 008449), TNFR KO (B6.129S-Tnfrsf1a^{tm1Imx} Tnfrsf1b^{tm1IJmx}/J, Jackson 003243), C3^{-/-} (B6.129S4-C3^{tm1Crr}/J, Jackson 029661), and Ncf1^{-/-} (B6N.129S2-Ncf1^{tm1Shl}/J, Jackson 027331) mice were purchased from Jackson Laboratory. Fcer1g^{-/-} mice (B6.129P2-Fcer1g^{tmRav} N12, Taconic 583) were purchased from Taconic. Rag2^{-/-} Il2rg^{-/-} mice were generated by crossing B6(Cg)-Rag2^{tm1.1Cgn}/J mice (Jackson 008449) with B6.12954-Il2rg^{tm1Wjl}/J mice (Jackson 003174). Cfp^{-/-} mice^{67,74} and C6^{-/-} mice⁷⁵ were generated as previously described. 8-12 week old female mice were used, and mice of different experimental groups and genotypes were cohoused during all experiments, with the exception of immunocompromised Rag2^{-/-} mice. Mice were randomly assigned to experimental groups. All animal studies were performed in accordance with the Stanford University Institutional Animal Care and Use Committee under protocol APLAC-17466. All mice were housed in an American Association for the Accreditation of Laboratory Animal Care-accredited animal facility and maintained in specific pathogen-free conditions.

### Cell Lines and Culture

The mouse melanoma cell line B16F10, mouse lung carcinoma line LL/2, mouse mammary carcinoma line 4T1, human lung carcinoma line A₅₄₉, and human mammary carcinoma line MDA-MB-231 were purchased from ATCC. To generate the mouse colon carcinoma line Sparkl.4640 (Syngeneic P53 APC ROSA26-LSL-eYFP Kras Lgr5-CreERT2), crypts were harvested and expanded from the colon of an adult female Lgr5-EGFP-IRES-creERT2 Trp53^{fl/fl} Apc^{fl/fl} Kras^{LSL-G12D/+} ROSA26^{LSL-eYFP/LSL-eYFP} mouse according to a published protocol.⁷⁷ Single Lgr5 stem cells build crypt-villus structures in vitro without a mesenchymal niche. Cells were dissociated into a single cell suspension and then cultured in 4-hydroxytamoxifen (Sigma). Cells were washed and allowed to form colonies in Matrigel. After 1 week in culture, roughly 50 fluorescent colonies were collected and separated from non-fluorescent colonies under stereomicroscope with fluorescence attachment (Nikon). Colonies were dissociated and plated onto tissue culture treated plates in RPMI-1640 with 10% FBS. Notably, transformed cells were then able to be passaged in the absence of supplemental growth factors or Matrigel, both of which were required for the culture of crypts prior to treatment with 4-hydroxytamoxifen.

B16, LL/2, and A₅₄₉ were cultured in DMEM (Gibco) supplemented with 10% FBS, 2 mM L-glutamine, 100 U/ml penicillin, and 100 μg/ml streptomycin (Gibco). Sparkl.4640, 4T1, and MDA-MB-231 were cultured in RPMI-1640 (Gibco) supplemented with 10% FBS, 100 U/ml penicillin, and 100 μg/ml streptomycin. Cells were tested for endotoxins using LAL Chromogenic Endotoxin Quantitation Kit (Pierce) and for mycoplasma using PlasmoTestÔ (InvivoGen), according to manufacturer's instructions.

### Human Blood

For studies involving human neutrophils, whole blood was obtained from de-identified healthy blood donors at Stanford Blood Center. Informed consent was obtained from all donors. Human neutrophils were isolated and used in assays immediately and were not maintained in culture.

### Method Details

### Tumor Implantation, Treatment, and Survival

Cell lines were harvested with trypsin-EDTA (Gibco), washed once, and injected in 50 μl phenol red-free RPMI-1640 (Gibco). B16 (2.5×10⁵ cells), Sparkl.4640 (5×10⁵ cells), and LL/2 (1×10⁵ cells) were injected subcutaneously (s.c.) into the flank of WT or KO mice on a syngeneic C57BL/6J background. 4T1 (1×10⁵ cells) was injected s.c. into the flank of WT mice on a syngeneic BALB/cJ background. A₅₄₉ (5×10⁶ cells) was injected s.c. into the flank of Rag2^{-/-} Il2rg^{-/-} mice on a C57BL6/J background. MDA-MB-231 (5×10⁶ cells) was injected orthotopically into the mammary fat pad of Rag2^{-/-} Il2rg^{-/-} mice on a C57BL6/J background. B16, Sparkl.4640, 4T1, and A₅₄₉ tumors were allowed to grow for approximately 6 days prior to treatment, LL/2 was allowed to grow for approximately 8 days prior to treatment, and MDA-MB-231 was allowed to grow for approximately 12 days prior to treatment, at which point the tumors were approximately 10-30 mm². MMTV-PyMT mice were monitored until palpable tumors developed in the breast, at approximately 8 weeks of age, and treatment was initiated when tumors reached approximately 4-10 mm².

Except where indicated otherwise, tumors were treated by intratumoral injection of tumor-binding antibody, 100 μg of agonistic anti-CD40 antibody (clone FGK4.5, Bio X Cell, Lebanon, NH), and 1 μg (ED₅₀ 0.5-3 μg/ml in L929 cytotoxicity assay) of recombinant mouse TNF (BioLegend) in 50 μl total volume in phosphate buffered saline (PBS), which is referred to in the text as neutrophil-activating therapy. As tumor-binding antibody, B16 received 100 μg anti-gp75 (clone TA99, Bio X Cell), B16-EGFP received 100 μg anti-GFP (clone F56-6A_{1.2.3}, Bio X Cell), Sparkl.4640 and LL/2 received 10 μg anti-CD44 (DS-MB-00666, RayBiotech), 4T1 and MMTV-PyMT received 100 μg anti-MHC Class I (clone 34-1-2S, Bio X Cell), and A₅₄₉ and MDA-MB-231 received 100 μg anti-human MHC Class I (clone W6/32, Bio X Cell). In all cases, tumor-binding antibodies were confirmed to bind the appropriate tumor cells by flow cytometry. Mock-treated mice received an intratumoral injection of 50 μl PBS (treatment vehicle). Treatment was administered twice, two days apart, designated as days 0 and 2 post-treatment. For MMTV-PyMT mice, treatment was repeated weekly for four cycles, so that mice were treated on days 0, 2, 7, 9, 14, 16, 21, and 23. Although tumors develop in multiple breasts in the MMTV-PyMT model, only one tumor was treated per mouse for the duration of the therapy, with the largest tumor at the time of treatment initiation chosen for treatment. For TNF dose response experiments, mice received doses of 10 ng, 50 ng, or 1 μg of TNF (BioLegend). Where indicated, tumors were injected with one or two of the three treatment components. In some experiments, tumors were injected with 1 μg recombinant mouse GM-CSF (BioLegend), 5 μg recombinant mouse IFNg (BioLegend), 1 μg recombinant mouse IL-1b (BioLegend), or 1 μg recombinant mouse IL-17A (BioLegend). Tumor areas were measured three times per week, and mice were euthanized when treated tumors exceeded 100 mm² or when tumors became ulcerated, with both indicated as a death event on the Kaplan Meier plots. Mice were censored from survival studies when they had to be euthanized for reasons unrelated to tumor progression, such as dermatitis, and were tumor-free. For re-challenge studies, C57BL/6J WT or Rag2^{-/-} mice that had cleared B16 tumors were re-challenged with 5×10⁴ B16 cells in the opposite flank 50 days after initial treatment.

### Blood Chemistry

Blood was collected from mice by retro-orbital bleed and allowed to clot for 30 minutes on ice. It was then centrifuged at 2000×g for 20 minutes at 4° C. Serum was collected from the top of the clot and centrifuged again at 2000×g for 10 minutes at 4° C. to remove residual red blood cells. Chemistry analysis was performed on the Siemens Dimension EXL200/LOCI analyzer by the Stanford University Animal Diagnostic Lab.

### Mouse In Vitro Cytotoxicity Studies

B16 tumors were treated by intratumoral injection of TNF+anti-CD40+anti-gp75 and harvested 12 hours post-treatment. Tumors were dissected away from any surrounding fat, minced, and digested in 5 mg/ml collagenase IV (Worthington) plus 0.1 mg/ml DNase I (Sigma) with continuous mixing by magnetic stir bars for 20 minutes at 37° C. in RPMI-1640 (Gibco) with 2% FBS. Following digestion, tissue was mashed through a 70 mm cell strainer (Falcon) and washed. For some studies, bone marrow was harvested by grinding bones from tumor-naive untreated mice in a mortar and pestle and mashing through a 70 mm strainer (Falcon). Neutrophils were isolated from tumor and bone marrow samples with the MojoSort Mouse Ly6G Selection Kit (BioLegend), according to the manufacturer's instructions, and used in the cytotoxicity assay.

Cytotoxicity assays were conducted using the EuTDA assay from the DELFIA TRF cytotoxicity kit (PerkinElmer) according to the manufacturer's instructions. Briefly, B16 cells were labeled for 10 minutes with BATDA, and 5×10³ labeled cells were added per well to a 96 well V-bottom plate in RPMI-1640. Neutrophils were added at a ratio of 10:1 unless otherwise stated. All co-cultures were conducted in the presence of TNF (10 ng/ml), anti-CD40 (1 μg/ml), anti-gp75 (1 μg/ml), and 10% active mouse C57BL/6 complement serum (Innovative Research), except where indicated otherwise. Mouse C57BL/6 complement serum was heat-inactivated for 40 minutes at 57° C. where inactivation is specified. In some experiments, mouse IgG2a isotype control (1 μg/ml, isotype control for anti-gp75, clone C1.18.4, Bio X Cell), anti-mouse CD16/CD32 (10 μg/ml, clone 2.4G2, Bio X Cell), recombinant mouse C5a (50 nM, R&D Systems), anti-C5a (25 μg/ml, clone 295108, R&D Systems), rat IgG2a isotype control (25 μg/ml, isotype control for anti-C5a, clone 2A₃, Bio X Cell), anti-C5AR1 (5 μg/ml, clone 20/70, BioLegend), rat IgG2b isotype control (5 μg/ml, isotype control for anti-C5AR1, clone LTF-2, Bio X Cell), SC57461A (10 μM, Cayman Chemical), CP-105696 (1 μM, Sigma), topiroxostat (10 μM, MedChem Express), or DMSO (vehicle for SC57461A, CP-105696, and topiroxostat) were added to the wells with the B16 and neutrophils. After 4 hours of co-culture at 37° C., supernatant was taken from the wells, Europium solution was added, and TDA released from lysed B16 cells was detected by TRY on a Victor X4 fluorescence microplate reader (PerkinElmer). Percent maximal lysis was determined by calculating the specific release of TDA using the formula: (experimental release−spontaneous release)/(maximum release −spontaneous release), where spontaneous release was determined by wells containing no neutrophils and maximum release was determined by wells with lysis buffer added.

For trogocytosis studies investigating transfer of tumor-binding antibody, anti-gp75 labeled with AF647 (clone TA99, Novus Biologicals) was used in place of unlabeled anti-gp75. For studies investigating transfer of B16 cell membrane, DiD′ Solid (ThermoFisher Scientific) was reconstituted in DMSO at 10 mg/ml and diluted to a working solution of 5 μg/ml in serum-free DMEM. B16 cells were labeled in this solution for 20 minutes at 37° C. at 1×10⁶ cells/ml. Labeled B16 was washed 3 times in warm media before being added to the co-culture wells with the neutrophils. Following co-culture, flow cytometry was used to identify DiD signal in neutrophils.

### Depletion and Inhibition Studies

In neutrophil depletion experiments, 500 μg anti-Ly6G (clone 1A₈, Bio X Cell) or isotype control (clone 2A₃, Bio X Cell) was administered intraperitoneally (i.p.) on days −2, 0, and 2 relative to treatment, with administration 4 hours prior to treatment on days 0 and 2. For MMTV-PyMT mice, this administration pattern was continued for four weeks for each of the treatment cycles. For neutrophil depletion studies in untreated mice, anti-Ly6G or isotype control administration began 4 days after tumor inoculation (equivalent to day −2 in treated mice), and administration continued every 2 days until the mice were euthanized. Graphing of survival for these mice began at day 6 post tumor inoculation (equivalent to day 0 in treated mice).

For TNFR blocking experiments, 100 μg of anti-TNFR1 (clone 55R-170, BioLegend), anti-TNFR2 (clone TR75-54.7, BioLegend), or isotype control (Armenian Hamster IgG, Bio X Cell) was administered i.p. once per day on days −1 through 4 relative to treatment, with administration 1 hour prior to treatment on days 0 and 2. Anti-factor B (clone 1379) was produced from a hybridoma (PTA-6230, ATCC) with serum-free CD Hybridoma Medium (Gibco) in a 1L CELLine bioreactor flask, purified with HiTrap Protein G HP columns, and buffer-exchanged to PBS in an Amicon Ultra 100 kDa centrifugal filter (Millipore). Anti-factor B or isotype control (clone MOPC-21, Bio X Cell) was administered i.p. at 1 mg once per day on days −1 through 2 relative to treatment, with administration 1 hour prior to treatment on days 0 and 2. Anti-properdin (clone 14E1) and anti-C5 (clone BB5.1) were produced as previously described⁷⁴. For properdin blocking experiments, 1 mg of anti-properdin or isotype control (clone MOPC-21, Bio X Cell) was administered i.p. on days −1 and 1 relative to treatment. For C5 blocking experiments, 800 μg of anti-C5 or isotype control (clone MOPC-21, Bio X Cell) was administered i.p. once daily on days −1 through 2 relative to treatment, with administration 1 hour prior to treatment on days 0 and 2. For C5AR1 blocking experiments, anti-C5AR1 (clone 20/70, BioLegend) and isotype control (clone LTF-2, Bio X Cell) were deglycosylated with the deGlycIT kit (Genovis) prior to administration in order to abrogate Fc receptor binding and prevent depletion of anti-C5AR1-bound neutrophils,⁷⁸ and 100 μg of anti-C5AR1 or isotype control were administered daily on days −1 through 3 relative to treatment, with administration 1 hour prior to treatment on days 0 and 2.

Reduced L-glutathione (Sigma) was dissolved in PBS and administered i.p. at 500 mg/kg at hours −1, 0, 2, 4, and 8 relative to treatment on days 0 and 2, as well as twice per day on days 1 and 3. Catalase (C40, Sigma) was dissolved in PBS and administered i.p. at 500 mg/kg twice per day on days 0, 1, 2, and 3 relative to treatment, with administrations on days 0 and 2 coming immediately prior and 4 hours after treatment. CVF (from Naja naja kaouthia, Millipore), which depletes complement components C3 and C5 from blood through fluid-phase activation,⁷⁹ was diluted in PBS and administered at 50 μg i.p. daily on days −2 through 2 relative to treatment, with administration 30 minutes prior to treatment on days 0 and 2. SC57461A (Cayman Chemical, Ann Arbor, MI) was dissolved in DMSO at 150 mg/ml, diluted in PBS, and administered i.p. at 75 mg/kg twice per day on days −1 through 3 relative to treatment, with administration 1 hour prior and 4 hours after treatment on days 0 and 2. CP-105696 (Sigma) was dissolved in DMSO at 400 mg/ml, diluted in 25% 2-hydroxypropyl-b-cyclodextrin (Cayman Chemical), and administered i.p. at 100 mg/kg twice per day on days 0 through 3 relative to treatment, with administration 1 hour prior and 4 hours after treatment on days 0 and 2. Topiroxostat (MedChemExpress) was dissolved in 0.2N NaOH in PBS, the pH was adjusted with HCl, and it was administered i.p. at 150 mg/kg on days 0 and 2, 3 hours prior to treatment.

### Metastasis Studies

For B16 experimental metastasis studies, mice were implanted with 2.5×10⁵ B16 cells expressing tdTomato s.c. in the flank. Seven days after implantation, 2×10⁵ B16 cells expressing tdTomato were injected i.v. by the tail vein. Ten hours after tail vein injection, the s.c. tumors were treated with PBS or TNF+anti-CD40+anti-gp75, and the primary tumors were treated again two days later according to the standard treatment protocol. Nine days after tail vein injection, the mice were euthanized, the lungs harvested, and fluorescence images were acquired under a stereomicroscope with fluorescence attachment (Nikon). Discrete fluorescent metastases visible on the exterior of the lungs were counted to obtain metastasis counts. The average nodule area for metastases was determined using ImageJ by thresholding the image to remove background, using the analyze particles function to obtain the total area of the metastases, and dividing this area by the number of metastases.

For 4T1 metastasis studies, 1×10⁵ 4T1 tumor cells were implanted orthotopically in the mammary fat pad. One week post-implantation, when the primary tumor had reached a size of 16-30 mm², the primary tumor was treated by intratumoral injection of neutrophil-activating therapy or PBS mock treatment, with two injections two days apart, according to the standard protocol. The mice were euthanized 30 days post-implantation and pulmonary metastases were enumerated as previously described⁸⁰ by intra-tracheal injection of India ink (15% India Ink, 85% PBS, 0.1% NaOH). India ink-injected lungs were washed in 3 mL Fekete's solution (50% ethanol, 6% formaldehyde, and 3% glacial acetic acid) and then placed 5 mL fresh Fekete's solution overnight. White tumor nodules against a black lung background were counted manually.

### Knockout of TNFR1 in B16 Using CRISPR-Cas9

pSpCas9(BB)-2A-GFP (PX458) was a gift from Feng Zhang (Addgene plasmid #48138; http://n2t.net/addgene:48138; RRID: Addgene_48138).⁷⁶ Two sgRNA target sequences for mouse Tnfrsf1a were chosen from the Brie library⁸¹: AGACCTAG CAAGATAACCAG and GATGGGGATACATCCATCAG, referred to in the text as Tnfrsf1a sgRNA1 and Tnfrsf1a sgRNA2, respectively. An sgRNA targeting the irrelevant E. coli b-galactosidase gene (LacZ) was also included as a control. Oligos for these sgRNA target sequences were synthesized by the Stanford Protein and Nucleic Acid facility, with end overhangs to enable cloning into the BbsI site of the PX458 backbone. Oligos were phosphorylated with T4 PNK (NEB) and annealed in a thermocycler at 37° C. for 30 minutes, followed by 95° C. for 5 minutes, and ramping down to 25° C. at 5° C./minute. PX458 was digested with BbsI (NEB) and gel purified using the QIAquick Gel Extraction Kit (Qiagen). Phosphorylated and annealed oligos were ligated into BbsI-cut PX458 with T4 DNA ligase (NEB) and transformed into Stellar Electrocompetent Cells (Clontech) by electroporation with the GenePulser Xcell (BioRad). Plasmids were prepared with the Plasmid Plus Maxi Kit (Qiagen) and transfected into B16 cells using Lipofectamine 2000 (Thermo Fisher Scientific). Successfully transfected cells positive for expression of GFP were sorted on the FACSAria II (BD), followed by three successive rounds of sorting for cells negative for both GFP and TNFR1 staining using APC anti-TNFR1 (clone 55R-286, BioLegend), to achieve a population of cells lacking expression of TNFR1 with the transient expression of GFP and CRISPR machinery removed.

### B16-EGFP

To generate B16 cells expressing EGFP on their surface, DNA encoding a fusion of mouse Igk signal peptide, EGFP, and the transmembrane domain of mouse PDGFR was synthesized using GeneArt Gene Synthesis (Invitrogen). The synthesized gene was digested with BamHI and EcoRI (NEB) and gel purified using the QIAquick Gel Extraction Kit (Qiagen). This was ligated into pLVX-EF 1a-IRES-Puro (Clontech) with T4 DNA ligase (NEB) and transformed into Stellar Electrocompetent Cells (Clontech) by electroporation with the GenePulser Xcell (BioRad). Plasmids were prepared with the Plasmid Plus Maxi Kit (Qiagen) and transfected into 293T cells together with the psPax2 and pCMV-VSV-G plasmids using Lipofectamine 2000 (Thermo Fisher Scientific). Virus was collected and used to transduce B16 cells, and successful transductants were selected using 1 μg/ml puromycin (ThermoFisher Scientific).

### Flow Cytometry

Tumors were digested with collagenase IV and DNase I as described above. Following digestion, tissue was mashed through a 70 mm cell strainer (Falcon), washed, red blood cells were lysed with ACK buffer for 1 minute, and cells were washed again prior to antibody staining. Blood was harvested into PBS plus 20 mM EDTA by cardiac puncture or retro-orbital bleed, lysed in ACK buffer for 5 minutes, and washed prior to antibody staining. Bone marrow was harvested by grinding the femur and tibia in a mortar and pestle and mashing through a 70 mm strainer (Falcon), or by flushing femurs with a syringe and needle, then lysed in ACK buffer for 5 minutes and washed prior to antibody staining. Spleens were harvested by mashing through a 70 mm cell strainer (Falcon), then lysed in ACK buffer for 5 minutes and washed prior to antibody staining.

Live cells were stained with antibodies on ice for 20 minutes in FACS buffer (HBSS 1% BSA 5 mM EDTA) with Brilliant Stain Buffer Plus (BD). Following staining, cells were washed twice in FACS buffer and resuspended in 1 μg/ml DAPI plus AccuCount Fluorescent Particles (Spherotech) for absolute count determination. In some experiments, live cells were stained with Live/Dead Fixable Blue Stain (Invitrogen) in HBSS on ice for 20 minutes prior to antibody staining, and DAPI was not used. Cells for the HSC/progenitor experiment were viability stained with 800CW NHS Ester (Li-Cor #929-70020) at a 1:4000 dilution of a 1 mg/ml DMSO stock, for 20 minutes in PBS on ice prior to antibody staining. For assessment of neutrophil depletion efficiency and experiments analyzing Foxp3 and Ki67, cells were stained with Live/Dead Fixable Blue Stain (Invitrogen) in HBSS on ice for 20 minutes, cells were fixed and permeabilized prior to antibody staining using the Foxp3/Transcription Factor Staining Kit (eBioscience) or the True-Nuclear Transcription Factor Set (BioLegend) according to the manufacturer's instructions, and DAPI was not used. This staining following fixation/permeabilization allows identification of neutrophils with depleting antibody-bound extracellular Ly6G by staining intracellular Ly6G. Samples were acquired on an LSRFortessa (BD), except for the HSC/progenitor data, which was acquired on a Cytek Aurora using SpectroFlo V2.2.0.3. The following antibodies were used: BUV395 anti-CD45 (clone 30-F11, BD), BV421 anti-ICAM-1 (clone YN1/1.7.4, BioLegend), BV480 anti-MHC II (clone M5/114.15.2, BD), BV650 anti-CD11b (clone M1/70, BioLegend), PE-Cy7 or BUV737 anti-Ly6G (clone 1A₈, BD/BioLegend), PE or AF647 anti-C3 (clone 11H9, Novus Biologicals), APC-R700 anti-CD11c (clone N418, BD), APC-Cy7 or BV785 anti-Ly6C (clone HK1.4, BioLegend), CD177 AF647 (clone Y127, BD Biosciences), SIRPa FITC (clone P84, BioLegend), Siglec F BV480 (clone E50-2440, BD Biosciences), CD101 PE-Cy7 (clone Moushi101, ThermoFisher Scientific), CD14 APC-Cy7 (clone Sa14-2, BioLegend), PD-L1 BV421 or BV711 or PE-Cy7 (clone 10F.9G2, BioLegend), XCR1 APC (clone ZET, BioLegend), F4/80 APC-Cy7 (clone BM8, BioLegend), CD206 FITC (clone C068C2, BioLegend), CD80 PerCP-eF710 (clone 16-10A₁, ThermoFisher Scientific), CD86 BUV737 (clone GL1), B220 BV711 (clone RA3-6B2, BioLegend), TCRb BV421 (clone H57-597, BioLegend), NK1.1 PE-Cy7 (clone PK136, BioLegend), CD8a BV510 (clone 53-6.7, BioLegend), CD4 BV711 (clone RM4-5, BioLegend), Foxp3 AF488 (clone MF23, BD Biosciences), CD25 BV650 (clone PC61, BioLegend), CD62L PerCP-Cy5.5 (clone MEL-14, BioLegend), CD44 APC-eF780 (clone IM7, ThermoFisher Scientific), KLRG1 PE (clone 2F1/KLRG1, BioLegend), PD-1 BV785 (clone 29F.1A₁₂, BioLegend), CD69 APC (clone H1.2F3, BD Biosciences), Ki67 AF700 (clone SolA15, ThermoFisher Scientific), CD34 eF450 or AF700 (clone RAM34, ThermoFisher Scientific), IL-7Ra AF700 (clone A₇R34, ThermoFisher Scientific), Sca-1 BV711 (clone D7, BioLegend), Sca-1 PE-Cy7 (clone E13-161/7, BioLegend), cKit FITC (clone 2B8, ThermoFisher Scientific), CD16/32 PE (clone S17011E, BioLegend), CD3F PE-Cy7 (clone 17A₂, ThermoFisher Scientific), CD4 PE-Cy7 (clone GK1.5, ThermoFisher Scientific), CD8a PE-Cy7 (clone 53-6.7, ThermoFisher Scientific), CD11b PE-Cy7 (clone M1/70, ThermoFisher Scientific), B220 PE-Cy7 (clone RA3-6B2, ThermoFisher Scientific), NK1.1 PE-Cy7 (clone PK136, ThermoFisher Scientific), Flt3 APC (clone A₂F10, BioLegend), CD106 Pacific Blue (clone 429 (MVCAM.A), BioLegend), CD115 BV605 (clone AFS98, BioLegend), Ly6G PE-Cy5 (clone 1A₈, ThermoFisher Scientific), CD81 PerCP-Cy5.5 (clone Eat-2, BioLegend), H-2K^{b} PE (clone AF6-88.5, BD Biosciences), H-2D^{b} APC (clone KH95, BioLegend), fluorescein anti-gp75 (clone TA99, Bio X Cell, labeled using NHS-Fluorescein (Thermo Scientific)), MPO FITC (clone 2D4, Abcam), citrullinated Histone H3 (Abcam ab5103), donkey anti-rabbit IgG PE (BioLegend Poly4064), and unconjugated anti-CD16/CD32 (clone 2.4G2, Bio X Cell) to block Fc receptors. For dihydrorhodamine-123 (DHR-123) staining of tumor samples, 5 mM DHR-123 in DMSO (Invitrogen) was added to the collagenase mixture at a dilution of 1:4000 and allowed to stain for the duration of the 20-minute collagenase digestion. Flow cytometry data was analyzed using FlowJo software (BD). Staining levels were quantified using the median fluorescence intensity (MFI).

### May-Gru€Nwald-Giemsa Staining

Tumors were harvested, processed, and stained for flow cytometry as described above, 24 hours after treatment or mock treatment. Cells were stained with FITC anti-CD45 (clone 30-F11, BioLegend), APC-Cy7 anti-CD11b (clone M1/70, BioLegend), and PE anti-Ly6G (clone 1A₈, BioLegend), and CD45⁺ CD11b⁺Ly6G⁺ neutrophils were sorted on a FACSAria II (BD). Sorted neutrophils were resuspended at 5×10⁵ cells/ml in FACS buffer, and 200 μl was spun onto a slide using the StatSpin CytoFuge 2 at 850 rpm for 10 minutes. Slides were dried and then stained for 4 minutes in May-Gru€nwald stain solution (Electron Microscopy), transferred directly into 4% Giemsa stain solution (Electron Microscopy) for 4 minutes, and washed twice with water for 30 seconds each. Slides were dried and coverslips were mounted using Cytoseal 60 (Richard Allen Scientific). Stained cells were imaged on a Keyence BZ-X810 microscope (Keyence) with the 20× objective and a resolution of 1920×1440 pixels.

### Immunofluorescence

Tumors were dissected away from surrounding fat, fixed in 2% paraformaldehyde for 2 hours at 4° C., equilibrated in a 30% sucrose solution at 4° C., and embedded and frozen in O.C.T. Compound (Tissue-Tek). Slides were cut to 6 mm and blocked with 1% BSA and 10% serum matched to the secondary antibody species. The following antibodies were used for immunofluorescence: PE anti-Ly6G (clone 1A₈, BioLegend), DyLight 650 anti-gp75 (clone TA99, Bio X Cell, labeled using DyLight 650 NHS Ester (Thermo Scientific)), fluorescein anti-gp75 (clone TA99, Bio X Cell, labeled using NHS-Fluorescein (Thermo Scientific)), FITC anti-DNA/RNA damage (clone 15A₃, recognizing 8-hydroxy-2′-deoxyguanosine/8-oxo-7,8-dihydroguanine/8-oxo-7,8-dihydroguanosine, Abcam), and AF647 anti-C3 (clone 11H9, Novus Biologicals). DAPI (Invitrogen) was stained at 1 μg/ml. FITC/fluorescein signal was amplified using AF488 anti-FITC (ThermoFisher Scientific), and PE was amplified using biotin anti-PE (clone PE001, BioLegend) followed by DyLight 594 streptavidin (BioLegend) or DyLight 649 streptavidin (BioLegend). Prior to use of biotinylated antibodies, endogenous biotin was blocked using the Avidin/Biotin Blocking Kit (Vector Laboratories). Prior to DNA/RNA damage staining, sections were permeabilized with 0.1% Triton X-100 in 0.1% sodium citrate. TUNEL (Terminal deoxynucleotidyl transferase dUTP nick end labeling) staining was performed using the In Situ Cell Death Detection Kit, TMR Red (Roche) according to the manufacturer's instructions. Briefly, tissue sections were fixed with 4% paraformaldehyde for 20 minutes on ice prior to treatment with 0.1% Triton X-100 in 0.1% sodium citrate for permeabilization. Sections were washed in PBS before incubation for 60 minutes at 37° C. with antibodies and TdT enzyme, followed by washing. Images were acquired by tile scanning using a Zeiss LSM 700 confocal laser scanning microscope (Carl Zeiss Microscopy) or a Keyence BZ-X810 microscope (Keyence) using the 20× objective and a resolution of 960×720 pixels per tile. Tumor immunostainings were repeated independently at least 2 times in at least biological triplicate and whole tissue section images were acquired. Stitching of images acquired with the LSM 700 was performed using ZEN software, and stitching of images acquired with the BZ-X810 was performed using BZ-X800 Analyzer software. Multiple tumors from the same experiment were embedded together in the same block, stained together in the same section, and acquired together in a tile scan across the entire section. Individual tumors were then cropped from the full tile scan image for display in figures. Images were overlaid and color channel levels were adjusted in Photoshop (Adobe), with individual color channels receiving individual level adjustments based on the staining intensity. All parameters that were quantified were acquired with identical microscope settings and adjusted identically in Photoshop, and all adjustments were applied equally across the entire tumor. Quantification was performed in ImageJ, using the wand tool on the overlaid multichannel image to draw a border around the tumor and then measuring the percent area within that border with signal for the channel of the marker quantified.

LTB₄ ELISA. Tumors were dissected away from surrounding fat 24 hours post-treatment and lysed in PBS using 3 mm zirconium beads (Benchmark Scientific) in the BeadBug Microtube Homogenizer (Benchmark Scientific) for 2 cycles of 45 seconds at 3000 rpm. Crude lysate was centrifuged for 15 minutes at 16000×g at 4° C. to obtain clarified lysate. Clarified lysate was deproteinized by ethanol precipitation by adding 4 volumes of 100% ethanol, incubating on ice for 5 minutes, and centrifuging for 10 minutes at 3000×g at 4° C. Deproteinized supernatant was transferred to a new tube, ethanol was removed by evaporation at room temperature, and samples were brought to the appropriate volume in ELISA assay buffer. LTB₄ content was determined with the Leukotriene B4 ELISA kit (Cayman Chemical 520111) according to the manufacturer's instructions and read with a Victor X4 fluorescence microplate reader (PerkinElmer).

Ex vivo neutrophil LTB₄ assay. B16 tumors were harvested from mice 12 hours after treatment with TNF+anti-CD40+anti-gp75 and digested with collagenase IV and DNase I as described above. Tumor samples were then split into two halves, with one half undergoing selection with the MojoSort Mouse Ly6G Selection Kit (BioLegend) to generate Ly6G+ and Ly6G-depleted tumor samples and the other half undergoing depletion with an isotype control antibody and streptavidin nanobeads (BioLegend) to generate the “all cells” condition. These selected samples were then plated in 200 μl of Opti-MEM (Gibco) and incubated for 30 minutes at 37° C. Following the incubation, the supernatant was collected, centrifuged to remove cells and debris, and analyzed for LTB₄ using the Leukotriene B4 ELISA kit (Cayman Chemical 520111) as described above.

Mouse in vitro neutrophil stimulations. Bone marrow was harvested by grinding bones in a mortar and pestle and mashing through a 70 mm strainer (Falcon). Neutrophils were isolated by negative selection with the MojoSort Mouse Neutrophil Isolation Kit (BioLegend). Following isolation, neutrophils were plated in Opti-MEM (Gibco) at 1×10⁵ cells in 100 μl and stimulated with 10 ng/ml TNF, 1 μg/ml anti-CD40, 1 μg/ml anti-gp75, and/or 50 nM recombinant mouse C5a (R&D Systems) for 30 minutes at 37° C. After 30 minutes, the supernatant was collected, centrifuged to remove cells and debris, and analyzed for LTB₄ using the Leukotriene B4 ELISA kit (Cayman Chemical 520111) as described above. Additionally, stimulated neutrophils were stained for activation markers and analyzed by flow cytometry as described above.

Determination of oxidized glutathione content. Tumors were dissected away from surrounding fat 24 hours post-treatment and lysed in mammalian lysis buffer (Abcam ab179835) using 3 mm zirconium beads (Benchmark Scientific) in the BeadBug Microtube Homogenizer (Benchmark Scientific) for 2 cycles of 45 seconds at 3000 rpm. Crude lysate was centrifuged for 15 min at 16000×g at 4° C., and clarified lysate was deproteinized using the Deproteinizing Sample Preparation Kit—TCA (Abcam ab204708) according to the manufacturer's instructions. Glutathione was detected using the GSH/GSSG Ratio Detection Assay Kit II (Abcam ab205811) according to the manufacturer's instructions, reading the resulting signal with a Victor X4 fluorescence microplate reader. The percentage of oxidized glutathione was calculated from the reduced glutathione and total glutathione values determined by the kit.

### XO Assay

Tumors were dissected away from surrounding fat 24 hours post-treatment and lysed in XO assay buffer (Abcam) using 3 mm zirconium beads (Benchmark Scientific) in the BeadBug Microtube Homogenizer (Benchmark Scientific) for 2 cycles of 45 seconds at 3000 rpm. Crude lysate was centrifuged for 10 minutes at 16000×g at 4° C. to obtain clarified lysate. XO activity of the lysate was determined using the Xanthine Oxidase Activity Assay kit (Abcam), setting up the fluorometric assay and performing calculations according to the manufacturer's instructions, and reading fluorescence with a Victor X4 fluorescence microplate reader.

ROS assays. For the luminol assay, B16-bearing mice were anesthetized 4 hours post-treatment, and 50 μl luminol sodium salt (Sigma-Aldrich) at 20 mg/mL in PBS was administered intratumorally. Mice were immediately imaged using the IVIS Lumina system (Xenogen). Signal intensity was quantified as photons/second (p/s) over a one-minute exposure in equally sized regions of interest placed over the tumor, using Living Image software (Caliper Life Sciences).

For the OxyBurst assay, neutrophils were isolated from naive bone marrow or treated tumors, and 1×10⁵ neutrophils were plated in a 96-well plate together with 5×10³ B16 tumor cells. The cells were stimulated as in the cytotoxicity studies and cultured for 4 hours at 37° C. in the presence of 10 μg/ml OxyBurst Green H2HFF BSA. After co-culture, the plate was read on a Victor X4 fluorescence microplate reader (PerkinElmer) with 485 nm excitation and 535 nm emission.

Human neutrophil studies. Human neutrophils were isolated from whole blood obtained from de-identified blood donors using the EasySep Direct Human Neutrophil Isolation Kit (StemCell Technologies) according to the manufacturer's instructions. For activation marker studies, neutrophils were plated at 1×10⁶ cells/ml in Opti-MEM (Gibco) and stimulated for 30 minutes at 37° C. with human TNF (50 ng/ml, BioLegend), anti-human CD40 (1 μg/ml, clone G28.5, Bio X Cell), anti-human EGFR (1 μg/ml, Cetuximab biosimilar, Bio X Cell), or human C5a (50 nM, R&D Systems), as indicated in the figures. In some experiments, 5 mM DHR-123 in DMSO (Invitrogen) was added to the stimulation well at a dilution of 1:4000. Following stimulation, the cells were stained with antibodies on ice for 20 minutes in FACS buffer (HBSS 1% BSA 5 mM EDTA) with Brilliant Stain Buffer Plus (BD). Following staining, cells were washed twice in FACS buffer and resuspended in 1 μg/ml DAPI. Samples were acquired on an LSRFortessa (BD). The following antibodies were used: APC-Fire 750 anti-CD11b (clone M1/70, BioLegend), ICAM-1 FITC (clone HA58, BioLegend), CD16 BV711 (BioLegend, clone 3G8), CD32 BV786 (clone FLI8.26, BD Biosciences), CD66b AF647 (clone G10F5, BioLegend), CD63 BV510 (clone H5C6, BD Biosciences), and anti-C5AR1 PE (clone S5/1, BioLegend). Cytotoxicity assays were performed with the EuTDA assay from the DELFIA TRF cytotoxicity kit (PerkinElmer) according to the manufacturer's instructions. A₅₄₉ cells were labeled for 30 minutes with BATDA, and 1×10⁴ labeled cells were added per well to a 96 well V-bottom plate in RPMI-1640. Neutrophils were added at a ratio of 50:1 unless specified otherwise. All co-cultures were conducted in the presence of TNF (10 ng/ml), anti-CD40 (1 μg/ml), anti-gp75 (1 μg/ml), and 10% pooled human complement serum (Innovative Research) for 1 hour, except where indicated otherwise.

### Quantification and Statistical Analysis

Statistics. Statistical tests were performed in Prism (GraphPad Software, Inc.). Statistical tests used are listed in the figure legends. In cases where conditions were compared across multiple time points or cell types, statistical significance was determined by two-way ANOVA with Tukey's multiple comparisons test, comparing only the conditions within each time point or cell type. For in vitro cytotoxicity studies with multiple conditions and groups, statistical significance was determined by two-way ANOVA with Tukey's multiple comparisons test, comparing all conditions and groups with all other conditions and groups. Plots display individual biological replicates obtained from distinct mice, with a line at the mean. For all figures, * denotes p<0.05, ** denotes p<0.01, *** denotes p<0.001, **** denotes p<0.0001, and n.s. indicates not significant, with the exception of Kaplan Meier plots with multiple comparisons, in which case the asterisks are assigned based on Bonferroni-corrected p values. For all experiments, n represents the number of mice or the number of samples. Exact n values and the number of independent experiments are provided in the figure legends.

### Example 2. Neutrophil-Activating Therapy (NAT) Prevents Metastases Across Conditions

Neutrophil-activating therapy (NAT) was shown by Linde et al., 2023 to reduce distant metastases in melanoma and mammary carcinoma models, but the mechanism of action and timing/dosing requirements for treatment remained unclear. To better understand these mechanisms, an experimental metastasis model was used in which B16F0 melanoma cells expressing the fluorescent protein tdTomato are first implanted subcutaneously in the left flank of B6 mice, followed by intravenous implantation of B16F0 tdTomato cells into the tail vein. These intravenously injected cells travel through vasculature and extravasate at the first capillary bed they arrive at, in the lungs. About 10 days after seeding, lungs are harvested and imaged through fluorescent microscopy, and metastases counted using ImageJ software. To treat these B16F0 tdTomato melanoma-bearing mice, NAT was used, which consisted of 1 μg recombinant mouse TNF (BioLegend, amino acids Leu80-Leu235), 100 μg anti-mouse CD40 agonist antibody (clone FGK4.5/FGK45, Bio X Cell), and 100 μg anti-mouse/human gp75 TYRP1/TRP1 (clone TA99, Bio X Cell) (melanoma binding antibody).

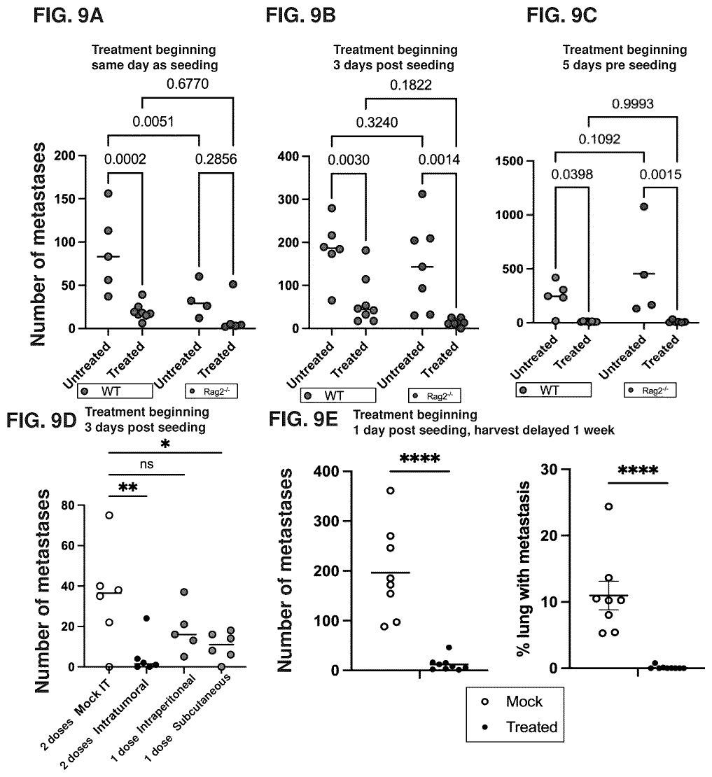

It was first investigated whether adaptive immune cells were required for the reduction of metastases and found that when NAT was administered with the same dosage and timing as described by Linde et al., 2023 (FIG. 9A), NAT reduced metastases in both B6 and Rag2^{-/-} mice, indicating a T cell-independent response was occurring. To verify that these effects were not mere artifacts of the short time between seeding and treatment, treatment was then performed with NAT three and five days after metastatic seeding. NAT still significantly reduced metastatic growth in B6 and Rag2^{-/-} mice (FIG. 9B). Mice were next treated with NAT five and three days prior to metastatic seeding and found that treated B6 and Rag2^{-/-} mice could prevent future metastatic seeding (FIG. 9C). Because NAT was originally developed for the treatment of solid tumors, it was next determined whether the dosage number and treatment site could be altered. A single dose of NAT three days after metastatic seeding injected subcutaneously on contralateral flank relative to the primary tumor was sufficient to significantly reduce metastases (FIG. 9D).

Finally, to assess the durability of the protection from metastases, the timeline of metastatic growth was extended for another week, harvesting lungs 16 days after seeding. NAT treatment resulted in a profound reduction of metastases in this experiment, showing reduction of metastatic coverage of lungs from approximately 11% to 0% (FIG. 9E). Together, these experiments highlight the potential for NAT to prevent metastases for at least two weeks post treatment. The efficacy of subcutaneous administration, and the high efficacy in mice lacking adaptive immunity show the possibility for usage in patients with inaccessible tumors and in immunocompromised patients.

### REFERENCES

1. Coffelt, S. B., Wellenstein, M. D., and de Visser, K. E. (2016). Neutrophils in cancer: neutral no more. Nat. Rev. Cancer 16, 431-446. https://doi. org/10. 1038/nrc.2016.52.
2. Jaillon, S., Ponzetta, A., Di Mitri, D., Santoni, A., Bonecchi, R., and Mantovani, A. (2020). Neutrophil diversity and plasticity in tumour progression and therapy. Nat. Rev. Cancer 20, 485-503. https://doi.org/10.1038/s41568-020-0281-y.
3. Mantovani, A., Cassatella, M. A., Costantini, C., and Jaillon, S. (2011). Neutrophils in the activation and regulation of innate and adaptive immunity. Nat. Rev. Immunol. 11, 519-531. https://doi.org/10.1038/nri3024.
4. Gentles, A. J., Newman, A. M., Liu, C. L., Bratman, S. V., Feng, W., Kim, D., Nair, V. S., Xu, Y., Khuong, A., Hoang, C. D., et al. (2015). The prognostic landscape of genes and infiltrating immune cells across human cancers. Nat. Med. 21, 938-945. https://doi.org/10.1038/nm.3909.
5. Shaul, M. E., andFridlender, Z. G. (2019). Tumour-associatedneutrophilsin patients with cancer. Nat. Rev. Clin. Oncol. 16, 601-620. https://doi.org/10.1038/s41571-019-0222-4.
6. Pekarek, L. A., Starr, B. A., Toledano, A. Y., and Schreiber, H. (1995). Inhibition of tumor growth by elimination of granulocytes. J. Exp. Med. 181, 435-440. https://doi.org/10.1084/jem.181.1.435.
7. Jablonska, J., Leschner, S., Westphal, K., Lienenklaus, S., and Weiss, S. (2010). Neutrophils responsive to endogenous IFN-b regulate tumor angiogenesis and growth in a mouse tumor model. J. Clin. Invest. 120, 1151-1164. https://doi.org/10.1172/JCI37223.
8. Nozawa, H., Chiu, C., and Hanahan, D. (2006). Infiltrating neutrophils mediate the initial angiogenic switch in a mouse model of multistage carcinogenesis. Proc. Natl. Acad. Sci. USA 103, 12493-12498. https://doi.org/10.1073/pnas.0601807103.
9. Coffelt, S. B., Kersten, K., Doornebal, C. W., Weiden, J., Vrijland, K., Hau, C.-S., Verstegen, N. J. M., Ciampricotti, M., Hawinkels, L. J. A. C., Jonkers, J., and de Visser, K. E. (2015). IL-17-producing gd T cells and neutrophils conspire to promote breast cancer metastasis. Nature 522, 345-348. https://doi.org/10.1038/naturel4282.
10. Wculek, S. K., and Malanchi, I. (2015). Neutrophils support lung colonization of metastasis-initiating breast cancer cells. Nature 528, 413-417. https://doi.org/10.1038/nature16140.
11. Szczerba, B. M., Castro-Giner, F., Vetter, M., Krol, I., Gkountela, S., Landin, J., Scheidmann, M. C., Donato, C., Scherrer, R., Singer, J., et al. (2019). Neutrophils escort circulating tumour cells to enable cell cycle progression. Nature 566, 553-557. https://doi.org/10.1038/s41586-019-0915-y.
12. Schmielau, J., and Finn, O. J. (2001). Activated granulocytes and granulocyte-derived hydrogen peroxide are the underlying mechanism of suppression of t-cell function in advanced cancer patients. Cancer Res. 61, 4756-4760.
13. Mishalian, I., Bayuh, R., Eruslanov, E., Michaeli, J., Levy, L., Zolotarov, L., Singhal, S., Albelda, S. M., Granot, Z., and Fridlender, Z. G. (2014). Neutrophils recruit regulatory T-cells into tumors via secretion of CCL17—a new mechanism of impaired antitumor immunity. Int. J. Cancer 135, 1178-1186. https://doi.org/10.1002/ijc.28770.
14. Veglia, F., Sanseviero, E., and Gabrilovich, D. I. (2021). Myeloid-derived suppressor cells in the era of increasing myeloid cell diversity. Nat. Rev. Immunol. 21, 485-498. https://doi.org/10.1038/s41577-020-00490-y.
15. Kusmartsev, S., Nefedova, Y., Yoder, D., and Gabrilovich, D. I. (2004). Antigen-specific inhibition of CD8⁻ T cell response by immature myeloid cells in cancer is mediated by reactive oxygen species. J. Immunol. 172, 989-999. https://doi.org/10.4049/jimmunol.172.2.989.
16. Youn, J.-I., Nagaraj, S., Collazo, M., and Gabrilovich, D. I. (2008). Subsets of myeloid-derived suppressor cells in tumor-bearing mice. J. Immunol. 181, 5791-5802. https://doi.org/10.4049/jimmunol.181.8.5791.
17. Clark, R. A., and Klebanoff, S. J. (1975). Neutrophil-mediated tumor cell cytotoxicity: role of the peroxidase system. J. Exp. Med. 141, 1442-1447. https://doi.org/10.1084/jem.141.6.1442.
18. Blaisdell, A., Crequer, A., Columbus, D., Daikoku, T., Mittal, K., Dey, S. K., and Erlebacher, A. (2015). Neutrophils oppose uterine epithelial carcinogenesis via debridement of hypoxic tumor cells. Cancer Cell 28, 785-799. https://doi.org/10.1016/j.ccell.2015.11.005.
19. Eruslanov, E. B., Bhojnagarwala, P. S., Quatromoni, J. G., Stephen, T. L., Ranganathan, A., Deshpande, C., Akimova, T., Vachani, A., Litzky, L., Hancock, W. W., et al. (2014). Tumor-associated neutrophils stimulate T cell responses in early-stage human lung cancer. J. Clin. Invest. 124, 5466-5480. https://doi.org/10.1172/jci77053.
20. Singhal, S., Bhojnagarwala, P. S., O'Brien, S., Moon, E. K., Garfall, A. L., Rao, A. S., Quatromoni, J. G., Stephen, T. L., Litzky, L., Deshpande, C., et al. (2016). Origin and role of a subset of tumor-associated neutrophils with antigen-presenting cell features in early-stage human lung cancer. Cancer Cell 30, 120-135. https://doi.org/10.1016/j.ccell.2016.06.001.
21. Ponzetta, A., Carriero, R., Carnevale, S., Barbagallo, M., Molgora, M., Perucchini, C., Magrini, E., Gianni, F., Kunderfranco, P., Polentarutti, N., et al. (2019). Neutrophils driving unconventional T cells mediate resistance against murine sarcomas and selected human tumors. Cell 178, 346-360.e24. https://doi.org/10.1016/j.cell.2019.05.047.
22. Costanzo-Garvey, D. L., Keeley, T., Case, A. J., Watson,G. F., Alsamraae,M., Yu, Y., Su, K., Heim, C. E., Kielian, T., Morrissey, C., et al. (2020). Neutrophils are mediators of metastatic prostate cancer progression in bone. Cancer immunology, immunotherapy. Cancer Immunol. Immunother. 69, 1113-1130. https://doi.org/10.1007/s00262-020-02527-6.
23. Granot, Z., Henke, E., Comen, E. A., King, T. A., Norton, L., and Benezra, R. (2011). Tumor entrained neutrophils inhibit seeding in the premetastatic lung. Cancer Cell 20, 300-314. https://doi.org/10.1016/j.ccr.2011.08.012.
24. Fridlender, Z. G., Sun, J., Kim, S., Kapoor, V., Cheng, G., Ling, L., Worthen, G. S., and Albelda, S. M. (2009). Polarization of tumor-associated neutrophil phenotype by TGF-beta: “N1” versus “N2” TAN. Cancer Cell 16, 183-194. https://doi.org/10.1016/j.ccr.2009.06.017.
25. Veglia, F., Tyurin, V. A., Blasi, M., De Leo, A., Kossenkov, A. V., Donthireddy, L., To, T. K. J., Schug, Z., Basu, S., Wang, F., et al. (2019). Fatty acid transport protein 2 reprograms neutrophils in cancer. Nature 569, 73-78. https://doi.org/10.1038/s41586-019-1118-2.
26. Yang, J., Kumar, A., Vilgelm, A. E., Chen, S.-C., Ayers, G. D., Novitskiy, S. V., Joyce, S., and Richmond, A. (2018). Loss of CXCR4 in myeloid cells enhances antitumor immunity and reduces melanoma growth through N K cell and FASL mechanisms. Cancer Immunol. Res. 6, 1186-1198. https://doi.org/10.1158/2326-6066.Cir-18-0045.
27. Sagiv, J. Y., Michaeli, J., Assi, S., Mishalian, I., Kisos, H., Levy, L., Damti, P., Lumbroso, D., Polyansky, L., Sionov, R. V., et al. (2015). Phenotypic diversity and plasticity in circulating neutrophil subpopulations in cancer. Cell Rep. 10, 562-573. https://doi.org/10.1016/j.celrep.2014.12.039.
28. Steele, C. W., Karim, S. A., Leach, J. D. G., Bailey, P., Upstill-Goddard, R., Rishi, L., Foth, M., Bryson, S., McDaid, K., Wilson, Z., et al. (2016). CXCR2 inhibition profoundly suppresses metastases and augments immunotherapy in pancreatic ductal adenocarcinoma. Cancer Cell 29, 832-845. https://doi.org/10.1016/j.ccell.2016.04.014.
29. Acharyya, S., Oskarsson, T., Vanharanta, S., Malladi, S., Kim, J., Morris, P. G., Manova-Todorova, K., Leversha, M., Hogg, N., Seshan, V. E., et al. (2012). A CXCL1 paracrine network links cancer chemoresistance and metastasis. Cell 150, 165-178. https://doi.org/10.1016/j.cell.2012.04.042.
30. Pylaeva, E., Harati, M. D., Spyra, I., Bordbari, S., Strachan, S., Thakur, B. K., Höing, B., Franklin, C., Skokowa, J., Welte, K., et al. (2019). NAMPT signaling is critical for the proangiogenic activity of tumor-associated neutrophils. Int. J. Cancer 144, 136-149. https://doi.org/10.1002/ijc.31808.
31. Shrestha, S., Noh, J. M., Kim, S.-Y., Ham, H.-Y., Kim, Y.-J., Yun, Y.-J., Kim, M.-J., Kwon, M.-S., Song, D.-K., and Hong, C.-W. (2016). Angiotensin converting enzyme inhibitors and angiotensin I I receptor antagonist attenuate tumor growth via polarization of neutrophils toward an antitumor phenotype. Oncoimmunology 5, e1067744. https://doi.org/10.1080/2162402X.2015.1067744.
32. Matlung, H. L., Babes, L., Zhao, X. W., van Houdt, M., Treffers, L. W., van Rees, D. J., Franke, K., Schornagel, K., Verkuijlen, P., Janssen, H., et al. (2018). Neutrophils kill antibody-opsonized cancer cells by trogoptosis. Cell Rep. 23, 3946-3959.e6. https://doi.org/10.1016/j.celrep.2018.05.082.
33. Albanesi, M., Mancardi, D. A., Jo*usson, F., Iannascoli, B., Fiette, L., Di Santo, J. P., Lowell, C. A., and Bruhns, P. (2013). Neutrophils mediate antibody-induced antitumor effects in mice. Blood 122, 3160-3164. https://doi.org/10.1182/blood-2013-04-497446.
34. Miralda, I., Uriarte, S. M., and McLeish, K. R. (2017). Multiple phenotypic changes define neutrophil priming. Front. Cell. Infect. Microbiol. 7, 217. https://doi.org/10.3389/fcimb.2017.00217.
35. Woodfin, A., Beyrau, M., Voisin, M.-B., Ma, B., Whiteford, J. R., Hordijk, P. L., Hogg, N., and Nourshargh, S. (2016). ICAM-1-expressing neutrophils exhibit enhanced effector functions in murine models of endotoxemia. Blood 127, 898-907. https://doi.org/10.1182/blood-2015-08-664995.
36. Condliffe, A. M., Chilvers, E. R., Haslett, C., and Dransfield, I. (1996). Priming differentially regulates neutrophil adhesion molecule expression/function. Immunology 89, 105-111. https://doi.org/10.1046/j.1365-2567. 1996.d01-711.x.
37. Khan, S. Y., Kelher, M. R., Heal, J. M., Blumberg, N., Boshkov, L. K., Phipps, R., Gettings, K. F., McLaughlin, N. J., and Silliman, C. C. (2006). Soluble CD40 ligand accumulates in stored blood components, primes neutrophils through CD40, and is a potential cofactor in the development of transfusion-related acute lung injury. Blood 108, 2455-2462. https://doi.org/10. 1182/blood-2006-04-017251.
38. Evrard, M., Kwok, I. W. H., Chong, S. Z., Teng, K. W. W., Becht, E., Chen, J., Sieow, J. L., Penny, H. L., Ching, G. C., Devi, S., et al. (2018). Developmental analysis of bone marrow neutrophils reveals populations specialized in expansion, trafficking, and effector functions. Immunity 48, 364-379.e8. https://doi.org/10.1016/j.immuni.2018.02.002.
39. Kwok, I., Becht, E., Xia, Y., Ng, M., Teh, Y. C., Tan, L., Evrard, M., Li, J. L. Y., Tran, H. T. N., Tan, Y., et al. (2020). Combinatorial single-cell analyses of granulocyte-monocyte progenitor heterogeneity reveals an early unipotent neutrophil progenitor. Immunity 53, 303-318.e5. https://doi.org/10.1016/j.immuni.2020.06.005.
40. Challen, G. A., Boles, N., Lin, K. K. Y., and Goodell, M. A. (2009). Mouse hematopoietic stem cell identification and analysis. Cytometry A. 75, 14-24. https://doi.org/10.1002/cyto.a.20674.
41. Zhou, G., Peng, K., Song, Y., Yang, W., Shu, W., Yu, T., Yu, L., Lin, M., Wei, Q., Chen, C., et al. (2018). CD177+ neutrophils suppress epithelial cell tumourigenesis in colitis-associated cancer and predict good prognosis in colorectal cancer. Carcinogenesis 39, 272-282. https://doi.org/10.1093/carcin/bgxl42.
42. Engblom, C., Pfirschke, C., Zilionis, R., Da Silva Martins, J., Bos, S. A., Courties, G., Rickelt, S., Severe, N., Baryawno, N., Faget, J., et al. (2017). Osteoblasts remotely supply lung tumors with cancer-promoting SiglecF(high) neutrophils. Science 358, eaa15081. https://doi.org/10. 1126/science.aa15081.
43. Veglia, F., Hashimoto, A., Dweep, H., Sanseviero, E., De Leo, A., Tcyganov, E., Kossenkov, A., Mulligan, C., Nam, B., Masters, G., et al. (2021). Analysis of classical neutrophils and polymorphonuclear myeloid-derived suppressor cells in cancer patients and tumor-bearing mice. J. Exp. Med. 218, e20201803. https://doi.org/10.1084/jem.20201803.
44. Yajuk, O., Baron, M., Toker, S., Zelter, T., Fainsod-Levi, T., and Granot, Z. (2021). The P D-L1/PD-1 Axis blocks neutrophil cytotoxicity in cancer. Cells 10, 1510.
45. Gavillet, M., Martinod, K., Renella, R., Harris, C., Shapiro, N. I., Wagner, D. D., and Williams, D. A. (2015). Flow cytometric assay for direct quantification of neutrophil extracellular traps in blood samples. Am. J. Hematol. 90, 1155-1158. https://doi.org/10.1002/ajh.24185.
46. Herndler-Brandstetter, D., Ishigame, H., Shinnakasu, R., Plajer, V., Stecher, C., Zhao, J., Lietzenmayer, M., Kroehling, L., Takumi, A., Kometani, K., et al. (2018). KLRG1+ effector CD8+ T cells lose KLRG1, differentiate into all memory T cell lineages, and convey enhanced protective immunity. Immunity 48, 716-729.e8. https://doi.org/i0.1016/j.immuni.2018.03.015.
47. Dunkelberger, J. R., and Song, W. C. (2010). Complement and its role in innate and adaptive immune responses. Cell Res. 20, 34-50. https://doi. org/10.1038/cr.2009.139.
48. Brandt, S. L., and Serezani, C. H. (2017). Too much of a good thing: how modulating LTB(4) actions restore host defense in homeostasis or disease. Semin. Immunol. 33, 37-43. https://doi.org/10.1016/j.smim.2017.08.006.
49. Roumenina, L. T., Daugan, M. V., Petitprez, F., Saute's-Fridman, C., and Fridman, W. H. (2019). Context-dependent roles of complement in cancer. Nat. Rev. Cancer 19, 698-715. https://doi.org/10.1038/s41568-019-0210-0.
50. Battelli, M. G., Polito, L., Bortolotti, M., and Bolognesi, A. (2016). Xanthine oxidoreductase in cancer: more than a differentiation marker. Cancer Med. 5, 546-557. https://doi.org/10.1002/cam4.601.
51. Wang, Y., Wu, J., Newton, R., Bahaie, N. S., Long, C., and Walcheck, B. (2013). ADAM17 cleaves CD16b (FcgRIIIb) in human neutrophils. Biochim. Biophys. Acta 1833, 680-685. https://doi.org/10.1016/j.bbamcr.2012. 11.027.
52. Ajona, D., Ortiz-Espinosa, S., Moreno, H., Lozano, T., Pajares, M. J., Agorreta, J., Bertolo, C., Lasarte, J. J., Vicent, S., Hoehlig, K., et al. (2017). A combined PD-1/C5a blockade synergistically protects against lung cancer growth and metastasis. Cancer Discov. 7, 694-703. https://doi.org/10.1158/2159-8290.Cd-16-1184.
53. Corrales, L., Ajona, D., Rafail, S., Lasarte, J. J., Riezu-Boj, J. I., Lambris, J. D., Rouzaut, A., Pajares, M. J., Montuenga, L. M., and Pio, R. (2012). Anaphylatoxin C5a creates a favorable microenvironment for lung cancer progression. J. Immunol. 189, 4674-4683. https://doi.org/10.4049/jimmunol.1201654.
54. Vadrevu, S. K., Chintala, N. K., Sharma, S. K., Sharma, P., Cleveland, C., Riediger, L., Manne, S., Fairlie, D. P., Gorczyca, W., Almanza, O., et al. (2014). Complement c5a receptor facilitates cancer metastasis by altering T-cell responses in the metastatic niche. Cancer Res. 74, 3454-3465. https://doi.org/10.1158/0008-5472.Can-14-0157.
55. Piao, C., Cai, L., Qiu, S., Jia, L., Song, W., and Du, J. (2015). Complement 5a enhances hepatic metastases of colon cancer via monocyte chemoattractant protein-i-mediated inflammatory cell infiltration. J. Biol. Chem. 290, 10667-10676. https://doi.org/10.1074/jbc.M114.612622.
56. Markiewski, M. M., DeAngelis, R. A., Benencia, F., Ricklin-Lichtsteiner, S. K., Koutoulaki, A., Gerard, C., Coukos, G., and Lambris, J. D. (2008). Modulation of the antitumor immune response by complement. Nat. Immunol. 9, 1225-1235. https://doi.org/10.1038/ni.1655.
57. Satpathy, S. R., Jala, V. R., Bodduluri, S. R., Krishnan, E., Hegde, B., Hoyle, G. W., Fraig, M., Luster, A. D., and Haribabu, B. (2015). Crystalline silicainduced leukotriene B4-dependent inflammation promotes lung tumour growth. Nat. Commun. 6, 7064. https://doi.org/10.1038/ncomms8064.
58. Yokota, Y., Inoue, H., Matsumura, Y., Nabeta, H., Narusawa, M., Watanabe, A., Sakamoto, C., Hijikata, Y., Iga-Murahashi, M., Takayama, K., et al. (2012). Absence of LTB₄/BLT1 axis facilitates generation of mouse G M-CSF-induced long-lasting antitumor immunologic memory by enhancing innate and adaptive immune systems. Blood 120, 3444-3454. https://doi.org/10.1182/blood-2011-10-383240.
59. Tian, W., Jiang, X., Kim, D., Guan, T., Nicolls, M. R., and Rockson, S. G. (2020). Leukotrienes in tumor-associated inflammation. Front. Pharmacol. 11, 1289. https://doi.org/10.3389/fphar.2020.01289.
60. Akk, A., Springer, L. E., Yang, L., Hamilton-Burdess, S., Lambris, J. D., Yan, H., Hu, Y., Wu, X., Hourcade, D. E., Miller, M. J., and Pham, C. T. N. (2019). Complement activation on neutrophils initiates endothelial adhesion and extravasation. Mol. Immunol. 114, 629-642. https://doi.org/10.1016/j.molimm.2019.09.011.
61. Camous, L., Roumenina, L., Bigot, S., Brachemi, S., Fre meaux-Bacchi, V., Lesavre, P., and Halbwachs-Mecarelli, L. (2011). Complement alternative pathway acts as a positive feedback amplification of neutrophil activation. Blood 117, 1340-1349. https://doi.org/10.1182/blood-2010-05-283564.
62. La€mmermann, T., Afonso, P. V., Angermann, B. R., Wang, J. M., Kastenmu€ller, W., Parent, C. A., and Germain, R. N. (2013). Neutrophil swarms require LTB₄ and integrins at sites of cell death in vivo. Nature 498, 371-375. https://doi.org/10.1038/naturel2175.
63. Lee, E. K. S., Gillrie, M. R., Li, L., Arnason, J. W., Kim, J. H., Babes, L., Lou, Y., Sanati-Nezhad, A., Kyei, S. K., Kelly, M. M., et al. (2018). Leukotriene B4-mediated neutrophil recruitment causes pulmonary capillaritis during lethal fungal sepsis. Cell Host Microbe 23, 121-133.e4. https://doi.org/10.1016/j.chom.2017.11.009.
64. Uderhardt, S., Martins, A. J., Tsang, J. S., La€mmermann, T., and Germain, R. N. (2019). Resident macrophages cloak tissue microlesions to prevent neutrophil-driven inflammatory damage. Cell 177, 541-555.e17. https://doi.org/10.1016/j.cell.2019.02.028.
65. Sadik, C. D., Kim, N. D., Iwakura, Y., and Luster, A. D. (2012). Neutrophils orchestrate their own recruitment in murine arthritis through C5aR and FcgR signaling. Proc. Natl. Acad. Sci. USA 109, E3177-E3185. https://doi.org/10.1073/pnas.1213797109.
66. Kim, N. D., Chou, R. C., Seung, E., Tager, A. M., and Luster, A. D. (2006). A unique requirement for the leukotriene B4 receptor BLT1 for neutrophil recruitment in inflammatory arthritis. J. Exp. Med. 203, 829-835. https://doi.org/10.1084/jem.20052349.
67. Kimura, Y., Zhou, L., Miwa, T., and Song, W.-C. (2010). Genetic and therapeutic targeting of properdin in mice prevents complement-mediated tissue injury. J. Clin. Invest. 120, 3545-3554. https://doi.org/10.1172/JCI41782.
68. Kubo, H., Morgenstern, D., Quinian, W. M., Ward, P. A., Dinauer, M. C., and Doerschuk, C. M. (1996). Preservation of complement-induced lung injury in mice with deficiency of NADPH oxidase. J. Clin. Invest. 97, 2680-2684. https://doi.org/10.1172/jci118718.
69. Carmi, Y., Spitzer, M. H., Linde, I. L., Burt, B. M., Prestwood, T. R., Perlman, N., Davidson, M. G., Kenkel, J. A., Segal, E., Pusapati, G. V., et al. (2015). Allogeneic IgG combined with dendritic cell stimuli induce antitumour T-cell immunity. Nature 521, 99-104. https://doi.org/10.1038/nature14424.
70. Spitzer, M. H., Carmi, Y., Reticker-Flynn, N. E., Kwek, S. S., Madhireddy, D., Martins, M. M., Gherardini, P. F., Prestwood, T. R., Chabon, J., Bendall, S. C., et al. (2017). Systemic immunity is required for effective cancer immunotherapy. Cell 168, 487-502.e15. https://doi.org/10.1016/j.cell.2016.12.022.
71. Bonaventura, P., Shekarian, T., Alcazer, V., Valladeau-Guilemond, J., Valsesia-Wittmann, S., Amigorena, S., Caux, C., and Depil, S. (2019). Cold tumors: a therapeutic challenge for immunotherapy. Front. Immunol. 10, 168. https://doi.org/10.3389/fimmu.2019.00168.
72. Arroyo-Crespo, J. J., Armiñán, A., Charbonnier, D., Deladriere, C., PalominoScha€tzlein, M., Lamas-Domingo, R., Forteza, J., Pineda-Lucena, A., and Vicent, M. J. (2019). Characterization of triple-negative breast cancer preclinical models provides functional evidence of metastatic progression. Int. J. Cancer 145, 2267-2281. https://doi.org/10.1002/ijc.32270.
73. Roberts, N. J., Zhou, S., Diaz, L. A., Jr., and Holdhoff, M. (2011). Systemic use of tumor necrosis factor alpha as an anticancer agent. Oncotarget 2, 739-751. https://doi.org/10.18632/oncotarget.344.
74. Miwa, T., Sato, S., Gullipalli, D., Nangaku, M., and Song, W.-C. (2013). Blocking properdin, the alternative pathway, and anaphylatoxin receptors ameliorates renal ischemia-reperfusion injury in decay-accelerating factor and CD59 double-knockout mice. J. Immunol. 190, 3552-3559. https://doi.org/10.4049/jimmunol.1202275.
75. Ueda, Y., Miwa, T., Ito, D., Kim, H., Sato, S., Gullipalli, D., Zhou, L., Golla, M., Song, D., Dunaief, J. L., et al. (2019). Differential contribution of C5aR and C5b-9 pathways to renal thrombic microangiopathy and macrovascular thrombosis in mice carrying an atypical hemolytic syndrome-related factor H mutation. Kidney Int. 96, 67-79. https://doi.org/10.1016/j.kint.2019. 01.009.
76. Ran, F. A., Hsu, P. D., Wright, J., Agarwala, V., Scott, D. A., and Zhang, F. (2013). Genome engineering using the CRISPR-Cas9 system. Nat. Protoc. 8, 2281-2308. https://doi.org/10.1038/nprot.2013.143.
77. Sato, T., Vries, R. G., Snippert, H. J., van de Wetering, M., Barker, N., Stange, D. E., van Es, J. H., Abo, A., Kujala, P., Peters, P. J., and Clevers, H. (2009). Single Lgr5 stem cells build crypt-villus structures in vitro without a mesenchymal niche. Nature 459, 262-265. https://doi.org/10. 1038/nature07935.
78. Sjögren, J., Lood, R., and Na€geli, A. (2020). Onenzymaticremodelingof IgG glycosylation; unique tools with broad applications. Glycobiology 30, 254-267. https://doi.org/10.1093/glycob/cwz085.
79. Van den Berg, C. W., Aerts, P. C., and Van Dijk, H. (1991). In vivo anti-complementary activities of the cobra venom factors from Naja naja and Naja haje. J. Immunol. Methods 136, 287-294. https://doi.org/10.1016/00221759(91)90015-8.
80. Paschall, A. V., and Liu, K. (2016). An orthotopic mouse model of spontaneous breast cancer metastasis. J. Vis. Exp. https://doi.org/10. 3791/54040.
81. Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize offtarget effects of CRISPR-Cas9. Nat. Biotechnol. 34, 184-191. https://doi.org/10.1038/nbt.3437.

What is claimed is:

1. A composition comprising: a TNF agent; a CD40 agent; and a tumor-binding antibody.

2. A kit comprising: a TNF agent; a CD40 agent; and a tumor-binding antibody.

3. The composition of claim 1 or the kit of claim 2, wherein the TNF agent is TNF.

4. The composition of claim 1 or the kit of claim 2, wherein the CD40 agent is a CD40 agonist antibody.

5. The composition or kit of claim 4, wherein the CD40 agent is selected from the group consisting of selicrelmab, APX005M, ChiLob 7/4, ADC-1013, Sea-CD40, and CDX1140.

6. The composition of claim 1 or the kit of claim 2, wherein the tumor-binding antibody is selected from the group consisting of Amivantamab, Atezolizumab, Avelumab, Belantamab mafodotin, Bevacizumab, Blinatumomab, Brentuximab vedotin, Capromab, CatumaxomabW, Cemiplimab, Cetuximab, Daratumumab, Dinutuximab, Dostarlimab, Durvalumab, Elotuzumab, Ertumaxomab, Etaracizumab, Glofitamab, Inebilizumab, Inotuzumab ozogamicin, Ipilimumab, Isatuximab, Margetuximab, Mogamulizumab, Moxetumomab pasudotox, Necitumumab, Nimotuzumab, Nivolumab, Olaratumab, Panitumumab, Pembrolizumab, Pertuzumab, Polatuzumab vedotin, Racotumomab, Ramucirumab, Retifanlimab, Sacituzumab govitecan, Siltuximab, Talquetamab, Teclistamab, Trastuzumab, Trastuzumab duocarmazine, Trastuzumab emtansine, and Tremelimumab.

7. A method of treating cancer in a subject in need of treatment therefor, comprising administering to the subject: a TNF agent, a CD40 agent, and a tumor-binding antibody.

8. The method of claim 7, wherein the cancer is selected from the group consisting of lung, prostate, breast, bladder, colon, ovarian, pancreas, kidney, liver, glioblastoma, medulloblastoma, leiomyosarcoma, head & neck squamous cell carcinomas, melanomas, carcinomas; soft tissue tumors; sarcomas; teratomas; melanomas; leukemias; lymphomas; and brain cancers, including minimal residual disease, and including both primary and metastatic tumors; cancers of the breast, pancreas, lung, prostate, and colon comprising adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; Carcinomas of the prostrate, pancreas, colon, brain lung, breast, skin, and metastases of the foregoing.

9. The method of claim 7, wherein the TNF agent is TNF.

10. The method of claim 7, wherein the CD40 agent is a CD40 agonist antibody.

11. The method of claim 10, wherein the CD40 agnostic antibody is selected from the group consisting of selicrelmab, APX005M, ChiLob 7/4, ADC-1013, Sea-CD40, and CDX1140.

12. The method of claim 7, wherein the tumor-binding antibody is selected from the group consisting of Amivantamab, Atezolizumab, Avelumab, Belantamab mafodotin, Bevacizumab, Blinatumomab, Brentuximab vedotin, Capromab, CatumaxomabW, Cemiplimab, Cetuximab, Daratumumab, Dinutuximab, Dostarlimab, Durvalumab, Elotuzumab, Ertumaxomab, Etaracizumab, Glofitamab, Inebilizumab, Inotuzumab ozogamicin, Ipilimumab, Isatuximab, Margetuximab, Mogamulizumab, Moxetumomab pasudotox, Necitumumab, Nimotuzumab, Nivolumab, Olaratumab, Panitumumab, Pembrolizumab, Pertuzumab, Polatuzumab vedotin, Racotumomab, Ramucirumab, Retifanlimab, Sacituzumab govitecan, Siltuximab, Talquetamab, Teclistamab, Trastuzumab, Trastuzumab duocarmazine, Trastuzumab emtansine, and Tremelimumab.

13. The method of claim 7, wherein the TNF agent; the CD40 agent; and the tumor-binding antibody are co-administered substantially simultaneously.

14. The method of claim 7, wherein the TNF agent; the CD40 agent; and the tumor-binding antibody are administered intratumorally or peritumorally.

15. The method of any one of claims 7-13, wherein said administering comprises systemic administration.

16. The method of any one of claims 7-13, wherein said administering comprises subcutaneous administration.
